(19) United States
(12) Patent Application Publication
(10) Pub. No.: US 2006/0252766 A1
(43) Pub. Date: Nov. 9, 2006

(54) Methods using a combination of 3-heteroaryl-2-indolinone and a cyclooxygenase-2 inhibitor for the treatment of neoplasia

(75) Inventors: Jaime L. Masferrer, Ballwin, MO (US); Julie M. Cherrington, La Jolla, CA (US); Kathleen M. Leahy, St. Louis, MO (US); Ben S. Zweifel, Eureka, MO (US)

Correspondence Address: HARNESS, DICKEY, & PIERCE, P.L.C, 7700 BONHOMME, STE 400, ST. LOUIS, MO, 63105, US

(73) Assignee: PFIZER INC

(21) Appl. No.: 10/514,745

(22) PCT Filed: May 15, 2003
(86) PCT No.: PCT/US03/15582

§ 371 Date: Mar. 14, 2006

(30) Foreign Application Priority Data
May 16, 2002 (US) 10/150546

(51) Int. Cl. A61K 31/501; A61K 31/4709; A61K 31/4196; A61K 31/4178; A61K 31/416; A61K 31/415; A61K 31/41; A61K 31/405; A61K 31/382

(52) U.S. Cl. 514/252.05; 514/312; 514/406; 514/414; 514/364; 514/383; 514/397; 514/381; 514/362; 514/456; 514/432

(57) ABSTRACT

The present invention provides methods and compositions useful for treatment or prevention of neoplasia by administering a combination comprising a 3-heteroaryl-2-indolinone compound and a COX-2 selective inhibitor. Further provided are compositions, pharmaceutical compositions, and kits for treatment and prevention of neoplasia.

### FIELD OF THE INVENTION

The present invention relates to compositions and methods employing combinations of a 3-heteroaryl-2-indolinone compound and a cyclooxygenase-2 (COX-2) selective inhibitor for treatment of neoplasia.

### BACKGROUND OF THE INVENTION

A neoplasm, or tumor, is an abnormal, unregulated, and disorganized proliferation of cell growth. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now the second leading cause of death in the United States where over 8,000,000 individuals have been diagnosed with some form of cancer. In 1995, cancer accounted for 23.3% of all deaths in the United States. (See U.S. Dept. of Health and Human Services, National Center for Health Statistics, Health United States 1996-97 and Injury Chartbook 117 (1997)).

Cancer is not fully understood on the molecular level. It is known that exposure of a cell to a carcinogen such as certain viruses, chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called protooncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

Cancer is now primarily treated with one or a combination of three types of therapies: surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia.

Chemotherapy involves the disruption of cell replication or cell metabolism. It is used most often in the treatment of breast, lung, and testicular cancer. The adverse effects of systemic chemotherapy used in the treatment of neoplastic disease are most feared by patients undergoing treatment for cancer. Of these adverse effects nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications (see M. D. Abeloff, et al: Alopecia and Cutaneous Complications. P. 755-56. In Abeloff, M. D., Armitage, J. O., Lichter, A. S., and Niederhuber, J. E. (eds) Clinical Oncology. Churchill Livingston, New York, 1992, for cutaneous reactions to chemotherapy agents), such as pruritis, urticaria, and angioedema; neurological complications; pulmonary and cardiac complications in patients receiving radiation or chemotherapy; and reproductive and endocrine complications.

Chemotherapy-induced side effects significantly impact the quality of life of the patient and may dramatically influence patient compliance with treatment.

Additionally, adverse side effects associated with chemotherapeutic agents are generally the major dose-limiting toxicity (DLT) in the administration of these drugs. For example, mucositis, is a major dose limiting toxicity for several anticancer agents, including the antimetabolite cytotoxic agents 5-FU, methotrexate, and antitumor antibiotics, such as doxorubicin. Many of these chemotherapy-induced side effects are severe, may lead to hospitalization, or require treatment with analgesics for the treatment of pain.

The adverse side effects induced by chemotherapeutic agents and radiation therapy have become of major importance to the clinical management of cancer patients.

U.S. Pat. No. 5,843,925 describes a method for inhibiting angiogenesis and endothelial cell proliferation using a 7-[substituted amino]-9-[(substituted glycyl)amido]-6-demethyl-6-deoxytetracycline.

U.S. Pat. No. 5,854,205 describes an isolated endostatin protein that is an inhibitor of endothelial cell proliferation and angiogenesis.

U.S. Pat. No. 5,863,538 describes methods and compositions for targeting tumor vasculature of solid tumors using immunological and growth factor-based reagents in combination with chemotherapy and radiation.

U.S. Pat. No. 5,837,682 describes the use of fragments of an endothelial cell proliferation inhibitor, angiostatin.

U.S. Pat. No. 5,861,372 describes the use of an aggregate endothelial inhibitor, angiostatin, and its use in inhibiting angiogenesis.

PCT/US97/09610 describes administration of an anti-endogin monoclonal antibody, or fragments thereof, which is conjugated to at least one angiogenesis inhibitor or antitumor agent for use in treating tumor and angiogenesis-associated diseases.

PCT/IL96/00012 describes a fragment of the Thrombin B-chain for the treatment of cancer.

PCT/US95/16855 describes compositions and methods of killing selected tumor cells using recombinant viral vectors.

Ravaud, A. et al. describes the efficacy and tolerance of interleukin-2 (IL-2), interferon alpha-2a, and fluorouracil in patients with metastatic renal cell carcinoma. .J. Clin. Oncol. 16, No. 8, 2728-32, 1998.

Stadler, W. M. et al. describes the response rate and toxicity of oral 13-cis-retinoic acid added to an outpatient regimen of subcutaneous interleukin-2 and interferon alpha in patients with metastatic renal cell carcinoma. J. Clin. Oncol. 16, No. 5, 1820-25, 1998

Rosenbeg, S. A. et al. describes treatment of patients with metastatic melanoma using chemotherapy with cisplatin, dacarbazine, and tamoxifen alone or in combination with interleukin-2 and interferon alpha-2b. J. Clin. Oncol. 17, No. 3, 968-75, 1999.

Tourani, J-M. et al describes treatment of renal cell carcinoma using interleukin-2, and interferon alpha-2a administered in combination with fluorouracil. J. Clin. Oncol. 16, No. 7, 2505-13, 1998.

Majewski, S. describes the anticancer action of retinoids, vitamin D3 and cytokines (interferons and interleukin-12) as related to the antiangiogenic and antiproliferative effects. J. Invest. Dermatol. 108, No. 4, 571, 1997.

Ryan, C. W. describes treatment of patients with metastatic renal cell cancer with GM-CSF, Interleukin-2, and interferon-alpha plus oral cis-retinoic acid in patients with metastatic renal cell cancer. J. Invest. Med. 46, No. 7, 274A, 1998.

Tai-Ping, D. describes potential anti-angiogenic therapies. Trends Pharmacol. Sci. 16, No. 2, 57-66,1995.

Brembeck, F. H. describes the use of 13-cis retinoic acid and interferon alpha to treat UICC stage III/IV pancreatic cancer. Gastroenterology 114, No. 4, Pt. 2, A569, 1998.

Brembeck, F. H. describes the use of 13-cis retinoic acid and interferon alpha in patients with advanced pancreatic carcinoma. Cancer 83, No.11, 2317-23,1998.

Mackean, M. J. describes the use of roquinimex (Linomide) and alpha interferon in patients with advanced malignant melanoma or renal carcinoma. Br. J. Cancer 78, No.12, 1620-23, 1998

Jayson, G. C. describes the use of interleukin 2 and interleukin-interferon alpha in advanced renal cancer. Br. J. Cancer 78, No. 3, 366-69, 1998.

Abraham, J. M. describes the use of Interleukin-2, interferon alpha and 5-fluorouracil in patients with metastatic renal carcinoma. Br. J. Cancer 78, Suppl. 2, 8, 1998.

Soori, G. S. describes the use of chemo-biotherapy with chlorambucil and alpha interferon in patients with non-hodgkins lymphoma. Blood 92, No.10, Pt. 2 Suppl. 1, 240b, 1998.

Enschede, S. H. describes the use of interferon, alpha added to an anthracycline-based regimen in treating low grade and intermediate grade non-hodgkin's lymphoma. Blood 92, No. 10, Pt. 1 Suppl. 1, 412a, 1998.

Schachter, J. describes the use of a sequential multi-drug chemotherapy and biotherapy with interferon alpha, a four drug chemotherapy regimen and GM-CSF. Cancer Biother. Radiopharm. 13, No. 3,155-64,1998.

Mross, K. describes the use of retinoic acid, interferon alpha and tamoxifen in metastatic breast cancer patients. J. Cancer Res. Clin. Oncology. 124 Suppl. 1 R123, 1998.

Muller, H. describes the use of suramin and tamoxifen in the treatment of advanced and metastatic pancreatic carcinoma. Eur. J. Cancer 33, Suppl. 8, S50, 1997.

Rodriguez, M. R. describes the use of taxol and cisplatin, and taxotere and vinorelbine in the treatment of metastatic breast cancer. Eur. J. Cancer 34, Suppl. 4, S17-S18, 1998.

Formenti, C. describes concurrent paclitaxel and radiation therapy in locally advanced breast cancer patients. Eur. J. Cancer 34, Suppl. 5, S39, 1998.

Durando, A. describes combination chemotherapy with paclitaxel (T) and epirubicin (E) for metastatic breast cancer. Eur. J. Cancer 34, Suppl. 5, S41, 1998.

Osaki, A. describes the use of a combination therapy with mitomycin-C, etoposide, doxifluridine and medroxyprogesterone acetate as second-line therapy for advanced breast cancer. Eur. J. Cancer 34, Suppl. 5, S59, 1998.

Lode, H. et al. describes Synergy between an antiangiogenic integrin alpha v antagonist and an antibody-cytokine fusion protein eradicates spontaneous tumor metastasis. Proc. Nat. Acad. Sci. USA., 96 (4), 1591-1596, 1999.

Giannis, A. et al describes Integrin antagonists and other low molecular weight compounds as inhibitors of angiogenesis: new drugs in cancer therapy. Angew. Chem. Int. Ed. Engl. 36(6), 588-590, 1997.

Takada, Y. et al describes the structures and functions of integrins. Jikken Igaku 14 (17), 2317-2322, 1996.

Varner, J. et al. Tumor angiogenesis and the role of vascular cell integrin alphavbeta3. Impt. Adv. Onc., 69-87 Ref:259. 1996.

The use of TNP-470 and minocycline in combination with cyclophasphamide, CDDP, or thiotepa have been observed to substantially increase the tumor growth delay in one pre-clinical solid tumor, model. (Teicher, B. A. et al., Breast Cancer Research and Treatment, 36: 227-236, 1995). Additionally, improved results were observed when the antiangiogenesis agents were used in combination with cyclophosphamide and fractionated radiation therapy. (Teicher, B. A. et al., European Journal of Cancer 32A(14): 2461-2466, 1996).

Neri et al. examined the use of AG-3340 in combination with carboplatin and taxol for the treatment of cancer. (Neri et al., Proc Am Assoc Can Res, Vol 39, 89 meeting, 302 1998).

U.S. Pat. No. 5,837,696 describes the use of tetracycline compounds to inhibit cancer growth.

WO 97/48,685 describes various substituted compounds that inhibit metalloproteases.

EP 48/9,577 describes peptidyl derivatives used to prevent tumor cell metastasis and invasion.

WO 98/25,949 describes the use of N5-substituted 5-amino-1,3,4-thiadiazole-2-thiols to inhibit metallopreteinase enzymes.

WO 99/21,583 describes a method of inhibiting metastases in patients having cancer in which wildtype p53 is predominantly expressed using a combination of radiation therapy and a selective matrix metalloproteinase-2 inhibitor.

WO 98/33,768 describes arylsulfonylamino hydroxamic acid derivatives in the treatment of cancer.

WO 98/30,566 describes cyclic sulfone derivatives useful in the treatment of cancer.

WO 98/34,981 describes arylsulfonyl hydroxamic acid derivatives useful in the treatment of cancer.

WO 98/33,788 discloses the use of carboxylic or hyroxamic acid derivatives for treatment of tumors.

WO 97/41,844 describes a method of using combinations of angiostatic compounds for the prevention and/or treatment of neovascularization in human patients.

EP 48/9,579 describes peptidyl derivatives with selective gelatinase action that may be of use in the treatment of cancer and to control tumor metastases.

WO 48/03,516 describes phasphinate based compounds useful in the treatment of cancer.

WO 93/24,475 describes sulphamide derivatives may be useful in the treatment of cancer to control the development of metastases.

WO 98/16,227 describes a method of using [Pyrozol-1-yl]benzenesulfonamides in the treatment of and prevention of neoplasia.

WO 98/22,101 describes a method of using [Pyrozol-1-yl]benzenesulfonamides as anti-angiogenic agents.

WO 96/03,385 describes 3,4,-Di substituted pyrazole compounds given alone or in combination with NSAIDs, steroids, 5-LO inhibitors, LTB4 antagonists, or LTA4 hydrolase inhibitors that may be useful in the treatment of cancer.

WO 98/47,890 describes substituted benzopyran derivatives that may be used alone or in combination with other active principles.

Compounds that selectively inhibit the cyclooxygenase-2 enzyme have been discovered. These compounds selectively inhibit the activity of COX-2 to a greater extent than the activity of Cox-1. The new COX-2-selective inhibitors are believed to offer advantages that include the capacity to prevent or reduce inflammation while avoiding harmful side effects associated with the inhibition of Cox-1. Thus, cyclooxygenase-2-selective inhibitors have shown great promise for use in therapies—especially in therapies that require extended administration, such as for pain and inflammation control for arthritis. Additional information on the identification of cyclooxygenase-2-selective inhibitors can be found in: (1) Buttgereit, F. et al., Am. J. Med., 110(3 Suppl. 1):13-9 (2001); (2) Osiri, M. et al, Arthritis Care Res., 12(5):351-62 (1999); (3) Buttar, N. S. et al., Mayo Clin. Proc., 75(10):1027-38 (2000); (4) Wollheim, F. A., Current Opin. Rheumatol., 13:193-201 (2001); (5) U.S. Pat. No. 5,434,178 (1,3,5-trisubstituted pyrazole compounds); (6) U.S. Pat. No. 5,476,944 (derivatives of cyclic phenolic thioethers); (7) U.S. Pat. No. 5,643,933 (substituted sulfonylphenylheterocycles); U.S. Pat. No. 5,859,257 (isoxazole compounds); (8) U.S. Pat. No. 5,932,598 (prodrugs of benzenesulfonamide-containing COX-2 inhibitors); (9) U.S. Pat. No. 6,156,781 (substituted pyrazolyl benzenesulfonamides); and (10) U.S. Pat. No. 6,110,960 (for dihydrobenzopyran and related compounds).

The efficacy and side effects of cyclooxygenase-2-selective inhibitors for the treatment of inflammation have been reported. References include: Hillson, J. L. et al., Expert Opin. Pharmacother., 1(5):1053-66 (2000), (for rofecoxib, Vioxx®, Merck & Co., Inc.); Everts, B. et al., Clin. Rheumatol., 19(5):331-43 (2000), (for celecoxib, Celebrex®, Pharmacia Corporation, and rofecoxib); Jamali, F., J. Pharm. Pharm. Sci., 4(1):1-6 (2001), (for celecoxib); U.S. Pat. Nos. 5,521,207 and 5,760,068 (for substituted pyrazolyl benzenesulfonamides); Davies, N. M. et al., Clinical Genetics, Abstr. at http://www.mmhc.com/cg/articles/CG0006/davies.html (for meloxicam, celecoxib, valdecoxib, parecoxib, deracoxib, and rofecoxib); http://www.celebrex.com (for celecoxib); http://www.docguide.com/dg.nsf/PrintPrint/F1F8DDD2D8B0094085256 98F00742187, May 9, 2001 (for etoricoxib, MK-663, Merck & Co., Inc.); Saag, K. et al., Arch. Fam. Med., 9(10):1124-34 (2000), (for rofecoxib); International Patent Publication No. WO 00/24719 (for ABT 963, Abbott Laboratories).

COX-2 inhibitors have also been described for the treatment of cancer (WO98/16227) and for the treatment of tumors (See, EP 927,555, and Rozic et al., Int. J. Cancer, 93(4):497-506 (2001)). Celecoxib®, a selective inhibitor of COX-2, exerted a potent inhibition of fibroblast growth factor-induced corneal angiogenesis in rats. (Masferrer et al., Proc. Am. Assoc. Cancer Research 1999, 40: 396). WO 98/41511 describes 5-(4-sulphunyl-phenyl)-pyridazinone derivatives used for treating cancer. WO 98/41516 describes (methylsulphonyl)phenyl-2-(5H)-furanone derivatives that can be used in the treatment of cancer. Kalgutkar, A. S. et al., Curr. Drug Targets, 2(1):79-106 (2001) suggest that COX-2 selective inhibitors could be used to prevent or treat cancer by affecting tumor viability, growth, and metastasis. Masferrer et al., in Ann. NY Acad. Sci., 889:84-86 (1999) describe COX-2 selective inhibitors as antiangiogenic agents with potential therapeutic utility in several types of cancers. The utility of COX-2 inhibition in clinical cancer prevention was described by Lynch, P. M., in Oncology, 15(3):21-26 (2001), and Watanabe et al., in Biofactors 2000, 12(1-4):129-133 (2000) described the potential of COX-2 selective inhibitors for chemopreventive agents against colon cancer.

Additionally, various combination therapies using COX-2 inhibitors with 25 other selected combination regimens for the treatment of cancer have also been reported. See e.g., FR 27 71 005 (compositions containing a cyclooxygenase-2 inhibitor and N-methyl-d-aspartate (NMDA) antagonist used to treat cancer and other diseases); WO 99/18960 (combination comprising a cyclooxygenase-2 inhibitor and an induced nitric-oxide synthase inhibitor (iNOS) that can be used to treat colorectal and breast cancer); WO 99/13799 (combination of a cyclooxygenase-2 inhibitor and an opioid analgesic); WO 97/36497 (combination comprising a cyclooxygenase-2 inhibitor and a 5-lipoxygenase inhibitor useful in treating cancer); WO 97/29776 (composition comprising a cyclooxygenase-2 inhibitor in combination with a leukotriene B4 receptor antagonist and an immunosuppressive drug); WO 97/29775 (use of a cyclooxygenase-2 inhibitor in combination with a leukotriene A4 hydrolase inhibitor and an immunosuppressive drug); WO 97/29774 (combination of a cyclooxygenase-2 inhibitor and prostaglandin or antiulcer agent useful in treating cancer); WO 97/11701 (combination comprising of a cyclooxygenase-2 inhibitor and a leukotriene B receptor antagonist useful in treating colorectal cancer); WO 96/41645 (combination comprising a cyclooxygenase-2 inhibitor and leukotriene A hydrolase inhibitor); WO 96/03385 (3,4,-Di substituted pyrazole compounds given alone or in combination with NSAIDs, steroids, 5-LO inhibitors, LTB4 antagonists, or LTA4 hydrolase inhibitors for the treatment of cancer); WO 98/47890 (substituted benzopyran derivatives that may be used alone or in combination with other active principles); WO 00/38730 (method of using cyclooxygenase-2 inhibitor and one or more antineoplastic agents as a combination therapy in the treatment of neoplasia); Mann, M. et al., Gastroenterology, 120(7):1713-1719 (2001) (combination treatment with COX-2 and HER-2/neu inhibitors reduced colorectal carcinoma growth).

It is thus desirable to develop novel or improved methods for treatment and prevention of neoplasia.

### SUMMARY OF THE INVENTION

Briefly, therefore the present invention is directed to a novel method for the treatment or prevention of neoplasia disorders in a subject in need of such treatment or prevention, wherein the method comprises administering to the subject a combination comprising a 3-heteroaryl-2-indolinone compound or prodrug thereof and a cyclooxygenase-2 selective inhibitor or prodrug thereof.

In one embodiment, the 3-heteroaryl-2-indolinones of the present invention include compounds having the formula: 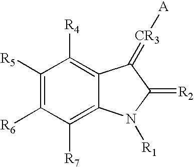
wherein: R₁ is H or alkyl;
R₂ is O or S;
R₃ is hydrogen,
R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂ NRR′, SO₃ R, SR, NO₂, NRR′, OH, CN, C(O)R, OC(O)R, NHC(O)R, (CH₃)ₙ CO₂ R, and CONRR′;
A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, optionally substituted at one or more positions with alkyl, alkoxy, aryl, aryloxy, alkaryl, akaryloxy, halogen, trihalomethyl, S(O)R, SO₂ NRR′, SO3 R, SR, NO₂, NRR′, OH, CN, C(O)R, OC(O)R, NHC(O)R, (CH₂)ₙ CO₂ R, and CONRR′;
n is 0-3;
R is H, alkyl or aryl; and
R′ is H, alkyl or aryl.

The 3-heteroaryl-2-indolinone compounds of the present invention include but are not limited to 3-[(3-Methylpyrrol-2-yl)methylene]-2-indolinone; 3-[(3,4-Dimethylpyrrol-2-yl)methylene]-2-indolinone; 3-[(2-Methylthien-5-yl)methylene]-2-indolinone; 3-[(3-Methylthien-2-yl)methylene]-2-indolinone; 3-{[4-(2-methoxycarbonylethyl)-3-methylpyrrol-5-yl)]methylene}-2-indolinone; 3-[(4,5-Dimethyl-3-ethylpyrrol-2-yl)methylene]-2-indolinone; 3-[(5-Methylimidazol-2-yl)methylene]-2-indolinone; 5-Chloro-3-[(5-methylthien-2-yl)methylene]-2-indolinone; 3-[(3,5-Dimethylpyrrol-2-yl)methylene]-5-nitro-2-indolinone; 3-[(3-(2-carboxyethyl)-4-methylpyrrol-5-yl)methylene]-2-indolinone; 5-Chloro-3-[(3,5-dimethylpyrrol-2-yl)methylene]-2-indolinone; and 3-[(2,4-Dimethylpyrrol-5-yl)methylene]-2-indolinone, and prodrugs thereof.

In a preferred embodiment of the invention, the compound is 3-[(2,4-Dimethylpyrrol-5-yl)methylene]-2-indolinone (SU5416) or a prodrug thereof.

The present invention is also directed to a novel composition for the treatment or prevention of neoplasia comprising a 3-heteroaryl-2-indolinone compound or prodrug thereof and a cyclooxygenase-2 selective inhibitor or prodrug thereof.

The present invention is also directed to a novel pharmaceutical composition comprising a 3-heteroaryl-2-indolinone or prodrug thereof, a cyclooxygenase-2 selective inhibitor or prodrug thereof, and a pharmaceutically-acceptable excipient. Preferably, the 3-heteroaryl-2-indolinone compound is 3-[(2,4-Dimethylpyrrol-5-yl)methylene]-2-indolinone (SU5416) or a prodrug thereof.

The present invention is also directed to a novel kit that is suitable for use in the treatment or prevention of neoplasia, wherein the kit comprises a first dosage form comprising a 3-heteroaryl-2-indolinone compound or prodrug thereof, and a second dosage form comprising a cyclooxygenase-2 selective inhibitor or prodrug thereof, in quantities which comprise a therapeutically effective amount of the compounds for the treatment or prevention of a neoplasia disorder.

### DETAILED DESCRIPTION

“Alkyl” refers to a straight-chain, branched or cyclic saturated aliphatic hydrocarbon. Preferably, the alkyl group has 1 to 12 carbons. More preferably, it is a lower alkyl of from 1 to 7 carbons, more preferably 1 to 4 carbons. Typical alkyl groups include methyl, ethyl, propyl, isopropyl, butyl, isobutyl, tertiary butyl, pentyl, hexyl and the like. The alkyl group may be optionally substituted with one or more substituents selected from the group consisting of hydroxyl, cyano, alkoxy, ═O, ═S, NO₂, halogen, N(CH₃)₂ amino, and SH.

“Alkenyl” refers to a straight-chain, branched or cyclic unsaturated hydrocarbon group containing at least one carbon-carbon double bond. Preferably, the alkenyl group has 1 to 12 carbons. More preferably it is a lower alkenyl of from 1 to 7 carbons, more preferably 1 to 4 carbons. The alkenyl group may be optionally substituted with one or more substituents selected from the group consisting of hydroxyl, cyano, alkoxy, ═O, ═S, NO₂, halogen, N(CH₃)₂, amino, and SH.

“Alkynyl” refers to a straight-chain, branched or cyclic unsaturated hydrocarbon containing at least one carbon-carbon triple bond. Preferably, the alkynyl group has 1 to 12 carbons. More preferably it is a lower alkynyl of from 1 to 7 carbons, more preferably 1 to 4 carbons. The alkynyl group may be optionally substituted with one or more substituents selected from the group consisting of hydroxyl, cyano, alkoxy, ═O, ═S, NO₂, halogen, N(CH₃)₂,amino, and SH.

“Alkoxy” refers to an “-Oalkyl” group.

“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of halogen, trihalomethyl, hydroxyl, SH, OH, NO₂, amine, thioether, cyano, alkoxy, alkyl, and amino.

“Alkaryl” refers to an alkyl that is covalently joined to an aryl group. Preferably, the alkyl is a lower alkyl.

“Carbocyclic aryl” refers to an aryl group wherein the ring atoms are carbon.

“Heterocyclic aryl” refers to an aryl group having from 1 to 3 heteroatoms as ring atoms, the remainder of the ring atoms being carbon. Heteroatoms include oxygen, sulfur, and nitrogen. Thus, heterocyclic aryl groups include furanyl, thienyl, pyridyl, pyrrolyl, N-lower alkyl pyrrolo, pyrimidyl, pyrazinyl, imidazolyl and the like.

“Amide” refers to —C(O)—NH—R, where R is alkyl, aryl, alkylaryl or hydrogen.

“Thioamide” refers to —C(S)—NH—R, where R is alkyl, aryl, alkylaryl or hydrogen.

“Amine” refers to a —N(R′)R″ group, where R′ and R″ are independently selected from the group consisting of alkyl, aryl, and alkylaryl.

“Thioether” refers to —S—R, where R is alkyl, aryl, or alkylaryl.

“Sulfonyl” refers to —S(O)₂—R, where R is aryl, C(CN)═C-aryl, CH₂ CN, alkyaryl, sulfonamide, NH-alkyl, NH-alkylaryl, or NH-aryl.

As used herein, the term “3-heteroaryl-2-indolinone” includes pharmaceutically acceptable salts thereof.

As used herein, 3-heteroaryl-2-indolinone prodrug refers to an agent that is converted into the parent 3-heteroaryl-2-indolinone in vivo. Prodrugs may be easier to administer than the parent drug in some situations. For example, the prodrug may be bioavailable by oral administration but the parent is not, or the prodrug may improve solubility to allow for intravenous administration. A class of prodrugs of 3-heteroaryl-2-indolinones is described in U.S. Pat. No. 6,316,635. References herein to “indolinones”, “oxindoles”, “3-heteroaryl-2-indolinone compounds”, etc. include the prodrugs thereof unless the context precludes It.

The present invention provides methods for the treatment or prevention of neoplasia in a subject in need of such treatment or prevention, wherein the method comprises administering to the subject a combination comprising a 3-heteroaryl-2-indolinone compound or prodrug thereof and a cyclooxygenase-2 selective inhibitor or prodrug thereof.

The methods and combinations of the present invention may be used for the treatment or prevention of neoplasia disorders including acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cnolangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, and Wilm's tumor.

In one embodiment, the 3-heteroaryl-2-indolinone compounds of the present invention include compounds having the formula: 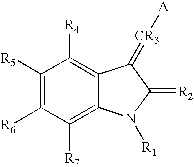
wherein: R₁ is H or alkyl;
R₂ is O or S;
R₃ is hydrogen,
R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂ NRR′, SO₃ R, SR, NO₂, NRR′, OH, CN, C(O)R, OC(O)R, NHC(O)R, (CH₃)ₙ CO₂ R, and CONRR′;
A is a five membered heteroaryl ring selected from the group consisting of thiophene, pyrrole, pyrazole, imidazole, 1,2,3-triazole, 1,2,4-triazole, oxazole, s isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, optionally substituted at one or more positions with alkyl, alkoxy, aryl, aryloxy, alkaryl, akaryloxy, halogen, trihalomethyl, S(O)R, SO₂ NRR′, SO3 R, SR, NO₂, NRR′, OH, CN, C(O)R, OC(O)R, NHC(O)R, (CH₂)ₙ CO₂ R, and CONRR′;
n is 0-3;
R is H, alkyl or aryl; and
R′ is H, alkyl or aryl.

The 3-heteroaryl-2-indolinone compounds of the present invention include but are not limited to 3-[(3-Methylpyrrol-2-yl)methylene]-2-indolinone; 3-[(3,4-Dimethylpyrrol-2-yl)methylene]-2-indolinone; 3-[(2-Methylthien-5-yl)methylene]-2-indolinone; 3-[(3-Methylthien-2-yl)methylene]-2-indolinone; 3-{[4-(2-methoxycarbonylethyl)-3-methylpyrrol-5-yl)]methylene}-2-indolinone; 3-[(4,5-Dimethyl-3-ethylpyrrol-2-yl)methylene]-2-indolinone; 3-[(5-Methylimidazol-2-yl)methylene]-2-indolinone; 5-Chloro-3-[(5-methylthien-2-yl)methylene]-2-indolinone; 3-[(3,5-Dimethylpyrrol-2-yl)methylene]-5-nitro-2-indolinone; 3-[(3-(2-carboxyethyl)-4-methylpyrrol-5-yl)methylene]-2-indolinone; 5-Chloro-3-[(3,5-dimethylpyrrol-2-yl)methylene]-2-indolinone; and 3-[(2,4-Dimethylpyrrol-5-yl)methylene]-2-indolinone, and prodrugs thereof. See U.S. Pat. No. 5,792,783 for a detailed description of 3-heteroaryl-2-indolinone compounds.

In a preferred embodiment of the invention, the 3-heteroaryl-2-indolinone compound is 3-[(2,4-Dimethylpyrrol-5-yl)methylene]-2-indolinone (SU5416) or a prodrug thereof.

In another embodiment, the indolinone combined with the COX-2 inhibitor to treat, prevent or inhibit neoplasia is a pyrrole substituted 2-indolinone, or a pharmaceutically acceptable salt or produg thereof, which modulates the activity of protein kinases. Such indolinones, and methods of providing or preparing them, are fully described in pending U.S. patent application Ser. No. 09/322,297, which has been allowed, and International Publication No. WO 99/61422, which are incorporated herein by reference. In a preferred embodiment, the indolinone is 3-[3,5-dimethyl-4-(2-carboxyethyl)pyrrol-2-ylmethylidene]-2-indolinone(SU-6668).

The chemical formulae of 3-heteroaryl-2-indolinone compounds referred to herein may exhibit the phenomena of tautomerism or structural isomerism. For example, the compounds described herein may adopt a cis or trans conformation about the double bond connecting the S indolinone 3-substituent to the indolinone ring, or may be mixtures of cis and trans isomers. As the formulae drawing within this specification can only represent one possible tautomeric or structural isomeric form, it should be understood that the invention encompasses any tautomeric or structural isomeric form, or mixtures thereof, which possesses the ability to regulate, inhibit and/or modulate tyrosine kinase signal transduction or cell proliferation and is not limited to any one tautomeric or structural isomeric form utilized within the formulae drawing.

In addition to the above-described compounds and their pharmaceutically acceptable salts, the indolinones of the invention include, where applicable, solvated as well as unsolvated forms of the compounds (e.g. hydrated forms) having the ability to regulate and/or modulate cell proliferation.

The 3-heteroaryl-2-indolinone compounds described herein may be prepared by any process known to be applicable to the preparation of chemically-related compounds. Suitable processes are illustrated in the examples. Necessary starting materials may be obtained by standard procedures of organic chemistry.

An individual compound's relevant activity and efficacy as an agent to affect receptor tyrosine kinase mediated signal transduction may be determined using available techniques. Preferentially, a compound is subjected to a series of screens to determine the compound's ability to modulate, regulate and/or inhibit cell proliferation. These screens, in the order in which they are conducted, include biochemical assays, cell growth assays and in vivo experiments.

Preferably, a 3-heteroaryl-2-indolinone compound or prodrug thereof is administered in combination with a COX-2 selective inhibitor or prodrug thereof at a low dose, that is, at a dose lower than has been conventionally used in clinical situations for each of the individual components administered alone.

A benefit of lowering the dose of the compounds, compositions, agents and therapies of the present invention administered to a subject includes a decrease in the incidence of adverse effects associated with higher dosages. For example, by lowering the dosage of a chemotherapeutic agent such as Sugen 5416, a reduction in the frequency and the severity of side effects will result when compared to that observed at higher dosages. Similar benefits are contemplated for use of other 3-heteroaryl-2-indolinone compounds described herein in combination with COX-2 selective inhibitors.

By lowering the incidence of adverse effects, an improvement in the quality of life of a patient undergoing treatment is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects.

The combinations of COX-2 selective inhibitors and 3-heteroaryl-2-indolinone compounds described herein are useful for treating disorders related to unregulated tyrosine kinase signal transduction, including cell proliferative disorders, fibrotic disorders and metabolic disorders. The ability to use 3-heteroaryl-2-indolinones to treat such diseases stems from the fact that these compounds regulate, modulate and/or inhibit tyrosine kinase signal transduction by affecting the enzymatic activity of the receptor tyrosine kinases (RTKs) and/or the non-receptor tyrosine kinases and interfering with the signal transduced by such proteins.

Tyrosine kinase signal transduction plays an important role in cell proliferation, differentiation and metabolism. Abnormal cell proliferation may result in a wide array of disorders and diseases, including the development of neoplasia such as carcinoma, sarcoma, leukemia, glioblastoma, hemangioma, psoriasis, arteriosclerosis, arthritis and diabetic retinopathy (or other disorders related to uncontrolled angiogenesis and/or vasculogenesis). Thus, the combinations disclosed herein containing 3-heteroaryl-2-indolinone compounds are useful, e.g., in treating diseases resulting from abnormal tyrosine kinase signal transduction.

Cell proliferative disorders which can be treated or further studied by the present invention, include, in addition to cancers, blood vessel proliferative disorders and mesangial cell proliferative disorders.

Blood vessel proliferative disorders refer to angiogenic and vasculogenic disorders generally resulting in abnormal proliferation of blood vessels. The formation and spreading of blood vessels, or vasculogenesis and angiogenesis, respectively, play important roles in a variety of physiological processes such as embryonic development, corpus luteum formation, wound healing and organ regeneration. They also play a pivotal role in cancer development. Other examples of blood vessel proliferation disorders include arthritis, where new capillary blood vessels invade the joint and destroy cartilage, and ocular diseases, like diabetic retinopathy, where new capillaries in the retina invade the vitreous, bleed and cause blindness. Conversely, disorders related to the shrinkage, contraction or closing of blood vessels, such as restenosis, are also implicated.

Fibrotic disorders refer to the abnormal formation of extracellular matrix. Examples of fibrotic disorders include hepatic cirrhosis and mesangial cell proliferative disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis. Other fibrotic disorders implicated include atherosclerosis (see, below).

Mesangial cell proliferative disorders refer to disorders brought about by abnormal proliferation of mesangial cells. Mesangial proliferative disorders include various human renal diseases, such as glomerulonephritis, diabetic nephropathy, malignant nephrosclerosis, thrombotic microangiopathy syndromes, transplant rejection, and glomerulopathies. The PDGF-R has been implicated in the maintenance of mesangial cell proliferation. Floege et al., 1993, Kidney International 43:47S-54S.

PTKs have been associated with such cell proliferative disorders. For example, some members of the RTK family have been associated with the development of cancer. Some of these receptors, like the EGFR (Tuzi et al., 1991, Br. J. Cancer 63:227-233; Torp et al., 1992, APMIS 100:713-719) HER2/neu (Slamon et al., 1989, Science 244:707-712) and the PDGF-R (Kumabe et al., 1992, Oncogene 7:627-633) are overexpressed in many tumors and/or persistently activated by autocrine loops. In fact, in the most common and severe cancers, these receptor overexpressions (Akbasak and Suner-Akbasak et al., 1992, J. Neurol. Sci. 111:119-133; Dickson et al., 1992, Cancer Treatment Res. 61:249-273; Korc et al., 1992, J. Clin. Invest. 90:1352-1360) and autocrine loops (Lee and Donoghue, 1992, J. Cell. Biol. 118:1057-1070; Korc et al., supra; Akbasak and Suner-Akbasak et al., supra) have been demonstrated. For example, the EGFR receptor has been associated with squamous cell carcinoma, astrocytoma, glioblastoma, head and neck cancer, lung cancer and bladder cancer. HER2 has been associated with breast, ovarian, gastric, lung, pancreas and bladder cancer. The PDGF-R has been associated with glioblastoma, lung, ovarian, melanoma and prostate cancer. The RTK c-met has been generally associated with hepatocarcinogenesis and thus hepatocellular carcinoma. Additionally, c-met has been linked to malignant tumor formation. More specifically, the RTK c-met has been associated with, among other cancers, colorectal, thyroid, pancreatic and gastric carcinoma, leukemia and lymphoma. Additionally, over-expression of the c-met gene has been detected in patients with Hodgkins disease, Burkitts disease, and the lymphoma cell line.

The IGF-IR, in addition to being implicated in nutritional support and in type-II diabetes, has also been associated with several types of cancers. For example, IGF-I has been implicated as an autocrine growth stimulator for several tumor types, e.g. human breast cancer carcinoma cells (Arteaga et al., 1989, J. Clin. Invest. 84:1418-1423) and small lung tumor cells (Macauley et al., 1990, Cancer Res. 50:2511-2517). In addition, IGF-I, integrally involved in the normal growth and differentiation of the nervous system, appears to be an autocrine stimulator of human gliomas. Sandberg-Nordqvist et al., 1993, Cancer Res. 53:2475-2478. The importance of the IGF-IR and its ligands in cell proliferation is further supported by the fact that many cell types in culture (fibroblasts, epithelial cells, smooth muscle cells, T-lymphocytes, myeloid cells, chondrocytes, osteoblasts, the stem cells of the bone marrow) are stimulated to grow by IGF-I. Goldring and Goldring, 1991, Eukaryotic Gene Expression 1:301-326. In a series of recent publications, Baserga even suggests that IGF-I-R plays a central role in the mechanisms of transformation and, as such, could be a preferred target for therapeutic interventions for a broad spectrum of human malignancies. Baserga, 1995, Cancer Res. 55:249-252; Baserga, 1994, Cell 79:927-930; Coppola et al., 1994, Mol . Cell. Biol. 14:4588-4595.

The association between abnormalities in RTKs and disease are not only restricted to cancer, however. For example, RTKs have been associated with metabolic diseases like psoriasis, diabetes mellitus, wound healing, inflammation, and neurodegenerative diseases. For example, the EGF-R is indicated in corneal and dermal wound healing. Defects in the Insulin-R and the IGF-IR are indicated in type-II diabetes mellitus. A more complete correlation between specific RTKs and their therapeutic indications is set forth in Plowman et al., 1994, DN&P 7:334-339.

Not only receptor type tyrosine kinases, but also many cellular tyrosine kinases (CTKs) including src, abl, fps, yes, fyn, lyn, lck, blk, hck, fgr, yrk (reviewed by Bolen et al., 1992, FASEB J. 6:3403-3409) are involved in the proliferative and metabolic signal transduction pathway and thus in indications of the present invention. For example, mutated src (v-src) has been demonstrated as an oncoprotein (pp60^{v-src}) in chicken. Moreover, its cellular homolog, the proto-oncogene pp60^{c-src} transmits oncogenic signals of many receptors. For example, overexpression of EGF-R or HER2/neu in tumors leads to the constitutive activation of pp60^{c-src}, which is characteristic for the malignant cell but absent from the normal cell. On the other hand, mice deficient for the expression of c-src exhibit an osteopetrotic phenotype, indicating a key participation of c-src in osteoclast function and a possible involvement in related disorders. Similarly, Zap 70 is implicated in T-cell signaling.

Furthermore, the identification of CTK modulating compounds to augment or even synergize with RTK aimed blockers is an aspect of the present invention.

Finally, both RTKs and non-receptor type kinases have been connected to hyperimmune disorders.

Thus, in addition to being used to treat neoplasia, the combination therapy of the present invention may be used to treat diseases such as blood vessel proliferative disorders, fibrotic disorders, mesangial cell proliferative disorders and metabolic diseases.

As used herein, the term “cyclooxygenase-2 inhibitor” embraces compounds which selectively inhibit cyclooxygenase-2 over cyclooxygenase-1, and also includes pharmaceutically acceptable salts or esters of those compounds.

In practice, the selectivity of a COX-2 inhibitor varies depending upon the condition under which the test is performed and on the inhibitors being tested. However, for the purposes of this specification, the selectivity of a COX-2 inhibitor can be measured as a ratio of the in vitro or in vivo IC₅₀ value for inhibition of Cox-1, divided by the IC₅₀ value for inhibition of COX-2 (Cox-1 IC₅₀/COX-2 IC₅₀). A COX-2 selective inhibitor is any inhibitor for which the ratio of Cox-1 IC₅₀ to COX-2 IC₅₀ is greater than 1, preferably greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity.

Preferred cyclooxygenase-2 selective inhibitors of the present invention have a cyclooxygenase-2 IC₅₀ of less than about 1 μM, more preferred of about 0.5 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to COX-2 selective inhibitors, the term “prodrug” refers to a chemical compound that can be converted into an active COX-2 selective inhibitor by metabolic or simple chemical processes within the body of the subject. One example of a prodrug for a COX-2 selective inhibitor is parecoxib, which is a therapeutically effective prodrug of the tricyclic cyclooxygenase-2 selective inhibitor valdecoxib. An example of a preferred COX-2 selective inhibitor prodrug is parecoxib sodium. A class of prodrugs of COX-2 inhibitors is described in U.S. Pat. No. 5,932,598. References herein to “cyclooxygenase-2 selective inhibitors”, “COX-2 selective inhibitors”, etc. include prodrugs thereof unless the context precludes it.

In one embodiment, COX-2 inhibitors used in the methods and compositions described herein are selected from the group consisting of substituted benzothiopyrans, dihydroquinolines, or dihydronaphthalenes having the general Formula (I):

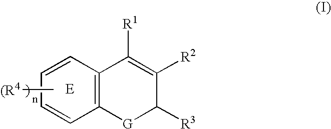

or an isomer, a pharmaceutically acceptable salt, an ester, or a prodrug thereof,

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with carbon atoms to which it is attached and the remainder of the ring E forms a naphthyl radical;

or an isomer, a pharmaceutically acceptable salt, an ester, or a prodrug thereof,

In another embodiment, the COX-2 inhibitors used herein have the general Formula (II): 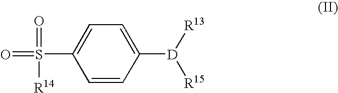
or an isomer, a pharmaceutically acceptable salt, an ester, or a prodrug thereof,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

According to another embodiment, the present invention is also directed to novel compositions for the treatment, prevention or inhibition of neoplasia disorders comprising administering to a subject in need thereof, a cyclooxygenase-2 (COX-2) inhibitor in a first amount and 3-heteroaryl-2-indolinone in a second amount, wherein said first amount together with said second amount is a therapeutically effective amount of said COX-2 inhibitor and t3-heteroaryl-2-indolinone, and wherein said COX-2 inhibitor comprises a phenylacetic acid derivative represented by the general Formula (III): 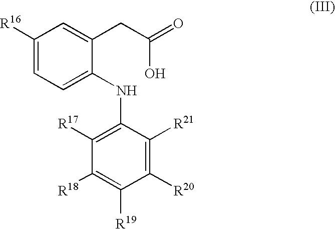
or an isomer, a pharmaceutically acceptable salt, an ester, or a prodrug thereof,

wherein:

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R ²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

In another embodiment, the COX-2 inhibitors useful in the compositions and methods of the present invention are represented by Formula (IV): 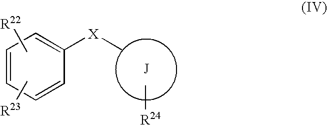
or an isomer, a pharmaceutically acceptable salt, an ester, or a prodrug thereof,

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the COX-2 inhibitors described herein have structural Formula (V): 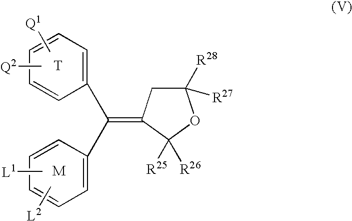
or an isomer, a pharmaceutically acceptable salt, an ester, or a prodrug thereof,

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

The cyclooxygenase-2 selective inhibitor of the present invention can be, for example, the COX-2 selective inhibitor meloxicam, Formula B-0 (CAS registry number 71125-38-7), or a pharmaceutically acceptable salt or prodrug thereof.

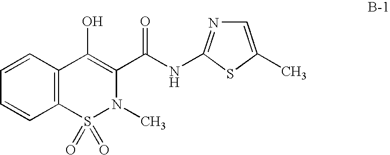

In another embodiment of the invention the cyclooxygenase-2 selective inhibitor can be the COX-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

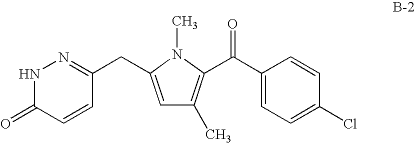

The cyclooxygenase-2 selective inhibitor of the present invention can be, for example, the COX-2 selective inhibitor [2-(2,4-Dichloro-6-ethyl-3,5-dimethyl-phenylamino)-5-propyl-phenyl]-acetic acid, having Formula B-1, or an isomer or pharmaceutically acceptable salt, ester, or prodrug thereof.

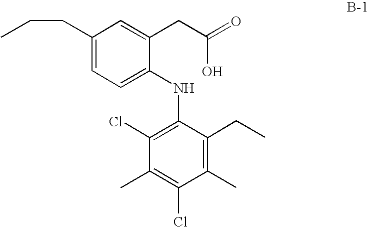

In a preferred embodiment of the invention the cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having a structure shown by general Formula I, shown herein, and possessing, by way of example and not limitation, the structures disclosed in is Table 1, including the diastereomers, enantiomers, racemates, tautomers, salts, esters, amides and prodrugs thereof.

Furthermore, benzopyran COX-2 selective inhibitors useful in the practice of the present invention are described in U.S. Pat. Nos. 6,034,256 and 6,077,850.

The cyclooxygenase-2 selective inhibitor may also be a compound of Formula (I), or an isomer, a pharmaceutically acceptable salt, ester, or prodrug thereof; wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

The cyclooxygenase-2 selective inhibitor used in connection with the method(s) of the present invention can also be a compound having the structure of Formula (I) or an isomer, a pharmaceutically acceptable salt, ester, or prodrug thereof:

wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6- membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia): 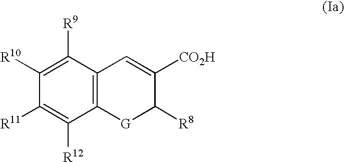
or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

The present invention is also directed to a novel method for the treatment of neoplasia disorders comprising administering to a subject in need thereof a therapeutically effective amount of a cyclooxygenase-2 selective inhibitor comprising BMS-347070 (B-74), ABT 963 (B-25), NS-398 (B-26), L-745337 (B-214), RWJ-63556 (B-215), or L-784512 (B-216).

Of the COX-2 inhibitors, those listed in Table 1 are chromene COX-2 inhibitors as indicated below:

TABLE 1
Examples of Chromene COX-2 Selective Inhibitors

| No. | Structure (chromene COX-2 Inhibitor) |
|---|---|
| B-3 | 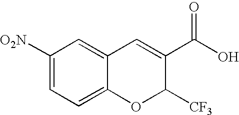 |
| | 6-Nitro-2-trifluoromethyl-2H-1- |
| | benzopyran-3-carboxylic acid |
| B-4 | 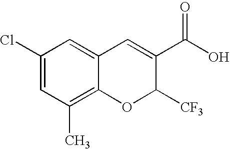 |
| | 6-Chloro-8-methyl-2-trifluoromethyl- |
| | 2H-1-benzopyran-3-carboxylic acid |
| B-5 | 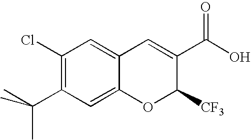 |
| | ((S)-6-Chloro-7-(1,1-dimethylethyl)-2-(tri- |
| | fluoromethyl-2H-1-benzopyran-3-carboxylic acid |
| B-6 | 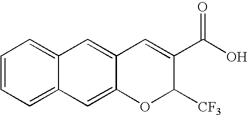 |
| | 2-Trifluoromethyl-2H-naphtho[2,3-b] |
| | pyran-3-carboxylic acid |
| B-7 | 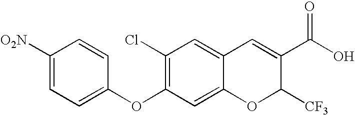 |
| | 6-Chloro-7-(4-nitrophenoxy)-2-(trifluoromethyl)-2H-1- |
| | benzopyran-3-carboxylic acid |
| B-8 | 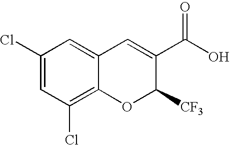 |
| | ((S)-6,8-Dichloro-2-(trifluoromethyl)- |
| | 2H-1-benzopyran-3-carboxylic acid |
| B-9 | 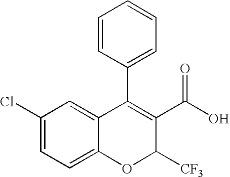 |
| | 6-Chloro-2-(trifluoromethyl)-4-phenyl-2H- |
| | 1-benzopyran-3-carboxylic acid |
| B-10 | 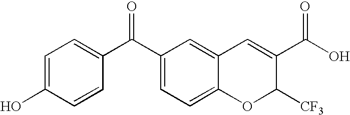 |
| | 6-(4-Hydroxybenzoyl)-2-(trifluoromethyl)- |
| | 2H-1-benzopyran-3-carboxylic acid |
| B-11 | 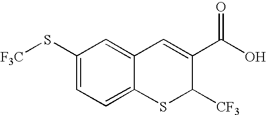 |
| | 2-(Trifluoromethyl)-6-[(trifluoromethyl) thio]- |
| | 2H-1-benzothiopyran-3-carboxylic acid |
| B-12 | 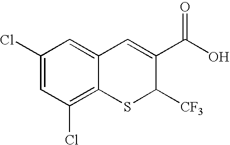 |
| | 6,8-Dichloro-2-trifluoromethyl-2H-1- |
| | benzothiopyran-3-carboxylic acid |
| B-13 | 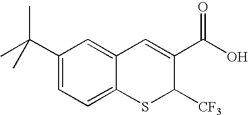 |
| | 6-(1,1-Dimethylethyl)-2-(trifluoromethyl)- |
| | 2H-1-benzothiopyran-3-carboxylic acid |
| B-14 | 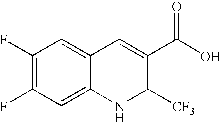 |
| | 6,7-Difluoro-1,2-dihydro-2-(trifluoro |
| | methyl)-3-quinolinecarboxylic acid |
| B-15 | 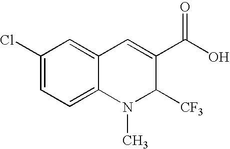 |
| | 6-Chloro-1,2-dihydro-1-methyl-2-(trifluoro |
| | methyl)-3-quinolinecarboxylic acid |
| B-16 | 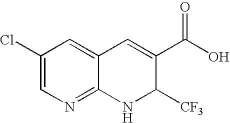 |
| | 6-Chloro-2-(trifluoromethyl)-1,2-dihydro |
| | [1,8]naphthyridine-3-carboxylic acid |
| B-17 | 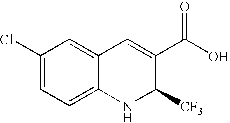 |
| | ((S)-6-Chloro-1,2-dihydro-2-(trifluoro |
| | methyl)-3-quinolinecarboxylic acid |

In a further preferred embodiment of the invention the cyclooxygenase inhibitor, when used in combination with indolinone can be selected from the class of tricyclic cyclooxygenase-2 selective inhibitors represented by the general structure of Formula (II): 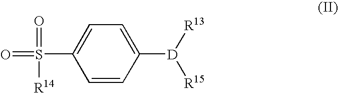
or an isomer, a pharmaceutically acceptable salt, ester, or prodrug thereof,

wherein:

D is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In a still more preferred embodiment of the invention, the tricyclic cyclooxygenase-2 selective inhibitor(s), for use in connection with the method(s) of the present invention and in combination with an indolinone are represented by the above Formula (II) and are selected from the group of compounds, illustrated in Table 2, consisting of celecoxib (B-18), valdecoxib (B-19), deracoxib (B-20), rofecoxib (B-21), etoricoxib (MK-663; B-22), JTE-522 (B-23), or an isomer, a pharmaceutically acceptable salt, ester, or prodrug thereof.

TABLE 2
Examples of Tricyclic COX-2 Selective Inhibitors

| No. | Structure (Tricyclic COX-2 Inhibitors) |
|---|---|
| B-18 | 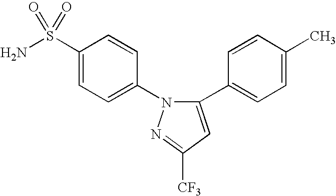 |
| | celecoxib |
| B-19 | 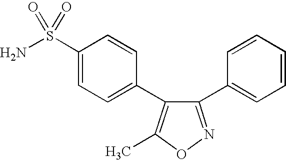 |
| | valdecoxib |
| B-20 | 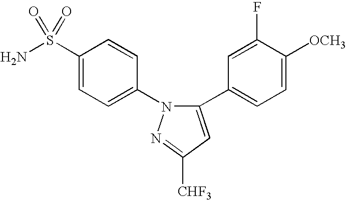 |
| | deracoxib |
| B-21 | 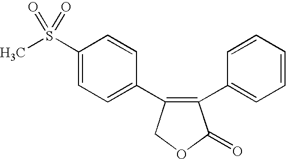 |
| | rofecoxib |
| B-22 | 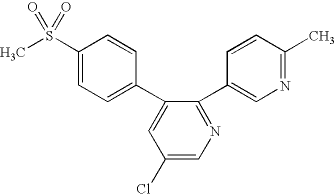 |
| | etoricoxib |
| B-23 | 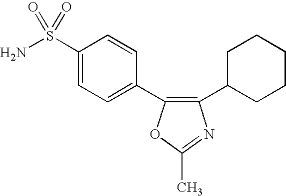 |
| | JTE-522 |

In an even more preferred embodiment of the invention, the COX-2 selective inhibitor, when used in combination with an indolinone is selected from the group consisting of celecoxib, rofecoxib and etoricoxib.

In another preferred embodiment of the invention, parecoxib, (B-24), which is a therapeutically effective prodrug of the tricyclic cyclooxygenase-2 selective inhibitor valdecoxib, (B-19), may be advantageously employed as a source of a cyclooxygenase inhibitor (See, e.g., U.S. Pat. No. 5,932,598) in connection with the method(s) in the present invention.

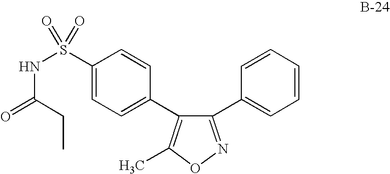

A preferred form of parecoxib is sodium parecoxib.

In another preferred embodiment of the invention, the compound ABT-963 having the formula (B-25) that has been previously described in International Publication number WO 00/24719, is another tricyclic cyclooxygenase-2 selective inhibitor which may be advantageously employed in connection with the method(s) of the present invention.

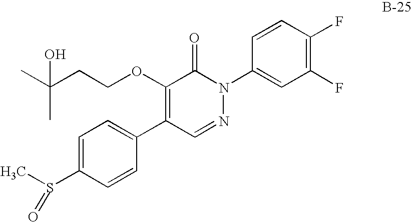

Another preferred cyclooxygenase-2 selective inhibitor that is useful in connection with the method(s) of the present invention is N-(2-cyclohexyloxynitrophenyl)-methane sulfonamide (NS-398)—having a structure is shown below as B-26. Applications of this compound have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (Jun. 06, 2001); and Iwata, K. et al., in Jpn. J. Pharmacol., 75(2):191-194 (1997).

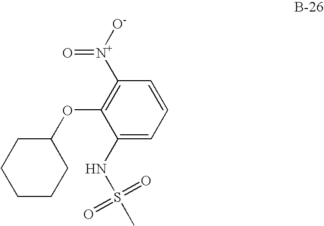

Other compounds that are useful for the cyclooxygenase-2 selective inhibitor in connection with the method(s) of the present invention include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1-methylethyl)-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-trifluoromethyl-2H-1-benzopyran-3-carboxylic acid (B-39);
7-(1-methylethyl)-2-trifluoromethyl-2H-1-benzopyran-3-carboxylic acid (B-40);
7-phenyl-2-trifluoromethyl-2H-1-benzopyran-3-carboxylic acid (B-41);
6-chloro-7-ethyl-2-trifluoromethyl-2H-1-benzopyran-3-carboxylic acid (B-42);
6-chloro-8-ethyl-2-trifluoromethyl-2H-1-benzopyran-3-carboxylic acid (B-43);
6-chloro-7-phenyl-2-trifluoromethyl-2H-1-benzopyran-3-carboxylic acid (B-44);
6,7-dichloro-2-trifluoromethyl-2H-1-benzopyran-3-carboxylic acid (B-45);
6,8-dichloro-2-trifluoromethyl-2H-1-benzopyran-3-carboxylic acid (B-46);
6-chloro-8-methyl-2-trifluoromethyl-2H-1-benzopyran-3-carboxylic acid (B-47);
8-chloro-6-methyl-2-trifluoromethyl-2H-1-benzopyran-3-carboxylic acid (B-48)
8-chloro-6-methoxy-2-trifluoromethyl-2H-1-benzopyran-3-carboxylic acid (B-49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic acid (B-71);
7-(1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-Chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a)pyridine (B-75);
5,5-dimethyl-4-(4-methylsulfonyl)phenyl-3-phenyl-2-(5H)-furanone (B-76);
5-(4-fluorophenyl)-1-[4-(methylsulfonyl)phenyl]-3-(trifluoromethyl)pyrazole (B-77);
4-(4-fluorophenyl)-5-[4-(methylsulfonyl)phenyl]-1-phenyl-3-(trifluoromethyl)pyrazole (B-78);
4-(5-(4-chlorophenyl)-3-(4-methoxyphenyl)-1H-pyrazol-1-yl)benzenesulfonamide (B-79);
4-(3,5-bis(4-methylphenyl)-1H-pyrazol-1-yl)benzenesulfonamide (B-80);
4-(5-(4-chlorophenyl)-3-phenyl-1H-pyrazol-1-yl)benzenesulfonamide (B-81);
4-(3,5-bis(4-methoxyphenyl)-1H-pyrazol-1-yl)benzenesulfonamide (B-82);
4-(5-(4-chlorophenyl)-3-(4-methylphenyl)-1H-pyrazol-1-yl)benzenesulfonamide (B-83);
4-(5-(4-chlorophenyl)-3-(4-nitrophenyl)-1H-pyrazol-1-yl)benzenesulfonamide (B-84);
4-(5-(4-chlorophenyl)-3-(5-chloro-2-thienyl)-1H-pyrazol-1-yl)benzenesulfonamide (B-85);
4-(4-chloro-3,5-diphenyl-1H-pyrazol-1-yl)benzenesulfonamide (B-86);
4-[5-(4-chlorophenyl)-3-(trifluoromethyl)-1H-pyrazol-1-yl]benzenesulfonamide (B-87);
4-[5-phenyl-3-(trifluoromethyl)-1H-pyrazol-1-yl]benzenesulfonamide (B-88);
4-[5-(4-fluorophenyl)-3-(trifluoromethyl)-1H-pyrazol-1-yl]benzenesulfonamide (B-89);
4-[5-(4-methoxyphenyl)-3-(trifluoromethyl)-1H-pyrazol-1-yl]benzenesulfonamide (B-90);
4-[5-(4-chlorophenyl)-3-(difluoromethyl)-1H-pyrazol-1-yl]benzenesulfonamide (B-91);
4-[5-(4-methylphenyl)-3-(trifluoromethyl)-1H-pyrazol-1-yl]benzenesulfonamide (B-92);
4-[4-chloro-5-(4-chlorophenyl)-3-(trifluoromethyl)-1H-pyrazol-1-yl]benzenesulfonamide (B-93);
4-[3-(difluoromethyl)-5-(4-methylphenyl)-1H-pyrazol-1-yl]benzenesulfonamide (B-94);
4-[3-(difluoromethyl)-5-phenyl-1H-pyrazol-1-yl]benzenesulfonamide (B-95);
4-[3-(difluoromethyl)-5-(4-methoxyphenyl)-1H-pyrazol-1-yl]benzenesulfonamide (B-96);
4-[3-cyano-5-(4-fluorophenyl)-1H-pyrazol-1-yl]benzenesulfonamide (B-97);
4-[3-(difluoromethyl)-5-(3-fluoro-4-methoxyphenyl)-1H-pyrazol-1-yl]benzenesulfonamide (B-98);
4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);
4-[5-(4-chlorophenyl)-3-(hydroxymethyl)-1H-pyrazol-1-yl]benzenesulfonamide (B-101);
4-[5-(4-(N,N-dimethylamino)phenyl)-3-(trifluoromethyl)-1H-pyrazol-1-yl]benzenesulfonamide (B-102);
5-(4-fluorophenyl)-6-[4-(methylsulfonyl)phenyl]spiro[2.4]hept-5-ene (B-103);
4-[6-(4-fluorophenyl)spiro[2.4]hept-5-en-5-yl]benzenesulfonamide (B-104);
6-(4-fluorophenyl)-7-[4-(methylsulfonyl)phenyl]spiro[3.4]oct-6-ene (B-105);
5-(3-chloro-4-methoxyphenyl)-6-[4-(methylsulfonyl)phenyl]spiro[2.4]hept-5-ene (B-106);
4-[6-(3-chloro-4-methoxyphenyl)spiro[2.4]hept-5-en-5-yl]benzenesulfonamide (B-107);
5-(3,5-dichloro-4-methoxyphenyl)-6-[4-(methylsulfonyl)phenyl]spiro[2.4]hept-5-ene (B-108);
5-(3-chloro-4-fluorophenyl)-6-[4-(methylsulfonyl)phenyl]spiro[2.4]hept-5-ene (B-109);
4-[6-(3,4-dichlorophenyl)spiro[2.4]hept-5-en-5-yl]benzenesulfonamide (B-110);
2-(3-chloro-4-fluorophenyl)-4-(4-fluorophenyl)-5-(4-methylsulfonylphenyl)thiazole (B-111);
2-(2-chlorophenyl)-4-(4-fluorophenyl)-5-(4-methylsulfonylphenyl)thiazole (B-112);
5-(4-fluorophenyl)-4-(4-methylsulfonylphenyl)-2-methylthiazole (B-113);
4-(4-fluorophenyl)-5-(4-methylsulfonylphenyl)-2-trifluoromethylthiazole (B-114);
4-(4-fluorophenyl)-5-(4-methylsulfonylphenyl)-2-(2-thienyl)thiazole (B-115);
4-(4-fluorophenyl)-5-(4-methylsulfonylphenyl)-2-benzylaminothiazole (B-116);
4-(4-fluorophenyl)-5-(4-methylsulfonylphenyl)-2-(1-propylamino)thiazole (B-117);
2-[(3,5-dichlorophenoxy)methyl)-4-(4-fluorophenyl)-5-[4-(methylsulfonyl)phenyl]thiazole (B-118);
5-(4-fluorophenyl)-4-(4-methylsulfonylphenyl)-2-trifluoromethylthiazole (B-119);
1-methylsulfonyl-4-[1,1-dimethyl-4-(4-fluorophenyl)cyclopenta-2,4-dien-3-yl]benzene (B-120);
4-[4-(4-fluorophenyl)-1,1-dimethylcyclopenta-2,4-dien-3-yl]benzenesulfonamide (B-121);
5-(4-fluorophenyl)-6-[4-(methylsulfonyl)phenyl]spiro[2.4]hepta-4,6-diene (B-122);
4-[6-(4-fluorophenyl)spiro[2.4]hepta4,6-dien-5-yl]benzenesulfonamide (B-123);
6-(4-fluorophenyl)-2-methoxy-5-[4-(methylsulfonyl)phenyl]-pyridine-3-carbonitrile (B-124);
2-bromo-6-(4-fluorophenyl)-5-[4(methylsulfonyl)phenyl]-pyridine-3-carbonitrile (B-125);
6-(4-fluorophenyl)-5-[4-(methylsulfonyl)phenyl]-2-phenyl-pyridine-3-carbonitrile (B-126);
4-[2-(4-methylpyridin-2-yl)-4-(trifluoromethyl)-1H-imidazol-1-yl]benzenesulfonamide (B-127);
4-[2-(5-methylpyridin-3-yl)-4-(trifluoromethyl)-1H-imidazol-1-yl]benzenesulfonamide (B-128);
4-[2-(2-methylpyridin-3-yl)-4-(trifluoromethyl)-1H-imidazol-1-yl]benzenesulfonamide (B-129);
3-[1-[4-(methylsulfonyl)phenyl]-4-(trifluoromethyl)-1H-imidazol-2-yl]pyridine (B-130);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
4-[2-(6-methylpyridin-3-yl)-4-(trifluoromethyl)-1H-imidazol-1-yl]benzenesulfonamide (B-134);
2-(3,4-difluorophenyl)-1-[4-(methylsulfonyl)phenyl]-4-(trifluoromethyl)-1H-imidazole (B-135);
4-[2-(4-methylphenyl)-4-(trifluoromethyl)-1H-imidazol-1-yl]benzenesulfonamide (B-136);
2-(4-chlorophenyl)-1-[4-(methylsulfonyl)phenyl]-4-methyl-1H-imidazole (B-137);
2-(4-chlorophenyl)-1-[4-(methylsulfonyl)phenyl]-4-phenyl-1H-imidazole (B-138);
2-(4-chlorophenyl)-4-(4-fluorophenyl)-1-[4-(methylsulfonyl)phenyl]-1H-imidazole (B-139);
2-(3-fluoro-4-methoxyphenyl)-1-[4-(methylsulfonyl)phenyl-4-(trifluoromethyl)-1H-imidazole (B-140);
1-[4-(methylsulfonyl)phenyl]-2-phenyl-4-trifluoromethyl-1H-imidazole (B-141);
2-(4-methylphenyl)-1-[4-(methylsulfonyl)phenyl]-4-trifluoromethyl-1H-imidazole (B-142);
4-[2-(3-chloro-4-methylphenyl)-4-(trifluoromethyl)-1H-imidazol-1-yl]benzenesulfonamide (B-143);
2-(3-fluoro-5-methylphenyl)-1-[4-(methylsulfonyl)phenyl]4-(trifluoromethyl)-1H-imidazole (B-144);
4-[2-(3-fluoro-5-methylphenyl)-4-(trifluoromethyl)-1H-imidazol-1-yl]benzenesulfonamide (B-145);
2-(3-methylphenyl)-1-[4-(methylsulfonyl)phenyl]-4-trifluoromethyl-1H-imidazole (B-146);
4-[2-(3-methylphenyl)-4-trifluoromethyl-1H-imidazol-1-yl]benzenesulfonamide (B-147);
1-[4-(methylsulfonyl)phenyl]-2-(3-chlorophenyl)-4-trifluoromethyl-1H-imidazole (B-148);
4-[2-(3-chlorophenyl)-4-trifluoromethyl-1H-imidazol-1-yl]benzenesulfonamide (B-149);
4-[2-phenyl-4-trifluoromethyl-1H-imidazol-1-yl]benzenesulfonamide (B-150);
4-[2-(4-methoxy-3-chlorophenyl)-4-trifluoromethyl-1H-imidazol-1-yl]benzenesulfonamide (B-151);
1-allyl-4-(4-fluorophenyl)-3-[4-(methylsulfonyl)phenyl]-5-(trifluoromethyl)-1H-pyrazole (B-152);
4-[1-ethyl-4-(4-fluorophenyl)-5-(trifluoromethyl)-1H-pyrazol-3-yl]benzenesulfonamide (B-153);
N-phenyl-[4-(4-fluorophenyl)-3-[4-(methylsulfonyl)phenyl]-5-(trifluoromethyl)-1H-pyrazol-1-yl]acetamide (B-154);
ethyl [4-(4-fluorophenyl)-3-[4-(methylsulfonyl)phenyl]-5-(trifluoromethyl)-1H-pyrazol-1-yl]acetate (B-155);
4-(4-fluorophenyl)-3-[4-(methylsulfonyl)phenyl]-1-(2-phenylethyl)-1H-pyrazole (B-156);
4-(4-fluorophenyl)-3-[4-(methylsulfonyl)phenyl]-1-(2-phenylethyl)-5-(trifluoromethyl)pyrazole (B-157);
1-ethyl-4-(4-fluorophenyl)-3-[4-(methylsulfonyl)phenyl]-5-(trifluoromethyl)-1H-pyrazole (B-158);
5-(4-fluorophenyl)-4-(4-methylsulfonylphenyl)-2-trifluoromethyl-1H-imidazole (B-159);
4-[4-(methylsulfonyl)phenyl]-5-(2-thiophenyl)-2-(trifluoromethyl)-1H-imidazole (B-160);
5-(4-fluorophenyl)-2-methoxy-4-[4-(methylsulfonyl)phenyl]-6-(trifluoromethyl)pyridine (B-161);
2-ethoxy-5-(4-fluorophenyl)-4-[4-(methylsulfonyl)phenyl]-6-(trifluoromethyl)pyridine (B-162);
5- (4-fluorophenyl)-4-[4-(methylsulfonyl)phenyl]-2-(2-propynyloxy)-6-(trifluoromethyl)pyridine (B-163);
2-bromo-5-(4-fluorophenyl)-4-[4-(methylsulfonyl)phenyl]-6-(trifluoromethyl)pyridine (B-164);
4-[2-(3-chloro-4-methoxyphenyl)-4,5-difluorophenyl]benzenesulfonamide (B-165);
1-(4-fluorophenyl)-2-[4-(methylsulfonyl)phenyl]benzene (B-166);
5-difluoromethyl-4-(4-methylsulfonylphenyl)-3-phenylisoxazole (B-167);
4-[3-ethyl-5-phenylisoxazol-4-yl]benzenesulfonamide (B-168);
4-[5-difluoromethyl-3-phenylisoxazol-4-yl]benzenesulfonamide (B-169);
4-[5-hydroxymethyl-3-phenylisoxazol-4-yl]benzenesulfonamide (B-170);
4-[5-methyl-3-phenyl-isoxazol-4-yl]benzenesulfonamide (B-171);
1-[2-(4-fluorophenyl)cyclopenten-1-yl]-4-(methylsulfonyl)benzene (B-172);
1-[2-(4-fluoro-2-methylphenyl)cyclopenten-1-yl]-4-(methylsulfonyl)benzene (B-173);
1-[2-(4-chlorophenyl)cyclopenten-1-yl]-4-(methylsulfonyl)benzene (B-174);
1-[2-(2,4-dichlorophenyl)cyclopenten-1-yl]-4-(methylsulfonyl)benzene (B-175);
1-[2-(4-trifluoromethylphenyl)cyclopenten-1-yl]-4-(methylsulfonyl)benzene (B-176);
1-[2-(4-methylthiophenyl)cyclopenten-1-yl]-4-(methylsulfonyl)benzene (B-177);
1-[2-(4-fluorophenyl)-4,4-dimethylcyclopenten-1-yl]-4-(methylsulfonyl)benzene (B-178);
4-[2-(4-fluorophenyl)-4,4-dimethylcyclopenten-1-yl]benzenesulfonamide (B-179);
1-[2-(4-chlorophenyl)-4,4-dimethylcyclopenten-1-yl]-4-(methylsulfonyl)benzene (B-180);
4-[2-(4-chlorophenyl)-4,4-dimethylcyclopenten-1-yl]benzenesulfonamide (B-181);
4-[2-(4-fluorophenyl)cyclopenten-1-yl]benzenesulfonamide (B-182);
4-[2-(4-chlorophenyl)cyclopenten-1-yl]benzenesulfonamide (B-183);
1-[2-(4-methoxyphenyl)cyclopenten-1-yl]-4-(methylsulfonyl)benzene (B-184);
1-[2-(2,3-difluorophenyl)cyclopenten-1-yl]-4-(methylsulfonyl)benzene (B-185);
4-[2-(3-fluoro-4-methoxyphenyl)cyclopenten-1-yl]benzenesulfonamide (B-186);
1-[2-(3-chloro-4-methoxyphenyl)cyclopenten-1-yl]-4-(methylsulfonyl)benzene (B-187);
4-[2-(3-chloro-4-fluorophenyl)cyclopenten-1-yl]benzenesulfonamide (B-188);
4-[2-(2-methylpyridin-5-yl)cyclopenten-1-yl]benzenesulfonamide (B-189);
ethyl 2-[4-(4-fluorophenyl)-5-[4-(methylsulfonyl) phenyl]oxazol-2-yl]-2-benzyl-acetate (B-190);
2-[4-(4-fluorophenyl)-5-[4-(methylsulfonyl)phenyl]oxazol-2-yl]acetic acid (B-191);
2-(tert-butyl)-4-(4-fluorophenyl)-5-[4-(methylsulfonyl)phenyl]oxazole (B-192);
4-(4-fluorophenyl)-5-[4-(methylsulfonyl)phenyl]-2-phenyloxazole (B-193);
4-(4-fluorophenyl)-2-methyl-5-[4-(methylsulfonyl)phenyl]oxazole (B-194);
4-[5-(3-fluoro-4-methoxyphenyl)-2-trifluoromethyl-4-oxazolyl]benzenesulfonamide (B-195);
6-chloro-7(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H)-furanone (B-198);
6-chloro-2-trifluoromethyl-2H-1-benzothiopyran-3-carboxylic acid (B-199);
4-[5-(4-chlorophenyl)-3-(trifluoromethyl)-1H-pyrazol-1-yl]benzenesulfonamide (B-200);
4-[5-(4-methylphenyl)-3-(trifluoromethyl)-1H-pyrazol-1-yl]benzenesulfonamide (B-201);
4-[5-(3-fluoro-4-methoxyphenyl)-3-(difluoromethyl)-1H-pyrazol-1-yl]benzenesulfonamide (B-202);
3-[1-[4-(methylsulfonyl)phenyl]-4-trifluoromethyl-1H-imidazol-2-yl]pyridine (B-203);
2-methyl-5-[1-[4-(methylsulfonyl)phenyl]-4-trifluoromethyl-1H-imidazol-2-yl]pyridine (B-204);
4-[2-(5-methylpyridin-3-yl)-4-(trifluoromethyl)-1H-imidazol-1-yl]benzenesulfonamide (B-205);
4-[5-methyl-3-phenylisoxazol-4-yl]benzenesulfonamide (B-206);
4-[5-hydroxymethyl-3-phenylisoxazol-4-yl]benzenesulfonamide (B-207);
[2-trifluoromethyl-5-(3,4-difluorophenyl)-4-oxazolyl]benzenesulfonamide (B-208);
4-[2-methyl-4-phenyl-5-oxazolyl]benzenesulfonamide (B-209);
4-[5-(2-fluoro-4-methoxyphenyl)-2-trifluoromethyl-4-oxazolyl]benzenesulfonamide (B-210);
[2-(2-chloro-6-fluoro-phenylamino)-5-methyl-phenyl]-acetic acid or COX 189 (B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, soldium salt or L-745337 (B-214);
N-[5-(4-fluoro-phenylsulfanyl)-thiophen-2-yl]-methanesulfonamide or RWJ-63556 (B-215);
3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);
(5Z)-2-amino-5-[[3,5-bis(1,1-dimethylethyl)-4-hydroxyphenyl]methylene]-4(5H)-thiazolone or darbufelone (B-217);
CS-502 (B-218);
LAS-34475 (B-219);
LAS-34555 (B-220);
S-33516 (B-221);
SD-8381 (B-222);
L-783003 (B-223);
N-[3-(formylamino)-4-oxo-6-phenoxy-4H-1-benzopyran-7-yl]-methanesulfonamide or T-614 (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);
CGP-28238 (B-228);
4-[[3,5-bis(1,1-dimethylethyl)-4-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231); or
S-2474 (B-232);
or an isomer, a pharmaceutically acceptable salt, ester or prodrug thereof, respectively.

In a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III): 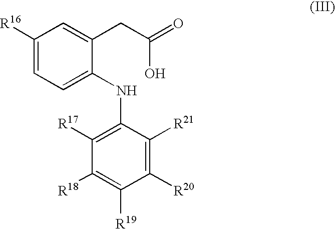
or an isomer, a pharmaceutically acceptable salt, ester, or prodrug thereof;

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:

R¹⁶ is ethyl;

R¹⁷ and R¹⁹ are chloro;

R¹⁸ and R²⁰ are hydrogen; and

and R²¹ is methyl.

According to another embodiment, the invention is directed to a method for the treatment of neoplasia disorders comprising administering to a subject in need thereof, a cyclooxygenase-2 (COX-2) inhibitor in a first amount and an indolinone in a second amount, wherein said first amount together with said second amount is a therapeutically effective amount of said COX-2 inhibitor and an indolinone, and wherein said COX-2 inhibitor is represented by Formula (IV): 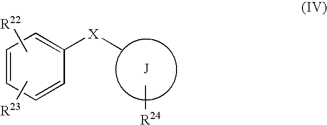
or an isomer, a pharmaceutically acceptable salt, an ester, or a prodrug thereof, wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

An evaluation of the antiinflammatory activity of the cyclooxygenase-2 selective inhibitor, RWJ 63556, in a canine model of inflammation, was described by Kirchner et al., in J Pharmacol Exp Ther 282, 1094-1101 (1997).

According to another embodiment, the COX-2 inhibitors used in combination with an indolinone have the structural Formula (V): 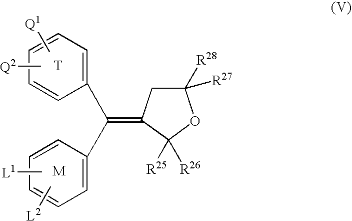
or an isomer, a pharmaceutically acceptable salt, an ester, or a prodrug thereof,

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms, and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Preferred cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

The cyclooxygenase-2 selective inhibitors described above may be referred to herein collectively as COX-2 selective inhibitors, or cyclooxygenase-2 selective inhibitors.

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of the disorder, while avoiding adverse side effects typically associated with alternative therapies. The phrase “therapeutically-effective” is to be understood to be equivalent to the phrase “effective for the treatment or prevention”, and both are intended to qualify the amount of each agent for use in the combination therapy which will achieve the goal of improvement in the severity of neoplasia and the frequency of incidence over treatment of each agent by itself, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

For 3-heteroaryl-2-indolinone compounds used in the methods of the invention, the therapeutically effective dose contained in any combination can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the PTK activity). Such information can be used to more accurately determine useful doses in humans.

Toxicity and therapeutic efficacy of the 3-heteroaryl-2-indolinone compounds contained in any combination described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀.

Indolinone compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase using the assays described herein. Dosages necessary to achieve the MEC will depend on individual characteristics and route of administration. However, HPLC assays or bioassays can be used to determine plasma concentrations.

Dosage intervals can also be determined using MEC value. 3-heteroaryl-2-indolinone compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

In the present method, the amount of a 3-heteroaryl-2-indolinone compound that is used is such that, when administered with the cyclooxygenase-2 selective inhibitor, it is sufficient to constitute an effective amount of the combination. It is preferred that the dosage of the combination constitutes a therapeutically effective amount.

It is preferred that the amount of a 3-heteroaryl-2-indolinone compound that is used in combination with a COX-2 selective inhibitor for a single dosage of treatment is within a range of from about 0.001 mg/kg of body weight of the subject to about 200 mg/kg. It is more preferred that the amount is from about 0.01 mg/kg to about 20 mg/kg, even more preferred that it is from about 0.1 mg/kg to about 12 mg/kg, and yet more preferred that it is from about 0.2 mg/kg to about 10 mg/kg.

The frequency of dose will depend in part upon the half-life of a 3-heteroaryl-2-indolinone compound. If a 3-heteroaryl-2-indolinone compound has a short half life (e.g. from about 2 to 10 hours) it may be necessary to give one or more doses per day. Alternatively, if a 3-heteroaryl-2-indolinone compound has a long half-life (e.g. from about 2 to about 15 days) it may only be necessary to give a dosage once per day, per week, or even once every 1 or 2 months. A preferred dosage rate is to administer the dosage amounts described above to a subject once per day.

Similarly, the amount of COX-2 selective inhibitor that is used in the subject method may be an amount that, when administered with a 3-heteroaryl-2-indolinone compound, is sufficient to constitute an effective amount of the combination. Preferably, such amount would be sufficient to provide a therapeutically effective amount of the combination. The therapeutically effective amount can also be described herein as a neoplasia treatment or prevention effective amount of the combination.

In the present method, the amount of COX-2 selective inhibitor that is used in the novel method of treatment preferably ranges from about 0.01 to about 100 milligrams per day per kilogram of body weight of the subject (mg/day·kg), more preferably from about 0.1 to about 50 mg/day·kg, even more preferably from about 1 to about 20 mg/day·kg.

When the COX-2 selective inhibitor comprises rofecoxib, it is preferred that the amount used is within a range of from about 0.15 to about 1.0 mg/day·kg, and even more preferably from about 0.18 to about 0.4 mg/day˜kg.

When the COX-2 selective inhibitor comprises etoricoxib, it is preferred that the amount used is within a range of from about 0.5 to about 5 mg/day·kg, and even more preferably from about 0.8 to about 4 mg/day·kg.

When the COX-2 selective inhibitor comprises celecoxib, it is preferred that the amount used is within a range of from about 1 to about 10 mg/day·kg, even more preferably from about 1.4 to about 8.6 mg/day·kg, and yet more preferably from about 2 to about 3 mg/day·kg.

In the present method, and in the subject compositions, a 3-heteroaryl-2-indolinone compound is administered with, or is combined with, a COX-2 selective inhibitor. It is preferred that the weight ratio of the amount of a 3-heteroaryl-2-indolinone compound to the amount of COX-2 selective inhibitor that is administered to the subject is within a range of from about 0.0001:1 to about 2000:1, more preferred is a range of from about 0.002:1 to about 1200:1, even more preferred is a range of from about 0.01:1 to about 1:1.

The combination of a 3-heteroaryl-2-indolinone compound and a COX-2 selective inhibitor can be supplied in the form of a novel therapeutic composition that is believed to be within the scope of the present invention. The relative amounts of each component in the therapeutic composition may be varied and may be as described just above. A 3-heteroaryl-2-indolinone compound and COX-2 selective inhibitor that are described above can be provided in the therapeutic composition so that the preferred amounts of each of the components are supplied by a single dosage, a single injection or a single capsule for example, or, by up to four, or more, single dosage forms.

When the novel combination is supplied along with a pharmaceutically acceptable carrier or excipient, a pharmaceutical composition is formed. A pharmaceutical composition of the present invention is directed to a composition suitable for the prevention or treatment of a disease related to tyrosine kinase signal transduction. The pharmaceutical composition comprises a pharmaceutically acceptable carrier, a 3-heteroaryl-2-indolinone compound, and a cyclooxygenase-2 selective inhibitor. In one preferred embodiment, the 3-heteroaryl-2-indolinone compound is 3-[(2,4-Dimethylpyrrol-5-yl)methylene]-2-indolinone (SU5416).

Pharmaceutically acceptable excipients include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a particular pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of cyclooxygenase-2 selective inhibitors. Illustrative pharmaceutically-acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of neoplasia. Besides being useful for human treatment, these combinations are also useful for treatment of mammals, including horses, dogs, cats, rats, mice, sheep, pigs, etc.

The term “subject” for purposes of treatment includes any human or animal subject who is in need of a partcular treatment, especially the prevention of neoplasia or is afflicted with such disorder. The subject is typically a mammal. “Mammal”, as that term is used herein, refers to any animal classified as a mammal, including humans, domestic and farm animals, and zoo, sports, or pet animals, such as dogs, horses, cats, cattle, etc. Preferably, the mammal is a human.

For methods of prevention, the subject is any human or animal subject, and preferably is a subject that is in need of prevention and/or treatment of neoplasia. The subject may be a human subject who is at risk for a disorder or condition, such as neoplasia. The subject may be at risk due to genetic predisposition, sedentary lifestyle, diet, exposure to disorder-causing agents, exposure to pathogenic agents and the like.

The pharmaceutical compositions of the present invention may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “combination therapy”, “co-administration”, “administration with”, or “co-therapy”, in defining the use of a cyclooxygenase-2 inhibitor agent and an indolinone, are intended to embrace administration of each agent in a sequential manner in a regimen that will provide beneficial effects of the drug combination, and are intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

Although the combination of the present invention may include administration of the 3-heteroaryl-2-indolinone component and a cyclooxygenase-2 selective inhibitor component within an effective time of each respective component, it is preferable to administer both respective components contemporaneously, and more preferable to administer both respective components in a single delivery dose.

In particular, the combinations of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The present combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The present invention further comprises kits that are suitable for use in performing the methods of treatment or prevention of neoplasia as described above. In one embodiment, the kit contains a first dosage form comprising a 3-heteroaryl-2-indolinone or related compound and a second dosage form comprising one or more of the cyclooxygenase-2 selective inhibitors or prodrugs thereof, in quantities sufficient to carry out the methods of the present invention. Preferably, the first dosage form and the second dosage form together comprise a therapeutically effective amount of the compounds for the treatment or prevention of neoplasia.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

### EXAMPLES

### Example 1

General Synthesis:

### Method A

A reaction mixture of the proper oxindole (2-indolinone) (1 equiv.), the appropriate aldehyde (1.2 equiv.), and piperidine (0.1 equiv.) in ethanol (1-2 mL/1 mmol oxindole) was stirred at 90° C. for 3-5 h. After cooling, the precipitate was filtered, washed with cold ethanol, and dried to yield the target compound.

### Method B

Preparation of The Proper Aldehydes via Vilsmeier Reaction. To a solution of N,N-dimethylformamide (1.2 equiv.) in 1,2-dichloroethane (2.0 mL /1.0 mmole of starting material) was added dropwise phosphorus oxychloride (1.2 equiv.) at 0° C. The ice-bath was removed and the reaction mixture was further stirred for 30 min. The proper starting material (1.0 equiv.) was added to the above solution portionwise and the reaction mixture was stirred at 50° -70° C. for 5 h-2 days. The reaction mixture was poured into ice-cold 1N sodium hydroxide solution (pH=9 after mixing) and the resulting mixture was stirred at room temperature for 1 h. The organic layer was separated and the aqueous layer was extracted with ethyl acetate. The combined organic layer was washed with brine until pH=7, dried over anhydrous sodium sulfate and evaporated. The residue was chromatographed on a silica gel column eluting with a solvent mixture of ethyl acetate and hexane to afford the title compound.

Synthesis for 3-Substituted-2-Indolinone Analogs. A reaction mixture of the proper oxindole (2-indolinone) (1 equiv.), the appropriate aldehyde (1.2 equiv.), and piperidine (0.1 equiv.) in ethanol (1-2 mL 1 mmol oxindole) was stirred at 90° C. for 3-5 h. After cooling, the precipitate was filtered, washed with cold ethanol and dried to yield the target compound.

### Synthesis Of 3-Benzylidene-2-Indolinone (SU4928)

The preferred method for synthesizing 3-benzylidene-2-indolinone is as follows: Added 123.2 μl of benzaldehyde and 40 μl of piperidine to a solution of 137.0 mg of oxindole in 2.0 ml methanol. Reflux the reaction mixtured for 3 hours and cool down the mixture in an ice-water bath. Filter the resulting precipitate, wash with cold methanol and dry in an oven at 40° C. overnight. Approximately 129.0 mg of the compound was obtained using such protocol.

### Synthesis Of 3-[(Pyrid-4-yl) methylene]-2-indolinone (SU5212)

The preferred method for synthesizing 3-[(Pyrid-4-yl)methylene]-2-indolinone is as follows: Add 117.0 μl of 4-pyridinecarboxaldehyde and 40 μl of piperidine to a solution of 138.0 mg of oxindole in 2.0 ml methanol. The reaction mixture was refluxed for 3 hours and cooled down in an ice-water bath. The resulting precipitate was filtered, washed with cold methanol and dried in an oven at 40° C. overnight to give 134.5 mg of the compound.

### Synthesis of 3-[4-(morpholin-4-yl)benzylidenyl]-2-indolinone (SU4981) (Method B)

4-(Morpholin-4-yl)benzaldehyde. To a solution of 15 mL of N,N-dimethylformamide in 50 mL of 1,2-dichloroethane was added dropwise 10 mL of phosphorus oxychloride at 0° C. The ice-bath was removed and the reaction mixture was further stirred for 30 min. 4-Phenylmorpholine (16.3 g) was added to the above solution portionwise and the reaction mixture was refluxed for 2 days. Triethylamine (2.5 mL) was added to the above reaction mixture and the reaction was refluxed for 2 days. The reaction mixture was poured into ice-cold 1N sodium hydroxide solution (pH=9 after mixing) and the resulting mixture was stirred at room temperature for 1 h. The organic layer was separated and the aqueous layer was extracted with 2×20 mL of dichloromethane. The combined organic layer was washed with brine until pH=7, dried over anhydrous sodium sulfate and evaporated. The residue was separated on a silica gel column eluting with a solvent mixture of ethyl acetate and hexane to afford 12.95 g (68%) of the title compound as a white solid.

### 3-[4-(Morpholin-4-yl)benzylidenyl]-2-indolinone (SU4981)

A reaction mixture of 6.66 g of oxindole, 11.50 g of the 4-(morpholine-4-yl)benzaldehyde, and 5 mL of piperidine in 50 mL of ethanol was stirred at 90° C. for 5 h. After cooling, the precipitate was filtered, washed with cold ethanol, and dried to yield 15.0 g (98%) of the title compound as a yellow solid.

### Synthesis of 3-[4-(4-Formylpiperazin-yl)benzylidenyl)-2-indolinone (SU4984) (Method B)

4-(4-Formylpiperazin-1-yl)benzaldehyde. To a solution of 3.9 mL (30 mmoles) of N,N-dimethylformamide in 20 mL of 1,2-dichloroethane was added dropwise 3.0 mL (3.9 mmoles) of phosphorus oxychloride at 0° C. The ice-bath was removed and the reaction mixture was further stirred for 15 min. 1-Phenylpiperazine (16.0 g, 10 mmoles) was added to the a solution portionwise and the reaction mixture was stirred at 50° C. for 1 h. The reaction mixture was poured into ice-cold 1N sodium hydroxide solution and stirred at room temperature for 1 h. The organic layer was separated and the aqueous layer was extracted with 2.times.20 mL of ethyl acetate. The combined organic layer was washed with brine until pH=7, dried over anhydrous sodium sulfate and evaporated. The residue was separated on a silica gel column eluting with a mixture of ethyl acetate and hexane to afford 9.0 g (41 %) of the title compound a light yellow solid.

### 3-[4-(4-Formylpiperazin-1-yl)benzylidenyl]-2-indolinone (SU4984)

A reaction mixture of 133.15 mg of oxindole, 228.3 mg of 4-(piperazinlyl)benzaldehyde, and 3 drops of piperidine in 2 mL of ethanol was stirred at 90° C. for 5 h. After cooling, the precipitate was filtered, washed with cold ethanol and dried to yield 199.5 mg (65%) of the title compound a yellow solid.

### Synthesis of 3-[4-(Piperidin-1-yl)benzylidenyl]-2-indolinone (SU5450) (Method B)

4-(Piperidin-1-yl)benzaldehyde. To a solution of 2.3 mL (mmoles) of N,N-dimethylformamide in 10 mL of 1,2-dichloroethane was added dropwise 2.8 mL (30 mmoles) of phosphorus oxychloride at 0° C. The ice-bath was removed and the reaction mixture was stirred for 15 min. 1-Phenylpiperidine (3.2 mL, 20 mmoles) was added to the above solution portionwise and the reaction mixture was refluxed overnight. The reaction mixture was poured into ice-cold 2N sodium hydroxide solution and stirred at room temperature for 1 h. The organic layer was separated and the aqueous layer was extracted with 2×20 mL of ethyl acetate. The combined organic layer was washed with brine until pH=7, dried over anhydrous sodium sulfate and evaporated. The residue was separated on a silica gel column eluting with ethyl acetate and hexane to afford 1.5 g (40%) of the title compound as a white solid.

### 3-[4-(Piperidin-1-yl)benzylidenyl]-2-indolinone (SU5450)

A reaction mixture of 134.0 mg of oxindole, 226.8 g of 4-(piperidine-1-yl)benzaldehyde, and 3 drops of piperidine in 2 mL of ethanol was stirred at 90° C. for 5 h. After cooling, the precipitate was filtered, washed with cold ethanol, and dried to yield 268.5 mg (88%) of the title compound as a yellow solid.

### Synthesis of 3-[2-Chloro-4-methoxybenzylidenyl]-2-indolinone (SU5480)

2-Chloro-4-methoxybenzaldehyde. The reaction mixture of 1.0 g (6.4 mmoles) of 2-chloro-4-hydroxybenzaldehyde, 4.4 g (32 mmoles) of potassium carbonate, and 1.4 g (9.6 mmoles) of methyl iodide in 10 mL of N,N-dimethylformamide was stirred at 70° C. for 2 h and poured into ice water. The precipitate was filtered, washed with water, and dried at 40° C. in vacuum oven overnight to yield 750 mg (68%) of the title compound as a light pink solid.

### 3-[2-Chloro-4-methoxybenzylidenyl]-2-indolinone (SU5480)

The reaction mixture of 487.9 mg (3.7 mmoles) of oxindole, 750 mg (4.3 mmoles) of 2-chloro-4-methoxybenzaldehyde and 4 drops of piperidine in 5 mL of ethanol was heated to 90° C. for 2 h and cooled to room temperature. The yellow precipitate was filtered, washed with cold ethanol, and dried at 40° C. in a vacuum oven overnight to give 680.2 mg (62%) of the title compound.

### Synthesis of 3-[(4-Methylthien-2-yl)methylene]-2-indolinone (SU5401)

A reaction mixture of 133.0 mg of oxindole, 151.2 mg of the 4-methylthiophene-2-carboxaldehyde, and 3 drops of piperidine in 3 mL of ethanol was stirred at 90° C. for 3 h. After cooling, the precipitate was filtered, washed with cold ethanol, and dried to yield 147.3 mg (61%) of the title compound as a yellow solid.

### Synthesis of 3-[(3-Methylpyrrol-2-yl)methylene]-2-indolinone (SU5404)

A reaction mixture of 133.0 mg of oxindole, 130.9 mg of the 3-methylpyrrole-2-carboxaldehyde, and 3 drops of piperidine in 2 mL of ethanol was stirred at 90° C. for 3 h. After cooling, the precipitate was filtered, washed with cold ethanol, and dried to yield 150.9 mg (67%) of the title compound as a yellow solid.

### Synthesis of 3-[(3,4-Dimethylpyrrol-2-yl)methylene]-2-indolinone (SU5406)

3-[(3,4-Dimethylpyrrol-2-yl)methylene]-2-indolinone was synthesized as described in J. Heterocyclic Chem. 13:1145-1147 (1976).

Ethyl 4-methylpyrrol-3-carboxylate. A solution of 11.86 g (0.1 moles) of ethyl crotonate and 19.50 g (0.1 moles) of p-toluenesulfonylmethylisocyanide in 500 mL of a 2:1 ether/dimethylsulfoxide was added dropwise into a suspension of 6.8 g of sodium hydride (60% mineral oil dispension, 0.17 moles) in ether at room temperature. Upon completion of addition the reaction mixture was stirred for 30 min and diluted with 400 mL of water. The aqueous layer was extracted with 3×100 mL of ether. The combined ether extracts were passed through a column of alumina eluting with dichloromethane. The organic solvent was evaporated and the resulting residue was solidified on standing. The solid was washed with hexane and dried at 40° C. in vacuum oven overnight to yield 12.38 g (80%) of the title compound.

Preparation of 3,4-Dimethylpyrrole. To a solution of 23 g (80 mmoles) of sodium dihydrobis(2-methoxyethoxy aluminate) was added dropwise of a solution of 5 g (34 mmoles) of ethyl 4-methylpyrrol-3-carboxylate in 50 mL of benzene at room temperature under nitrogen atmosphere. The reaction mixture was stirred for 18 h. Water (100 mL) was added to the reaction mixture. The organic layer was separated, washed with brine and dried over anhydrous sodium sulfate. The solvent was removed and the residue was distilled giving 1.2 g (44%) of the title compound.

Preparation of 3,4-Dimethylpyrrole-2-carboxaldehyde. To a solution of 0.92 mL (12 mmoles) of N,N-dimethylformamide in mL of 1,2-dichloroethane was added dropwise 1.0 mL (12 mmoles) of phosphorus oxychloride at 0° C. The ice-bath was removed and the reaction mixture was further stirred for 30 min. 3,4-Dimethylpyrrole (960.0 mg, 10 mmoles) was added to the above solution portionwise and the reaction mixture was stirred at 50° C. for 5 h. The reaction mixture was poured into ice-cold 1N sodium hydroxide solution (pH=9 after mixing) and the resulting mixture was stirred at room temperature for 1 h. The organic layer was separated and the aqueous layer was extracted with ethyl acetate. The combined organic layer was washed with brine until pH=7, dried over anhydrous sodium sulfate and evaporated. The residue was chromatographed on a silica gel column eluting with a solvent mixture of ethyl acetate and hexane to afford 610 mg (50%) of the title compound.

### 3-[(3,4-Dimethylpyrrol-2-yl)methylene]-2-indolinone (SU5406)

A reaction mixture of 67.0 mg (0.5 mmoles) of oxindole, 73.0 mg (0.6 mmoles) of the 3,4-dimethylpyrrole-2-carboxaldehyde, and 2 drops of piperidine in 2 was stirred at 90° C. for 3 h. After cooling, the precipitate was filtered, washed with cold ethanol, and dried to yield 87.7 mg (37%) of the title compound as a yellow solid.

### Synthesis of 3-[(2,4-Dimethyl-3-ethoxycarbonylpyrrol-5-yl)methylene]-2-indolinone (SU5408)

A reaction mixture of 134.0 mg of oxindole, 234.3 mg of the 4-ethoxycarbonyl-3,5-dimethylpyrrole-2-carboxaldehyde, and 3 drops of piperidine in 3 mL of ethanol was stirred at 90° C. for 3 h. After cooling, the precipitate was filtered, washed with cold ethanol, and dried to yield 244.6 mg (79%) of the title compound as a yellow solid.

### Synthesis of 3-[(2,4-Dimethylpyrrol-5-yl)methylene]-2-indolinone (SU5416)

A reaction mixture of 134.0 mg of oxindole, 147.8 mg of the 3,5-dimethylpyrrole-2-carboxaldehyde, and 3 drops of piperidine in 2 mL of ethanol was stirred at 90° C. for 3 h. After cooling, the precipitate was filtered, washed with cold ethanol, and dried to yield 136.7 mg (57%) of the title compound as a yellow solid.

### Synthesis of 3-[(2-Methylmercaptothien-5-yl)methylene]-2-indolinone (SU5419)

A reaction mixture of 134.0 mg of oxindole, 189.9 mg of the 5-methylmercaptothiophene-2-carboxaldehyde, and 3 drops of piperidine in 2 mL of ethanol was stirred at 90° C. for 3 h. After cooling, the precipitate was filtered, washed with cold ethanol, and dried to yield 246.6 mg (90%) of the title compound as a orange solid.

### Synthesis of 3-[(2-Methylthien-5-yl)methylene]-2-indolinone (SU5424)

A reaction mixture of 134.0 mg of oxindole, 151.42 mg of the 5-methylthiophene-2-carboxaldehyde, and 3 drops of piperidine in 2 mL of ethanol was stirred at 90° C. for 3 h. After cooling, the precipitate was filtered, washed with cold ethanol, and dried to yield 237.8 mg (99%) of the title compound as a yellow solid.

### Synthesis of 3-[(3-Methylthien-2-yl)methylene]-2-indolinone (SU5427)

A reaction mixture of 134.0 mg of oxindole, 151.4 mg of the 3-methylthiophene-2-carboxaldehyde, and 3 drops of piperidine in 2 mL of ethanol was stirred at 90° C. for 3 h. After cooling, the precipitate was filtered, washed with cold ethanol, and dried to yield 157.8 mg (65%) of the title compound as a yellow solid.

### Synthesis of 3-(2,5-Dimethoxybenzylidenyl)-2-indolinone (SU4793)

3-(2,5-Dimethoxybenzylidenyl)-2-indolinone is synthesized according to Method A.

### Synthesis of 3-(2,3-dimethoxybenzylidenyl)-2-indolinone (SU4794)

3-(2,3-dimethoxybenzylidenyl)-2-indolinone is ynthesized according to Method A.

### Synthesis of 3-(3-bromo-6-methoxybenzylidenyl)-2-indolinone (SU4796)

3-(3-bromo-6-methoxybenzylidenyl)-2-indolinone is synthesized according to Method A.

### Synthesis of 3-[4-(4-t-butylcarbonyl-piperazin-1-yl)benzylidenyl)-2-indolinone (SU5393)

3-[4-(4-t-butylcarbonyl-piperazin-1-yl)benzylidenyl]-2-ndolinone is synthesized according to Method B.

### Synthesis of 3-[(furan-2-yl)methylene]-2-indolinone (SU4798)

3-[(furan-2-yl)methylene]-2-indolinone is synthesized according to Method A.

### Synthesis of 3-(4-acetamidobenzylidenyl)-2-indolinone (SU4799)

3-(4-acetamidobenzylidenyl)-2-indolinone is synthesized according to Method A.

### Synthesis of 3-(2-chloro-4-hydroxybenzylidenyl)-2-indolinone (SU4932)

3-(2-chloro-4-hydroxybenzylidenyl)-2-indolinone is synthesized according to Method A.

### Synthesis of 3-(4-Bromobenzylidenyl)-2-indolinone (SU4942)

3-(4-Bromobenzylidenyl)-2-indolinone is synthesized according to Method A.

### Synthesis of 3-(4-Acetylaminobenzylidenyl)-2-indolinone (SU4944)

3-(4-Acetylaminobenzylidenyl)-2-indolinone is synthesized according to Method A.

### Synthesis of 3-(2-Methoxybenzylidenyl)-2-indolinone (SU4949)

3-(2-Methoxybenzylidenyl)-2-indolinone is synthesized according to Method A.

### Synthesis of 3-(4-Dimethylaminobenzylidenyl)-1-methyl-2-indolinone (SU4952)

3-(4-Dimethylaminobenzylidenyl)-1-methyl-2-indolinone is synthesized according to Method A.

### Synthesis of 3-(4-Dimethylaminobenzylidenyl)-2-indolinone (SU4312)

3-(4-Dimethylaminobenzylidenyl)-2-indolinone is available from Maybridge Chemical Co. Ltd.

### Synthesis of 3-(4-Bromobenzylidenyl)-1-methyl-2-indolinone (SU4956)

3-(4-Bromobenzylidenyl)-1-methyl-2-indolinone is synthesized according to Method A.

### Synthesis of 5-Chloro-3-(4-dimethylaminobenzylidenyl)-2-indolinone (SU4967)

5-Chloro-3-(4-dimethylaminobenzylidenyl)-2-indolinone is synthesized according to Method A.

### Synthesis of 3-(4-Bromobenzylidenyl)-5-chloro-2-indolinone (SU4972)

3-(4-Bromobenzylidenyl)-5-chloro-2-indolinone is synthesized according to Method A.

### Synthesis of 3-(4-Diethylaminobenzylidenyl)-2-indolinone (SU4978)

3-(4-Diethylaminobenzylidenyl)-2-indolinone is synthesized according to Method A.

### Synthesis of 3-(4-Di-n-butylaminobenzylidenyl)-2-indolinone (SU4979)

3-(4-Di-n-butylaminobenzylidenyl)-2-indolinone is synthesized according to Method A.

### Synthesis of 1-Methyl-3-[4-(morpholin-4-yl)benzylidenyl]-2-indolinone (SU4982)

1-Methyl-3-[4-(morpholin-4-yl)benzylidenyl]-2-indolinone is synthesized according to Method B.

### Synthesis of 5-Chloro-3-(4-(morpholine-4-yl)benzylidenyl]-2-indolinone (SU4983)

5-Chloro-3-(4-(morpholine-4-yl)benzylidenyl]-2-indolinone is synthesized according to Method B.

### Synthesis of 3-(3,4-Dichlorobenzylidenyl)-2-indolinone (SU5201)

3-(3,4-Dichlorobenzylidenyl)-2-indolinone is synthesized according to Method A.

### Synthesis of 3-(2-Ethoxybenzylidenyl]-2-indolinone (SU5204)

3-(2-Ethoxybenzylidenyl]-2-indolinone is synthesized according to Method A.

### Synthesis of 3-(4-Fluorobenzylidenyl)-2-indolinone (SU5205)

3-(4-Fluorobenzylidenyl)-2-indolinone is synthesized according to Method A.

### Synthesis of 3-[(Thien-2-yl)methylene]-2-indolinone (SU5208)

3-[(Thien-2-yl)methylene]-2-indolinone is synthesized according to Method A.

### Synthesis of 3-(2-Methoxybenzylidenyl)-2-indolinone (SU5214)

3-(2-Methoxybenzylidenyl)-2-indolinone is synthesized according to Method A.

### Synthesis of 3-[2-[3,5-Di-(trifluoromethyl)phenyl]furan-5-yl]methylene]-2-indolinone (SU5217)

3-[2-[(3,5-Di-(trifluoromethyl)phenyl]furan-5-yl]methylene]-2-indolinone is synthesized according to Method A.

### Synthesis of 2,6-Di-(dimethylamino)-3,5-di-[(indolin-2-one-3-ylidenyl)met hyl]-phenylcyanide (SU5218)

2,6-Di-(dimethylamino)-3,5-di-[(indolin-2-one-3-ylidenyl)met hyl]-phenylcyanide is synthesized according to Method A.

### Synthesis of 3-[(3-(2-carboxyethyl)-4-methylpyrrol-5-yl)methylene]-2-indo linone (SU5402)

3-[(3-(2-carboxyethyl)4-methylpyrrol-5-yl)methylene]-2-indo linone is synthesized according to Method

### Synthesis of 3-[(3,4-Dibromo-5-methylpyrrol-2-yl)methylene]-2-indolinone (SU5403)

3-[(3,4-Dibromo-5-methylpyrrol-2-yl)methylene]-2-indolinone is synthesized according to Method B.

### Synthesis of 3-[(3,4-Dimethyl-2-formylpyrrole-5-yl)methylene)-2-indolinone (SU5405)

3-[(3,4-Dimethyl-2-formylpyrrole-5-yl)methylene)-2-indolinone is synthesized according to Method A.

### Synthesis of 3-{[4-(2-methoxycarbonylethyl)-3-methylpyrrol-5-yl]methylene}-2-indolin (SU5407)

3-{[4-(2-methoxycarbonylethyl)-3-methylpyrrol-5-yl]methylene}-2-indolinone is synthesized accord Method A.

### Synthesis of 3-[2-Iodofuran-5-yl)methylene]-2-indolinone (SU5409)

3-[2-Iodofuran-5-yl)methylene]-2-indolinone is synthesized according to Method A.

### Synthesis of 3-[(3-Ethoxycarbonyl-2-methylfuran-5-yl)methylene]-2-indolin one (SU5410)

3-[(3-Ethoxycarbonyl-2-methylfuran-5-yl)methylene]-2-indolinone is synthesized according to Method A.

### Synthesis of 3-[(3-Bromothiene-2-yl)methylene]-2-indolinone (SU5418)

3-[(3-Bromothiene-2-yl)methylene]-2-indolinone is ynthesized according to Method A.

### Synthesis of 3-[(2-Chlorothiene-5-yl)methylene)-2-indolinone (SU5420)

3-[(2-Chlorothiene-5-yl)methylene)-2-indolinone is ynthesized according to Method A.

### Synthesis of 3-[(2,3-Dimethylfuran-5-yl)methylene]-2-indolinone (SU5421)

3-[(2,3-Dimethylfuran-5-yl)methylene]-2-indolinone is synthesized according to Method A.

### Synthesis of 3-[(5-Nitrothien-2-yl)methylene]-5 2-indolinone (SU5422)

3-[(5-Nitrothien-2-yl)methylene]-2-indolinone is synthesized according to Method A.

### Synthesis of 3-[(2-Carboxythien-5-yl)methylene]-2-indolinone (SU5423)

3-[(2-Carboxythien-5-yl)methylene]-2-indolinone is synthesized according to Method A.

### Synthesis of 3-[(2-Bromothiene-5-yl)methylene]-2-indolinone (SU5425)

3-[(2-Bromothiene-5-yl)methylene]-2-indolinone is synthesized according to Method A.

### Synthesis of 3-[(4-Bromothiene-2-yl)methylene]-2-indolinone (SU5426)

3-[(4-Bromothiene-2-yl)methylene]-2-indolinone is synthesized according to Method A.

### Synthesis of 3-[(2-Sulphonylfuran-5-yl)methylene]-2-indolinone sodium salt (SU5428)

3-[(2-Sulphonylfuran-5-yl)methylene]-2-indolinone sodium salt is synthesized according to Method A.

### Synthesis of 3-[(Furan-2-yl)methylene]-2-indolinone (SU5429)

3-[(Furan-2-yl)methylene]-2-indolinone is synthesized according to Method A.

### Synthesis of 3-[(2-Methylfuran-5-yl)methylene]-2-indolinone (SU5430)

3-[(2-Methylfuran-5-yl)methylene]-2-indolinone is synthesized according to Method A.

### Synthesis of 3-[(2-Ethylfuran-5-yl)methylene-2-indolinone (SU5431)

3-[(2-Ethylfuran-5-yl)methylene-2-indolinone is synthesized according to Method A.

### Synthesis of 3-[(2-Nitrofuran-5-yl)methylene]-2-indolinone (SU5432)

3-[(2-Nitrofuran-5-yl)methylene]-2-indolinone is synthesized according to Method A.

### Synthesis of 3-[(5-Bromofuran-2-yl)methylene]-2-indolinone (SU5438)

3-[(5-Bromofuran-2-yl)methylene]-2-indolinone is synthesized according to Method A.

### Synthesis of 3-[(2-Ethylthien-5-yl)methylene]-2-indolinone (SU5451)

3-[(2-Ethylthien-5-yl)methylene]-2-indolinone is synthesized according to Method A.

### Synthesis of 3-[(4,5-Dimethyl-3-ethylpyrrol-2-yl)methylene]-2-indolinone (SU5453)

3-[(4,5-Dimethyl-3-ethylpyrrol-2-yl)methylene]-2-indolinone is synthesized according to Method A.

### Synthesis of 3-[(5-Ethoxycarbonyl-4-ethoxycarbonylethyl-3-ethoxycarbonylm ethylpyrrol-2-yl)methylene]-2-indolinone (SU5454)

3-[(5-Ethoxycarbonyl-4-ethoxycarbonylethyl-3-ethoxycarbonylm ethylpyrrol-2-yl)methylene]-2-indolinone is synthesized according to Method A.

### Synthesis of 3-[(5-Carboxy-3-ethyl-4-methylpyrrol-2-yl)methylene]-2-indolinone (SU5455)

3-[(5-Carboxy-3-ethyl-4-methylpyrrol-2-yl)methylene]-2-indolinone is synthesized according to

### Synthesis of 3-[(3,5-Diiodo-4-methylpyrrol-2-yl)methylene]-2-indolinone (SU5456)

3-[(3,5-Diiodo-4-methylpyrrol-2-yl)methylene]-2-indolinone is synthesized according to Method A.

### Synthesis of 3-[(5-Chloro-3-methoxycarbonyl-4-methoxycarbonylmethylpyrrol-2-yl)methylene]-2-indolinone (SU5459)

3-[(5-Chloro-3-methoxycarbonyl-4-methoxycarbonylmethylpyrrol-2-yl)methylene]-2-indolinone is synthesized according to Method A.

### Synthesis of 3-[(3-Acetyl-5-ethoxycarbonyl-4-methylpyrrol)-2-yl)methylene]-2-indolinone (SU5460)

3-[(3-Acetyl-5-ethoxycarbonyl-4-methylpyrrol)-2-yl)methylene]-2-indolinone is synthesized according to Method A.

### Synthesis of 3-{[1-(3,5-Dichlorophenyl)pyrrol-2-yl]methylene}-2-indolinone (SU5461)

3-{[1-(3,5-Dichlorophenyl)pyrrol-2-yl]methylene}-2-indolinone is synthesized according to Method A.

### Synthesis of 3-[1-(4-Chlorophenyl)pyrrol-2-yl)methylene]-2-indolinone (SU5462)

3-[1-(4-Chlorophenyl)pyrrol-2-yl)methylene]-2-indolinone is synthesized according to Method A.

### Synthesis of 3-[(4-Ethoxycarbonyl-3-methyl)pyrrol-2-yl)methylene]-2-indolinone (SU5463)

3-[(4-Ethoxycarbonyl-3-methyl)pyrrol-2-yl)methylene]-2-ndolinone is synthesized according to Method A.

### Synthesis of 3-[(1-Methylpyrrol-2-yl)methylenej-2-indolinone (SU5464)

3-[(1-Methylpyrrol-2-yl)methylene-2-indolinone is synthesized according to Method A.

### Synthesis of 3-[(5-Ethoxycarbonyl-3-ethoxycarbonylethyl-4-ethoxylcarbonyl methylpyrrol-2-yl)methylene]-2-indolinone (SU5465)

3-[(5-Ethoxycarbonyl-3-ethoxycarbonylethyl-4-ethoxylcarbonyl methylpyrrol-2-yl)methylene]-2-is synthesized according to Method A.

### Synthesis of 3-[4-(Pyrrolidin-1-yl)benzylidenyl]-2-indolinone (SU5466)

3-[4-(Pyrrolidin-1-yl)benzylidenyl]-2-indolinone is synthesized according to Method A.

### Synthesis of 3-[(5-Methylimidazol-2-yl)methylene]-2-indolinone (SU5468)

3-[(5-Methylimidazol-2-yl)methylene]-2-indolinone is synthesized according to Method A.

### Synthesis of 3-[(5-Methylthiazol-2-yl)methylene]-2-indolinone (SU5469)

3-[(5-Methylthiazol-2-yl)methylene]-2-indolinone is synthesized according to Method A.

### Synthesis of 3-[(3-Methylpyrazol-5-yl)methylene]-2-indolinone (SU5472)

3-[(3-Methylpyrazol-5-yl)methylene]-2-indolinone is synthesized according to Method A.

### Synthesis of 3-[(Imidazol-4-yl)methylene]-2-indolinone (SU5473)

3-[(Imidazol-4-yl)methylene]-2-indolinone is synthesized according to Method A.

### Synthesis of 3-[(4-Chloropyrazol-3-yl)methylene]-2-indolinone (SU5474)

3-[(4-Chloropyrazol-3-yl)methylene]-2-indolinone is synthesized according to Method A.

### Synthesis of 3-[(4-Bromo-1-(4-chlorobenzyl)pyrazol-5-yl)methylene]-2-indolinone (SU5475)

3-[(4-Bromo-1-(4-chlorobenzyl)pyrazol-5-yl)methylene]-2-indolinone is synthesized according to Method A.

### Synthesis of 3-[(4-Chloro-1-methylpyrazol-3-yl)methylene]-2-indolinone (SU5476)

3-[(4-Chloro-1-methylpyrazol-3-yl)methylene]-2-indolinone is synthesized according to Method A.

### Synthesis of 3-[(4-Ethyl-3,5-dimethylpyrrol-2-yl)methylene]-2-indolinone (SU5477)

3-[(4-Ethyl-3,5-dimethylpyrrol-2-yl)methylene]-2-indolinone is synthesized according to Method B.

### Synthesis of 3-[(5-Ethylpyrrol-2-yl)methylene]-2-indolinone (SU5478)

3-[(5-Ethylpyrrol-2-yl)methylene]-2-indolinone is synthesized according to Method B.

### Synthesis of 3-E3,5-Dimethyl-4-(propen-2-yl)pyrrol-2-yl)methylene]-2-indolinone (SU5479)

3-[3,5-Dimethyl-4-(propen-2-yl)pyrrol-2-yl)methylene]-2-indolinone is synthesized according to Method B.

### Synthesis of 5,6-Dimethoxyl-3-[2,3-dimethoxylbenzylidenyl]-2-indolinone (SU5495)

5,6-Dimethoxyl-3-[2,3-dimethoxylbenzylidenyl]-2-indolinone is synthesized according to Method A.

### Synthesis of 3-[2,4,6-Trimethoxybenzylidenyl]-2-indolinone (SU5607)

3-[2,4,6-Trimethoxybenzylidenyl]-2-indolinone is synthesized according to Method A.

### Synthesis of 5-Chloro-3-[(pyrrol-2-yl)methylene]-2-indolinone (SU5612)

5-Chloro-3-[(pyrrol-2-yl)methylene]-2-indolinone is synthesized according to Method A.

### Synthesis of 5-Chloro-3-[(3-methylpyrrol-2-yl)methylene]-2-indolinone (SU5613)

5-Chloro-3-[(3-methylpyrrol-2-yl)methylene]-2-indolinone is synthesized according to Method A.

### Synthesis of 3-(4-isopropylbenzylidenyl)-2-indolinone (SU4313)

3-(4-isopropylbenzylidenyl)-2-indolinone is available from Maybridge Chemical Co. Ltd.

### Synthesis of 5-Chloro-3-[(3,5-dimethylpyrrol-2-yl)methylene]-2-indolinone (SU5614)

5-Chloro-3-[(3,5-dimethylpyrrol-2-yl)methylene]-2-indolinone is synthesized according to Method A.

### Synthesis of 3-[(pyrrol-2-yl)methylene]-2-indolinone (SU4314)

3-[(pyrrol-2-yl)methylene]-2-indolinone is available from Maybridge Chemical Co. Ltd.

### Synthesis of 5-Chloro-3-[(indol-3-yl)methylene]-2-indolinone (SU5615)

5-Chloro-3-[(indol-3-yl)methylene]-2-indolinone is synthesized according to Method A.

### Synthesis of 5-Chloro-3-[(thien-2-yl)methylene]-2-indolinone (SU5616)

5-Chloro-3-[(thien-2-yl)methylene]-2-indolinone is synthesized according to Method A.

### Synthesis of 5-Chloro-3-[(3-methylthien-2-yl)methylene]-2-indolinone-(SU5617)

5-Chloro-3-[(3-methylthien-2-yl)methylene]-2-35 indolinone is synthesized according to Method A.

### Synthesis of 5-Chloro-3-[(5-methylthien-2-yl)methylene]-2-indolinone (SU5618)

5-Chloro-3-[(5-methylthien-2-yl)methylene]-2-indolinone is synthesized according to Method A.

### Synthesis of 5-Chloro-3-[(5-ethylthien-2-yl)methylene]2-indolinone (SU5619)

5-Chloro-3-[(5-ethylthien-2-yl)methylene]-2-indolinone is synthesized according to Method A.

### Synthesis of 5-Chloro-3-[(5-methylmercaptothien-2-yl)methylene]-2-indolinone (SU5620)

5-Chloro-3-[(5-methylmercaptothien-2-yl)methylene]-indolinone is synthesized according to Method A.

### Synthesis of 5-Chloro-3-[(imidazol-2-yl)methylene]-2-indolinone (SU5621)

5-Chloro-3-[(imidazol-2-yl)methylene]-2-indolinone is synthesized according to Method A.

### Synthesis of 3-[2,4-Dimethoxy-6-methylbenzylidenyl]2-indolinone (SU5623)

3-[2,4-Dimethoxy-6-methylbenzylidenyl]-2-indolinone synthesized according to Method A.

### Synthesis of 5-Nitro-3-[(pyrrol-2-yl)methylene]-2-indolinone (SU5624)

5-Nitro-3-[(pyrrol-2-yl)methylene]-2-indolinone is nthesized according to Method A.

### Synthesis of 3-[(3-Methylpyrrol-2-yl)methylene]-5-nitro-2-indolinone (SU5625)

3-[(3-Methylpyrrol-2-yl)methylene]-5-nitro-2-olinone is synthesized according to Method A.

### Synthesis of 3-[(3,5-Dimethylpyrrol-2-yl)methylene]5-nitro-2-indolinone (SU5626)

3-[(3,5-Dimethylpyrrol-2-yl)methylene]-5-nitro-2-indolinone is synthesized according to Method A.

### Synthesis of 3-[(Indol-3-yl)methylene]-5-nitro-2-indolinone (SU5627)

3-[(Indol-3-yl)methylene]-5-nitro-2-indolinone is synthesized according to Method A.

### Synthesis of 5-Nitro-3-[(thien-2-yl)methylene]-2-indolinone (SU5628)

5-Nitro-3-[(thien-2-yl)methylene]-2-indolinone is synthesized according to Method A.

### Synthesis of 3-[(3-Methylthien-2-yl)methylene]-5-nitro-2-indolinone (SU5629)

3-[(3-Methylthien-2-yl)methylene]-5-nitro-2-ndolinone is synthesized according to Method A.

### Synthesis of 3-[(S-Methylthien-2-yl)methylene]-5-nitro-2-indolinone (SU5630)

3-[(5-Methylthien-2-yl)methylene]-5-nitro-2-ndolinone is synthesized according to Method A.

### Synthesis of 3-[(5-Ethylthien-2-yl)methylene]-5-nitro-2-indolinone (SU5631)

3-[(5-Ethylthien-2-yl)methylene]-5-nitro-2-dolinone is synthesized according to Method A.

### Synthesis of 3-[(5-Methylmercaptothien-2-yl)methylene]-5-nitro-2-indolinone (SU5632)

3-[(5-Methylmercaptothien-2-yl)methylene]-5-nitro-2-olinone is synthesized according to Method A.

### Synthesis of 3-[(Imidazol-2-yl)methylene]-5-nitro-2-indolinone (SU5633)

3-[(Imidazol-2-yl)methylene]-5-nitro-2-indolinone is synthesized according to Method A.

### Synthesis of 3-[(Oxazol-2-yl)methylene]-2-5 indolinone (CS7127)

3-[(Oxazol-2-yl)methylene]-2-indolinone is synthesized according to Method A.

### Synthesis of 3-[(Oxazol-4-yl)methylene]-2-indolinone (CS7128)

3-[(Oxazol-4-yl)methylene]-2-indolinone is synthesized according to Method A.

### Synthesis of 3-[(Oxazol-5-yl)methylene]-2-indolinone (CS7129)

3-[(Oxazol-5-yl)methylene]-2-indolinone is synthesized according to Method A.

### Synthesis of 3-[(Thiazol-2-yl)methylene]-2-indolinone (CS7130)

3-[(Thiazol-2-yl)methylene]-2-indolinone is synthesized according to Method A.

### Synthesis of 3-[(Thiazol-4-yl)methylene]-2-indolinone (CS7131)

3-[(Thiazol-4-yl)methylene]-2-indolinone is synthesized according to Method A.

### Synthesis of 3-[(Thiazol-5-yl)methylene]-2-indolinone (CS7132)

3-[(Thiazol-5-yl)methylene]-2-indolinone is synthesized according to Method A.

### Synthesis of 3-[(Imidazol-2-yl)methylene]-2-indolinone (CS7133)

3-[(Imidazol-2-yl)methylene]-2-indolinbne is synthesized according to Method A.

### Synthesis of 3-[(Pyrazol-3-yl)methylene]-2-indolinone (CS7135)

3-[(Pyrazol-3-yl)methylene]-2-indolinone is synthesized according to Method A.

### Synthesis of 3-[(Pyrazol-4-yl)methylene]-2-indolinone (CS7136)

3-[(Pyrazol-4-yl)methylene]-2-indolinone is synthesized according to Method A.

### Synthesis of 3-[(Isoxazol-3-yl)methylene]-2-indolinone (CS7137)

3-[(Isoxazol-3-yl)methylene]-2-indolinone is ynthesized according to Method A.

### Synthesis of 3-[(Isoxazol-4-yl)methylene]-2-indolinone (CS7138)

3-[(Isoxazol-4-yl)methylene]-2-indolinone is ynthesized according to Method A.

### Synthesis of 3-[(Isoxazol-5-yl)methylene]-2-indolinone (CS7139)

3-[(Isoxazol-5-yl)methylene]-2-indolinone is ynthesized according to Method A.

### Synthesis of 3-[(Isothiazol-3-yl)methylene]-2-indolinone (CS7140)

3-[(Isothiazol-3-yl)methylene]-2-indolinone is synthesized according to Method A.

### Synthesis of 3-[(Isothiazol-4-yl)methylene]-2-indolinone (CS7141)

3-[(Isothiazol-4-yl)methylene]-2-indolinone is synthesized according to Method A.

### Synthesis of 3-[(Isothiazol-5-yl)methylene]-2-indolinone (CS7142)

3-[(Isothiazol-5-yl)methylene]-2-indolinone is thesized according to Method A.

### Synthesis of 3-[(1,2,3-Triazol-4-yl)methylene]2-indolinone (CS7143)

3-[(1,2,3-Triazol-4-yl)methylene]-2-indolinone is synthesized according to Method A.

### Synthesis of 3-[(1,3,4-Thiadiazol-2-yl)methylene]-2-indolinone (CS7144)

3-[(1,3,4-Thiadiazol-2-yl)methylene]-2-indolinone is synthesized according to Method A.

### Synthesis of 3-[(5-Phenyl-1,2,4-oxadiazol-3-yl)methylene]-2-indolinone (CS7145)

3-[(5-Phenyl-1,2,4-oxadiazol-3-yl)methylene]-2-indolinone is synthesized according to Method A.

### Synthesis of 3-[(3-Phenyl-1,2,4-oxadiazol-5-yl)methylene]-2-indolinone (CS7146)

3-[(3-Phenyl-1,2,4-oxadiazol-5-yl)methylene]-2-indolinone is synthesized according to Method A.

### Synthesis of 3-[(3-Phenyl-1,2,5-oxadiazol-4-yl)methylene]-2-indolinone (CS7147)

3-[(3-Phenyl-1,2,5-oxadiazol-4-yl)methylene]-2-indolinone is synthesized according to Method A.

### Example 2

In Vitro RTK Assays

The following in vitro assays may be used to determine the level of activity and effect of the different compounds of the present invention on one or more of the RTKs. Similar assays can be designed along the same lines for any tyrosine kinase using techniques well known in the art.

Enzyme Linked Immunosorbent Assay (ELISA)

Enzyme linked immunosorbent assays (ELISA) may be used to detect and measure the presence of tyrosine kinase activity. The ELISA may be conducted according to known protocols which are described in, for example, Voller, et al., 1980, “Enzyme-Linked Immunosorbent Assay,” In: Manual of Clinical Immunology, 2d ed., edited by Rose and Friedman, pp. 359-371 Am. Soc. Of Microbiology, Washington, D.C.

The disclosed protocol may be adapted for determining activity with respect to a specific RTK. For example, the preferred protocols for conducting the ELISA experiments for specific RTKs is provided below. Adaptation of these protocols for determining a compound's activity for other members of the RTK family, as well as non-receptor tyrosine kinases, are within the scope of those in the art.

### FLK-1 ELISA

An ELISA assay was conducted to measure the kinase activity of the FLK-1 receptor and more specifically, the inhibition or activation of protein tyrosine kinase activity on the FLK-1 receptor. Specifically, the following assay was conducted to measure kinase activity of the FLK-1 receptor in FLK-1/NIH3T3 cells.

Materials And Methods.

Materials. The following Reagents and Supplies were Used:

a. Corning 96-well ELISA plates (Corning Catalog No. 25805-96);
b. Cappel goat anti-rabbit IgG (catalog no. 55641);
c. PBS (Gibco Catalog No. 450-1300EB);
d. TBSW Buffer (50 mM Tris (pH 7.2), 150 mM NaCl and 0.1% Tween-20);
e. Ethanolamine stock (10% ethanolamine (pH 7.0), stored at 4° C.);
f. HNTG buffer (20 mM HEPES buffer (pH 7.5), 150 mM NaCl, 0.2% Triton X-100, and 10% glycerol);
g. EDTA (0.5M (pH 7.0) as a 100X stock);
h. Sodium ortho vanadate (0.5M as a 100X stock);
i. Sodium pyro phosphate (0.2M as a 100X stock);
j. NUNC 96 well V bottom polypropylene plates (Applied Scientific Catalog No. AS-72092);
k. NIH3T3 C7#3 Cells (FLK-1 expressing cells);
l. DMEM with 1× high glucose L Glutamine (catalog No. 11965-050);
m. FBS, Gibco (catalog no. 16000-028);
n. L-glutamine, Gibco (catalog no. 25030-016);
o. VEGF, PeproTech, Inc. (catalog no. 100-20)(kept as 1 μg/100 μl stock in Milli-Q dH₂ O and stored at −20° C. Affinity purified anti-FLK-1 antiserum, Enzymology Lab, Sugen, Inc.;
q. UB40 monoclonal antibody specific for phosphotyrosine, Enzymology Lab, Sugen, Inc. (see, Fendly, et al., 1990, Cancer Research 50:1550-1558);
r. EIA grade Goat anti-mouse IgG-POD (BioRad catalog no. 172-1011);
s. 2,2-azino-bis(3-ethylbenz-thiazoline-6-sulfonic acid (ABTS) solution (100 mM citric acid (anhydrous), 250 mM Na₂ HPO₄ (pH 4.0), 0.5 mg/ml ABTS (Sigma catalog no. A-1888)), solution should be stored in dark at 4° C. until ready for use;
t. H₂ O₂ (30% solution) (Fisher catalog no. H325);
u. ABTS/H₂ O₂ (15 ml ABTS solution, 2 μl H₂ O₂) prepared 5 minutes before use and left at room temperature;
v. 0.2M HCl stock in H₂ O;
w. dimethylsulfoxide (100%)(Sigma Catalog No. D-8418); and
y. Trypsin-EDTA (Gibco BRL Catalog No. 25200-049).
   Protocol. The Following Protocol was used for Conducting the Assay:
1. Coat Corning 96-well elisa plates with 1.0 μg per well Cappel Anti-rabbit IgG antibody in 0.1M Na₂ CO₃ pH 9.6. Bring final volume to 150 μl per well. Coat plates overnight at 4° C. Plates can be kept up to two weeks when stored at 4° C.
2. Grow cells in Growth media(DMEM, supplemental with 2.0 mM L-Glutamine, 10% FBS) in suitable culture dishes until confluent at 37° C., 5% CO₂.
3. Harvest cells by trypsinization and seed in Corning 25850 polystyrene 96-well roundbottom cell plates, 25.000 cells/well in 200 μl of growth media.
4. Grow cells at least one day at 37° C., 5% CO₂.
5. Wash cells with D-PBS 1×.
6. Add 200 μl/well of starvation media (DMEM, 2.0 mM I-Glutamine, 0.1% FBS). Incubate overnight at 37° C., 5% CO₂.
7. Dilute Compounds/Extracts 1:20 in polypropylene 96 well plates using starvation media. Dilute dimethylsulfoxide 1:20 for use in control wells.
8. Remove starvation media from 96 well cell culture plates and add 162 μl of fresh starvation media to each well.
9. Add 18 μl of 1:20 diluted Compound/Extract dilution (from step 7) to each well plus the 1:20 dimethylsulfoxide dilution to the control wells (+/−VEGF), for a final dilution of 1:200 after cell stimulation. Final dimethylsulfoxide is 0.5%. Incubate the plate at 37° C., 5% CO₂ for two hours.
10. Remove unbound antibody from ELISA plates by inverting plate to remove liquid. Wash 3 times with TBSW +0.5% ethanolamine, pH 7.0. Pat the plate on a paper towel to remove excess liquid and bubbles.
11. Block plates with TBSW +0.5% ethanolamine, pH 7.0, 150 μl per well. Incubate plate thirty minutes while shaking on a microtiter plate shaker.
12. Wash plate 3 times as described in step 10.
13. Add 0.5 μg/well affinity purified anti-FLU-1 polyclonal rabbit antiserum. Bring final volume to 150 μl/well with TBSW +0.5% ethanolamine pH 7.0. Incubate plate for thirty minutes while shaking.
14. Add 180 μl starvation medium to the cells and stimulate cells with 20 μl/well 10.0 mM sodium ortho vanadate and 500 ng/ml VEGF (resulting in a final concentration of 1.0 mM sodium ortho vanadate and 50ng/ml VEGF per well) for eight minutes at 37° C., 5% CO₂. Negative control wells receive only starvation medium.
15. After eight minutes, media should be removed from the cells and washed one time with 200 μl /well PBS.
16. Lyse cells in 150 μl/well HNTG while shaking at room temperature for five minutes. HNTG formulation includes sodium ortho vanadate, sodium pyro phosphate and EDTA.
17. Wash ELISA plate three times as described in step 10.
18. Transfer cell lysates from the cell plate to elisa plate and incubate while shaking for two hours. To transfer cell lysate pipette up and down while scrapping the wells.
19. Wash plate three times as described in step 10.
20. Incubate ELISA plate with 0.02 μg/well UB40 in TBSW +05% ethanolamine. Bring final volume to 150 μl/well. Incubate while shaking for 30 minutes.
21. Wash plate three times as described in step 10.
22. Incubate ELISA plate with 1:10,000 diluted EIA grade goat anti-mouse IgG conjugated horseradish peroxidase in TBSW +0.5% ethanolamine, pH 7.0. Bring final volume to 150 μl/well. Incubate while shaking for thirty minutes.
23. Wash plate as described in step 10.
24. Add 100 μl of ABTS/H₂ O₂ solution to well. Incubate ten minutes while shaking.
25. Add 100 μl of 0.2M HCl for 0.1M HCl final to stop the color development reaction. Shake 1 minute at room temperature. Remove bubbles with slow stream of air and read the ELISA plate in an ELISA plate reader at 410 nm.

### HER-2 ELISA

Assay 1 EGF Receptor-HER2 Chimeric Receptor Assay In Whole Cells. HER2 kinase activity in hole EGFR-NIH3T3 cells was measured as described below:

Materials and Reagents. The Following Materials and Reagents were Used to Conduct the Assay:

a. EGF: stock concentration=16.5 ILM; EGF 201, TOYOBO, Co., Ltd. Japan.
b. 05-101 (UBI) (a monoclonal antibody recognizing an EGFR extracellular domain).
c. Anti-phosphotyrosine antibody (anti-Ptyr) (polyclonal)(see, Fendley, et al., supra).
d. Detection antibody: Goat anti-rabbit IgG horse radish peroxidase conjugate, TAGO, Inc., Burlingame, Calif.

e. TBST buffer:

| | | |
|---|---|---|
| Tris-HCl, pH 7.2 | 50 | mM |
| NaCl | 150 | mM |
| Triton X-100 | 0.1 | |

f. HNTG 5× stock:

| HEPES | 0.1M |
|---|---|
| NaCl | 0.75M |
| Glycerol | 50% |
| Triton X-100 | 1.0% |

g. ABTS stock:

| | | |
|---|---|---|
| Citric Acid | 100 | mM |
| Na₂HPO₄ | 250 | mM |
| HCl, conc. | 0.5 | pM |
| ABTS* | 0.5 | mg/ml |

*(2,2azinobis(3-ethylbenzthiazolinesulfonic acid)). Keep solution in dark at 4° C. until use.

h. Stock reagents of:
EDTA 100 mM pH 7.0
Na₃ VO₄ 0.5M
Na₄ (P₂ O₇) 0.2M
   Procedure. The Following Protocol was Used:
   A. Pre-coat ELISA Plate
1. Coat ELISA plates (Corning, 96 well, Cat. #25805-96) with 05-101 antibody at 0.5 g per well in PBS, 100 μl final volume/well, and store overnight at 4° C. Coated plates are good for up to 10 days when stored at 4° C.
2. On day of use, remove coating buffer and replace with 100 μl blocking buffer (5% Carnation Instant Non-Fat Dry Milk in PBS). Incubate the plate, shaking, at room temperature (about 23° C. to 25° C.) for 30 minutes. Just prior to use, remove blocking buffer and wash plate 4 times with TBST buffer.
   B. Seeding Cells
1. An NIH3T3 cell line overexpressing a chimeric receptor containing the EGFR extracellular domain and extracellular HER2 kinase domain can be used for this assay.
2. Choose dishes having 80-90% confluence for the experiment. Trypsinize cells and stop reaction by adding 10% fetal bovine serum. Suspend cells in DMEM medium (10% CS DMEM medium) and centrifuge once at 1500 rpm, at room temperature for 5 minutes.
3. Resuspend cells in seeding medium (DMEM, 0.5% bovine serum), and count the cells using trypan blue. Viability above 90% is acceptable. Seed cells in DMEM medium (0.5% bovine serum) at a density of 10,000 cells per well, 100 μl per well, in a 96 well microtiter plate. Incubate seeded cells in 5% CO₂ at 37° C. for about 40 hours.
   C. Assay Procedures
1. Check seeded cells for contamination using an inverted microscope. Dilute drug stock (10 mg/ml in DMSO) 1:10 in DMEM medium, then transfer 5 l to a TBST well for a final drug dilution of 1:200 and a final DMSO concentration of 1%. Control wells receive DMSO alone. Incubate in 5% CO₂ at 37° C. for two hours.
2. Prepare EGF ligand: dilute stock EGF in DMEM so that upon transfer of 10 μl dilute EGF (1:12 dilution), 100 nM final concentration is attained.

3. Prepare fresh HNTG* sufficient for 100 μl per well; and place on ice.

HNTG* (10 ml):

| | |
|---|---|
| HNTG stock | 2.0 ml |
| milli-Q H₂O | 7.3 ml |
| EDTA, 100 mM, pH 7.0 | 0.5 ml |
| Na₃VO₄, 0.5M | 0.1 ml |
| Na₄(P₂O₇), 0.2M | 0.1 ml |

4. After 120 minutes incubation with drug, add prepared SGF ligand to cells, 10 μl per well, to a final concentration of 100 nM. Control wells receive DMEM alone. Incubate, shaking, at room temperature, for 5 minutes.
5. Remove drug, EGF, and DMEM. Wash cells twice with PBS. Transfer HNTG* to cells, 100 μl per well. Place on ice for 5 minutes. Meanwhile, remove blocking buffer from other ELISA plate and wash with TBST as described above.
6. With a pipette tip securely fitted to a micropipettor, scrape cells from plate and homogenize cell material by repeatedly aspirating and dispensing the HNTG* lysis buffer. Transfer lysate to a coated, blocked, and washed ELISA plate. Incubate shaking at room temperature for one hour.
7. Remove lysate and wash 4 times with TBST. Transfer freshly diluted anti-Ptyr antibody to ELISA plate at 100 μl per well. Incubate shaking at room temperature for 30 minutes in the presence of the anti-Ptyr antiserum (1:3000 dilution in TBST).
8. Remove the anti-Ptyr antibody and wash 4 times with TBST. Transfer the freshly diluted TAGO anti-rabbit IgG antibody to the ELISA plate at 100 μl per well. Incubate shaking at room temperature for 30 minutes (anti-rabbit IgG antibody: 1:3000 dilution in TBST).
9. Remove TAGO detection antibody and wash 4 times with TBST. Transfer freshly prepared ABTS/H₂ O₂ solution to ELISA plate, 100 μl per well. Incubate shaking at room temperature for 20 minutes. (ABTS/H₂ O₂ solution: 1.0 μl 30% H₂O₂ in 10 ml ABTS stock).
10. Stop reaction by adding 50 μl 5N H₂ SO₄ (optional), and determine O.D. at 410 nm.
11. The maximal phosphotyrosine signal is determined by subtracting the value of the negative controls from the positive controls. The percent inhibition of phosphotyrosine content for extract-containing wells is then calculated, after subtraction of the negative controls.
   Assay 2: HER-2-BT474 ELISA. A Second Assay May be Conducted to Measure Whole Cell HER2 Activity. Such Assay may be Conducted as Follows:
   Materials And Reagents. The Following Materials and Reagents were Used:
a. BT-474 (ATCC HBT20), a human breast tumor cell line which expresses high levels of HER2 kinase.
b. Growth media comprising RPMI+10% FBS+GMS-G (Gibco supplement)+glutamine for use in growing BT-474 in an incubator with 5% CO₂ at 37° C.
c. A monoclonal anti-HER2 antibody.

d. D-PBS:

| | |
|---|---|
| KH₂HPO₄ | 0.20 g/l 10 (GIBCO, 310-4190AJ) |
| K₂HPO₄ | 2.16 g/l |
| KCl | 0.20 g/l |
| NaCl | 8.00 g/l (pH 7.2) |

e. Blocking Buffer: TBST plus 5% Milk (Carnation Instant Non-Fat Dry Milk).

f. TBST buffer:

| Tris-HCl | 50 mM |
|---|---|
| NaCl | 150 mM (pH 7.2, HCl 10 N) |
| Triton X-100 | 0.1% |

wherein stock solution of TES (10×) is prepared, and Triton X-100 is added to the buffer during dilution.

g. HNTG buffer (5×):

| HEPES | 0.1M |
|---|---|
| NaCl | 750 mM (pH 7.2 (HCl, 10 N) |
| Glycerol | 50% |
| Triton X-100 | 1.0% |

Stock solution (5×) is prepared and kept in 40° C.
h. EDTA-HCl: 0.5M pH 7.0 (10N HCl) as 500× stock.
i. Na₃VO₄: 0.5M as 100× stock is kept at −80° C. as aliquots.
j. Na₄(P₂ O₇): 0.2M as 100× stock.
k. Polyclonal antiserum anti-phosphotyrosine.
l. Goat anti-rabbit IgG, horseradish peroxidase (POD) conjugate (detection antibody), Tago (Cat. No. 4520; Lot No. 1802): Tago, Inc., Burlingame, Calif.

m. ABTS solution:

| | | |
|---|---|---|
| Citric acid | 100 | mM |
| Na₂HPO₄ | 250 | mM (pH 4.0, 1N HCl) |
| ABTS | 0.5 | mg/ml |

wherein ABTS is 2.2′-azinobis(3-ethylbenzthiazoline sulfonic acid). For this assay, the ABTS solution should be kept in the dark at 4° C. The solution should be discarded when it turns green.
n. Hydrogen peroxide: 30% solution is kept in dark and 4° C.
Procedure. All the following steps are at room temperature and aseptically performed, unless stated otherwise. All ELISA plate washing is by rinsing with distilled water three times and once with TBST.
   A. Cell Seeding
1. Grow BT474 cells in tissue culture dishes (Corning 25020-100) to 80-90% confluence and collect using Trypsin-EDTA (0.25%, GIBCO).
2. Resuspend the cells in fresh medium and transfer to 96-well tissue culture plates (Corning, 25806-96) at about 25,000-50,000 cells/well (100 μl/well) Incubate the cells in 5% CO₂ at 37° C. overnight.
   B. ELISA Plate Coating and Blocking
1. Coat the ELISA plate (Corning 25805-96) with anti HER2 antibody at 0.5 μg/well in 150 μl PBS overnight at 4° C., and seal with parafilm. The antibody coated plates can be used up to 2 weeks, when stored at 4° C.
2. On the day of use, remove the coating solution, replace with 200 μl of Blocking Buffer, shake the plate, and then remove the blocking buffer and wash the plate just before adding lysate.
   C. Assay Procedures
1. TBST the drugs in serum-free condition. Before adding drugs, the old media is replaced with serum-free RPMI (90 μl/well).
2. Dilute drug stock (in 100% DMSO) 1:10 with RPMI, and transfer 10 μl/well of this solution to the cells to achieve a final drug DMSO concentration at 1%. Incubate the cells in 5% CO₂ at 37° C.

3. Prepare fresh cell lysis buffer (HNTG*)

| | |
|---|---|
| 5xHNTG | 2 ml |
| EDTA | 0.2 ml |
| Na₃VO₄ | 0.1 ml |
| Na₄P₂O₇ | 0.1 ml |
| H₂O | 7.3 ml |

4. After drug preincubation for two hours remove all the solution from the plate, transfer HNTG* (100 μl/well) to the cells, and shake for 10 minutes.
5. Use a 12-channel pipette to scrape the cells from the plate, and homogenize the lysate by repeat aspiration and dispensing. Transfer all the lysate to the ELISA plate and shake for 1 hour.
6. Remove the lysate, wash the plate, add anti-pTyr (1:3,000 with TBST) 100 μl/well, and shake for 30 minutes.
7. Remove anti-pTyr, wash the plate, add goat anti-rabbit IgG conjugated antibody (1:5,000 with TBST) 100 μl/well, and shake for 30 minutes.
8. Remove anti-rabbit IgG antibody, wash the plate, and add fresh ABTS/H₂ O₂ (1.2 μl H₂ O₂ to 100 ml ABTS) 100 l/well to the plate to start color development, which usually takes 20 minutes.
9. Measure OD 410 nM, Dynatec MR5000.

### PDGF-R ELISA

All cell culture media, glutamine, and fetal bovine serum were purchased from Gibco Life Technologies (Grand Island, N.Y.) unless otherwise specified. All cells were grown in a humid atmosphere of 90-95% air and 5-10% CO₂ at 37° C. All cell lines were routinely subcultured twice a week and were negative for mycoplasma as determined by the Mycotect method (Gibco).

For ELISA assays, cells (U1242, obtained from Joseph Schlessinger, NYU) were grown to 80-90% confluency in growth medium (MEM with 10% FBS, NEAA, 1 mM NaPyr and 2 mM GLN) and seeded in 96-well tissue culture plates in 0.5% serum at 25,000 to 30,000 cells per well. After overnight incubation in 0.5% serum-containing medium, cells were changed to serum-free medium and treated with test compound for 2 hr in a 5% CO₂, 37° C. incubator. Cells were then stimulated with ligand for 5-10 minutes followed by lysis with HNTG (20 mM Hepes, 150 mM NaCl, 10% glycerol, 5 mM EDTA, 5 mM Na₃ VO₄, 0.2% Triton X-100, and 2 mM NaPyr). Cell lysates (0.5 mg/well in PBS) were transferred to ELISA plates previously coated with receptor-specific antibody and which had been blocked with 5% milk in TBST (50 mM Tris-HCl pH 7.2, 150 mM NaCl and 0.1% Triton X-100) at room temperature for 30 min. Lysates were incubated with shaking for 1 hour at room temperature. The plates were washed with TBST four times and then incubated with polyclonal anti-phosphotyrosine antibody at room temperature for 30 minutes. Excess anti-phosphotyrosine antibody was removed by rinsing the plate with TBST four times. Goat anti-rabbit IgG antibody was added to the ELISA plate for 30 min at room temperature followed by rinsing with TBST four more times. ABTS (100 mM citric acid, 250 mM Na₂ HPO₄ and 0.5 mg/mL 2,2′-azino-bis(3-ethylbenzthiazoline-6-sulfonic acid)) plus H₂ O₂ (1.2 mL 30% H₂ O₂ to 10 ml ABTS) was added to the ELISA plates to start color development. Absorbance at 410 nm with a reference wavelength of 630 nm was recorded about 15 to 30 min after ABTS addition.

### IGF-I ELISA

The following protocol may be used to measure phosphotyrosine level on IGF-I receptor, which indicates IGF-I receptor tyrosine kinase activity.

Materials And Reagents. The Following Materials and Reagents were Used:

a. The cell line used in this assay is 3T3/IGF-1R, a cell line which overexpresses IGF-1 receptor.
b. NIH3T3/IGF-1R is grown in an incubator with 5% CO₂ at 37° C. The growth media is DMEM+10% FBS (heat inactivated)+2 mM L-glutamine.
c. Anti-IGF-1R antibody named 17-69 is used. Antibodies are purified by the Enzymology Lab, SUGEN, Inc.

d. D-PBS:

| | |
|---|---|
| KH₂PO₄ | 0.20 g/l |
| K₂HPO₄ | 2.16 g/l |
| KCl | 0.20 g/l |
| NaCl | 8.00 g/l (pH 7.2) |

e. Blocking Buffer: TBST plus 5% Milk (Carnation Instant Non-Fat Dry Milk).

f. TBST buffer:

| | | |
|---|---|---|
| Tris-HCl | 50 | mM |
| NaCl | 150 | mM (pH 7.2/HCl 10N) |

| | |
|---|---|
| Triton X-100 | 0.1% |

Stock solution of TBS (10×) is prepared, and Triton X-100 is added to the buffer during dilution.

g. HNTG buffer:

| | | |
|---|---|---|
| HEPES | 20 | mM |
| NaCl | 150 | mM (pH 7.2/HCl 1N) |

| | |
|---|---|
| Glycerol | 10% |
| Triton X-100 | 0.2% |

Stock solution (5×) is prepared and kept at 4° C.
h. EDTA/HCl: 0.5M pH 7.0 (NaOH) as 100× stock.
i. Na₃VO₄: 0.5M as 100× stock and aliquots are kept in −80° C.
j. Na₄P₂O₇: 0.2M as 100× stock.
k. Insulin-like growth factor-1 from Promega (Cat# G5111).
l. Polyclonal antiserum anti-phosphotyrosine: rabbit sera generated by Enzymology Lab., SUGEN Inc.
m. Goat anti-rabbit IgG, POD conjugate (detection antibody), Tago (Cat. No. 4520, Lot No. 1802): Tago, Inc., Burlingame, Calif.

n. ABTS (2.2′-azinobis(3-ethylbenzthiazolinesulfonic acid)) solution:

| | | |
|---|---|---|
| Citric acid | 100 | mM |
| Na₂HPO₄ | 250 | mM (pH 4.0/1 N HCl) |
| ABTS | 0.5 | mg/ml |

ABTS solution should be kept in dark and 4° C. The solution should be discarded when it turns green.
o. Hydrogen Peroxide: 30% solution is kept in the dark and at 4° C.
Procedure. All the following steps are conducted at room temperature unless it is specifically indicated. All ELISA plate washings are performed by rinsing the plate with tap water three times, followed by one TBST rinse. Pat plate dry with paper towels.
   A. Cell Seeding:
1. The cells, grown in tissue culture dish (Corning 25020-100) to 80-90% confluence, are harvested with Trypsin-EDTA (0.25%, 0.5 ml/D-100, GIBCO).
2. Resuspend the cells in fresh DMEM+10% FBS+2 mM L-Glutamine, and transfer to 96-well tissue culture plate (Corning, 25806-96) at 20,000 cells/well (100 μl/well). Incubate for 1 day then replace medium to serum-free medium (90 μl) and incubate in 5% CO₂ and 37° C. overnight.
   B. ELISA Plate Coating and Blocking:
1. Coat the ELISA plate (Corning 25805-96) with Anti-IGF-IR antibody at 0.5 μg/well in 100 μl PBS at least 2 hours.
2. Remove the coating solution, and replace with 100 μl Blocking Buffer, and shake for 30 minutes. Remove the blocking buffer and wash the plate just before adding lysate.
   C. Assay Procedures:
1. The drugs are tested in serum-free condition.
2. Dilute drug stock (in 100% DMSO) 1:10 with DMEM in 96-well polypropylene plate, and transfer 10 μwell of this solution to the cells to achieve final drug dilution 1:100, and final DMSO concentration of 1.0%. Incubate the cells in 5% CO₂ at 37° C. for 2 hours.

3. Prepare fresh cell lysis buffer (HNTG*).

| | |
|---|---|
| HNTG | 2 ml |
| EDTA | 0.1 ml |
| Na₃VO₄ | 0.1 ml |
| Na₄(P₂O₇) | 0.1 ml |
| H₂O | 7.3 ml |

4. After drug incubation for two hours, transfer 10 μl/well of 200 nM IGF-1 Ligand in PBS to the cells (Final Conc.=20 nM), and incubate at 5% CO₂ at 37° C. for 10 minutes.
5. Remove media and add 100 μl/well HNTG* and shake for 10 minutes. Look at cells under microscope to see if they are adequately lysed.
6. Use a 12-channel pipette to scrape the cells from the plate, and homogenize the lysate by repeat aspiration and dispense. Transfer all the lysate to the antibody coated ELISA plate, and shake for 1 hour.
7. Remove the lysate, wash the plate, transfer anti-pTyr (1:3,000 with TBST) 100 μl/well, and shake for 30 minutes.
8. Remove anti-pTyr, wash the plate, transfer Tago (1:3,000 with TBST) 100 μl/well, and shake for 30 minutes.
9. Remove detection antibody, wash the plate, and transfer fresh ABTS/H₂ O₂ (1.2 μl H₂ O₂ to 10 ml ABTS) 100 μl/well to the plate to start color development.
10. Measure OD in Dynatec MR5000, which is connected to Ingres.

### EGF Receptor ELISA

EGF Receptor kinase activity (EGFR-NIH3T3 assay) in whole cells was measured as described below:

Materials and Reagents. The Following Materials and Reagents were Used

a. EGF Ligand: stock concentration=16.5 μM; EGF 201, TOYOBO, Co., Ltd. Japan.
b. 05-101 (UBI) (a monoclonal antibody recognizing an EGFR extracellular domain).
c. Anti-phosphotyosine antibody (anti-Ptyr) (polyclonal).
d. Detection antibody: Goat anti-rabbit IgG horse radish peroxidase conjugate, TACO, Inc., Burlingame, Calif.

e. TBST buffer:

| Tris-HCl, pH 7 | 50 mM |
|---|---|
| NaCl | 150 mM |
| Triton X-100 | 0.1 |

f. HNTG 5× stock:

| HEPES | 0.1M |
|---|---|
| NaCl | 0.75M |
| Glycerol | 50 |
| Triton X-100 | 1.0% |

g. ABTS stock:

| | | |
|---|---|---|
| Citric Acid | 100 | mM |
| Na₂HPO₄ | 250 | mM |
| HCl, conc. | 4.0 | pH |
| ABTS* | 0.5 | mg/ml |

Keep solution in dark at 4° C. until used.
h. Stock reagents of:
EDTA 100 mM pH 7.0
Na₃VO₄ 0.5M
Na₄(P₂O₇) 0.2M
Procedure. The following protocol was used:
   A. Pre-Coat ELISA Plate
1. Coat ELISA plates (Corning, 96 well, Cat. #25805-96) with 05-101 antibody at 0.5 μg per well in PBS, 150 μl final volume/well, and store overnight at 4° C. Coated plates are good for up to 10 days when stored at 4° C.
2. On day of use, remove coating buffer and replace with blocking buffer (5% Carnation Instant NonFat Dry Milk in PBS). Incubate the plate, shaking, at room temperature (about 23° C. to 25° C.) for 30 minutes. Just prior to use, remove blocking buffer and wash plate 4 times with TBST buffer.
   B. Seeding Cells
1. NIH 3T3/C7 cell line (Honegger, et al., Cell 51:199-209,1987) can be use for this assay.
2. Choose dishes having 80-90% confluence for the experiment. Trypsinize cells and stop reaction by adding 10% CS DMEM medium. Suspend cells in DMEM medium (10% CS DMEM medium) and centrifuge once at 1000 rpm, and once at room temperature for 5 minutes.
3. Resuspend cells in seeding medium (DMEM, 0.5% bovine serum), and count the cells using trypan blue. Viability above 90% is acceptable. Seed cells in DMEM medium (0.5% bovine serum) at a density of 10,000 cells per well, 100 μl per well, in a 96 well microtiter plate. Incubate seeded cells in 5% Co₂ at 37° C. for about 40 hours.
   C. Assay Procedures.
1. Check seeded cells for contamination using an inverted microscope. Dilute drug stock (10 mg/ml in DMSO) 1:10 in DMEM medium, then transfer 5 μl to a test well for a final drug dilution of 1:200 and a final DMSO concentration of 1%. Control wells receive DMSO alone. Incubate in 5% CO₂ at 37° C. for one hour.
2. Prepare EGF ligand: dilute stock EGF in DMEM so that upon transfer of 10 μl dilute EGF (1:12 dilution), 25 nM final concentration is attained.
3. Prepare fresh 10 ml HNTG* sufficient for 100 μl per well wherein HNTG* comprises: HNTG stock (2.0 ml), milli-Q H₂ O (7.3 ml), EDTA, 100 mM, pH 7.0 (0.5 ml), Na₃ VO₄ 0.5M (0.1 ml) and Na₄ (P₂O₇), 0.2M (0.1 ml).
4. Place on ice.
5. After two hours incubation with drug, add prepared EGF ligand to cells, 10 μl per well, to yield a final concentration of 25 nM. Control wells receive DMEM alone. Incubate, shaking, at room temperature, for 5 minutes.
6. Remove drug, EGF, and DMEM. Wash cells twice with PBS. Transfer HNTG*to cells, 100 μl per well. Place on ice for 5 minutes. Meanwhile, remove blocking buffer from other ELISA plate and wash with TBST as described above.
7. With a pipette tip securely fitted to a micropipettor, scrape cells from plate and homogenize cell material by repeatedly aspirating and dispensing the HNTG* lysis buffer. Transfer lysate to a coated, blocked, and washed ELISA plate. Incubate shaking at room temperature for one hour.
8. Remove lysate and wash 4 times with TBST. Transfer freshly diluted anti-Ptyr antibody to ELISA plate at 100 μl per well. Incubate shaking at room temperature for 30 minutes in the presence of the anti-Ptyr antiserum (1:3000 dilution in TBST).
9. Remove the anti-Ptyr antibody and wash 4 times with TBST. Transfer the freshly diluted TAGO 30 anti-rabbit IgG antibody to the ELISA plate at 100 μl per well. Incubate shaking at room temperature for 30 minutes (anti-rabbit IgG antibody: 1:3000 dilution in TBST).
10. Remove detection antibody and wash 4 times with TBST. Transfer freshly prepared ABTS/H₂ O₂ solution to ELISA plate, 100 μl per well. Incubate at room temperature for 20 minutes. ABTS/H₂ O₂ solution: 1.2 μl 30% H₂ O₂ in 10 ml ABTS stock.
11. Stop reaction by adding 50 μl N H₂ SO₄ (optional), and determine O.D. at 410 nm.
12. The maximal phosphotyrosine signal is determined by subtracting the value of the negative controls from the positive controls. The percent inhibition of phosphotyrosine content for extract-containing wells is then calculated, after subtraction of the negative controls.

### Cellular Insulin Receptor ELISA

The following protocol was used to determine whether the compounds of the present invention possessed insulin receptor tyrosine kinase activity.

Materials And Reagents. The Following Materials and Reagents were Used to Measure Phophotyrosine Levels on the Insulin Receptor (Indicating Insulin Receptor Tyrosine Kinase Activity):

1. The preferred cell line was an NIH3T3 cell line (ATCC No. 1658) which overexpresses Insulin Receptor (H25 cells);
2. H25 cells are grown in an incubator with 5% CO₂ at 37° C. The growth media is DMEM+10% FBS (heat inactivated)+2 mm L-Glutamine;
3. For ELISA plate coating, the monoclonal anti-IR antibody named BBE is used. Said antibodies was purified by the Enzymology Lab, SUGEN, Inc.;

4. D-PBS, comprising:

| | | |
|---|---|---|
| KH₂PO₄ | 0.20 | g/l (GIBCO, 310-4190AJ) |
| K₂HPO₄ | 2.16 | g/l |
| KCl | 0.20 | g/l |
| NaCl | 8.00 | g/l (pH 7.2); |

5. Blocking Buffer: TBST plus 5% Milk (Carnation Instant Non-Fat Dry Milk);

6. TBST buffer, comprising:

| | | |
|---|---|---|
| Tris-HCl | 50 | mM |
| NaCl | 150 | mM pH 7.2 (HCl, 1 N) |

| | |
|---|---|
| Triton X-100 | 0.1% |

Note: Stock solution of TBS (10×) is prepared, and Triton X-100 is added to the buffer during dilution;

7. HNTG buffer, comprising:

| | | |
|---|---|---|
| HEPES | 20 | mM |
| NaCl | 150 | mM pH 7.2 (HCl, 1 N) |

| | |
|---|---|
| Glycerol | 10% |
| Triton X-100 | 0.2% |

Note: Stock solution (5×) is prepared and kept at 4° C.
8. EDTA HCl: 0.5M pH 7.0 (NaOH) as 100× stock;
9. Na₃VO₄: 0.5M as 100× stock and aliquots are kept in −80° C.;
10. Na₄P₂O₇: 0.2M as 100× stock;
11. Insulin from GIBCO BRL (Cat# 18125039);
12. Polyclonal antiserum Anti-phosphotyrosine: rabbit sera generated by Enzymology Lab., SUGEN Inc.;
13. Detection antibody, preferably goat anti-rabbit IgG, POD conjugate, Tago (Cat. No. 4520: Lot No. 1802): Tago, Inc., Burlingame, Calif.;

14. ABTS solution, comprising:

| | |
|---|---|
| Citric acid | 100 mM |
| Na₂HPO₄ | 250 mM pH 4.0 (1 N HCl) |
| ABTS | 0.5 mg/ml |

wherein ABTS is 2,2′-azinobis (3-ethylbenathiazoline sulfonic acid) and stored in the dark at 4° C. and discarded when it turns green.
15. Hydrogen Peroxide: 30% solution is kept in the dark and at 40° C. Protocol. All the following steps are conducted at room temperature unless it is specifically indicated. All ELISA plate washings are performed by rinsing the plate with tap water three times, followed by one TBST rinse. All plates were tapped dry with paper towels prior to use.
   A. Cell Seeding:
1. The cells were grown in tissue culture dish (10 cm, Corning 25020-100) to 80-90% confluence and harvested with Trypsin-EDTA (0.25%, 0.5 ml/D-100, GIBCO);
2. Resuspend the cells in fresh DMEM+10% FBS+2 mM L-Glutamine, and transfer to 96-well tissue culture plate (Corning, 25806-96) at 20,000 cells/well (100 μl/well). The cells are then incubated for 1 day. Following such incubation, 0.01% serum medium (90 μl) replaces the old media and the cells incubate in 5% CO₂ and 37° C. overnight.
   B. ELISA Plate Coating and Blocking:
1. Coat the ELISA plate (Corning 25805-96) with Anti-IR Antibody at 0.5 μg/well in 100 μl PBS at least 2 hours.
2. Remove the coating solution, and replace with 100 μl blocking Buffer, and shake for 30 minutes. Remove the blocking buffer and wash the plate just before adding lysate.
   C. Assay Procedures
1. The drugs are tested in serum-free condition.
2. Dilute drug stock (in 100% DMSO) 1:10 with DMEM in 96-well poly-propylene plate, and transfer 10 μl /well of this solution to the cells to achieve final drug dilution 1:100, and final DMSO concentration of 1.0%. Incubate the cells in 5% CO₂ at 37° C. for 2 hours.

3. Prepare fresh cells lysis buffer (HNTG*)

| | |
|---|---|
| HNTG (5x) | 2 ml |
| EDTA | 0.1 ml |
| Na₃VO₄ | 0.1 ml |
| Na₄P₂O₇ | 0.1 ml |
| H₂O | 7.3 ml |
| HNTG* | 10 ml |

4. After drug incubation for two hours, transfer 10 μl/well of 1 μM insulin in PBS to the cells (Final concentration=100 nM), and incubate at 5% CO₂ at 37° C. for 10 minutes.
5. Remove media and add 100 μl/well HNTG* and shake for 10 minutes. Look at cells under microscope to see if they are adequately lysed.
6. Using a 12-channel pipette, scrape the cells from the plate, and homogenize the lysate by repeat aspiration and dispense. Transfer all the lysate to the antibody coated ELISA plate, and shake for 1 hour.
7. Remove the lysate, wash the plate, transfer anti-pTyr (1:3,000 with TBST) 100 μl/well, and shake for 30 minutes.
8. Remove anti-pTyr, wash the plate, transfer Tago (1:3,000 with TBST) 100 μl/well, and shake for 30 minutes.
9. Remove detection antibody, wash the plate, and transfer fresh ABTS/H₂ O₂ (1.2 μl H₂ O₂ to 10 ml ABTS) 100 μl/well to the plate to start color development. 10. Measure OD in Dynatec MR5000, which is connected to Ingres. All following steps should follow Ingres instruction.

### Experimental Results From ELISA Assays

The Experimental Results for Various Compounds According to the Invention Using the Above-Described Protocols are Set Forth at Table 1:

TABLE 1
ELISA Assay Results

| | | | | HER2 | |
|---|---|---|---|---|---|
| COM- | PDGFR | FLK-1 | EGFR | Kinase | IGF-1R |
| POUND | IC50(μM) | IC50(μM) | IC50(μM) | IC50(μM) | IC50(μM) |
| SU4312 | 19.4 | 0.8 | | | |
| SU4313 | 14.5 | 18.8 | 11 | 16.9 | 8.0 |
| SU4314 | 12 | 0.39 | | | |
| SU4793 | 87.4 | 4.2 | | | |
| SU4794 | | 11.8 | | | |
| SU4798 | | 28.8 | | | |
| SU4799 | | 9 | | | |
| SU4932 | | 2.2 | | | |
| SU4944 | | 8.5 | | | |
| SU4952 | | 22.6 | | | |
| SU4956 | | | | 22.5 | |
| SU4967 | 7.9 | 11.2 | | | |
| SU4979 | | 20.9 | | | |
| SU4981 | 33.1 | 2.1 | | | |
| SU4982 | | 21.6 | | 39.4 | |
| SU4983 | | 4.1 | | | |
| SU4984 | 5.8 | 1.6 | | 90.2 | |
| SU5204 | | 4 | | 51.5 | |
| SU5205 | | 9.6 | | | |
| SU5208 | | 4.7 | | | |
| SU5214 | | 14.8 | 36.7 | | |
| SU5218 | | 6.4 | | | |
| SU5401 | | 2.9 | | 89.8 | |
| SU5402 | | 0.4 | | | |
| SU5403 | | 1.8 | | | |
| SU5404 | 17 | 0.24 | | | |
| SU5405 | | 23.8 | | | |
| SU5406 | | 0.17 | | | |
| SU5407 | 53.7 | 1.1 | | | |
| SU5408 | | 0.07 | | | |
| SU5416 | 10.8 | 0.11 | | | |
| SU5418 | | 15.4 | | | |
| SU5419 | | 2.3 | | | |
| SU5421 | | 4.6 | | | |
| SU5424 | | 2.4 | | | |
| SU5425 | | 51.4 | | | |
| SU5427 | | 4.5 | | 70.6 | |
| SU5428 | | 8.6 | | | |
| SU5430 | | 73.4 | | | |
| SU5431 | | 41.2 | | | |
| SU5432 | | 22.8 | | | |
| SU5450 | | 4.5 | | 92.6 | |
| SU5451 | | 3.4 | 44 | | |
| SU5453 | 65.5 | 0.14 | | | |
| SU5455 | | 36.2 | | | |
| SU5463 | | 0.18 | | | |
| SU5464 | | 20.3 | | | |
| SU5466 | 86 | 1.6 | | | |
| SU5468 | 55.9 | 2.7 | | | |
| SU5472 | | 8.7 | | | |
| SU5473 | 14.2 | 1.5 | | | |
| SU5474 | | 7.4 | | | |
| SU5477 | | 0.15 | | | |
| SU5480 | | 5.3 | 39.6 | 30.4 | |

### Cell Growth Assays

The following assays may be conducted to measure the effect of the claimed compounds and combinations upon cell growth as a result of the compound's interaction with one or more RTKs. Unless otherwise specified, the following assays may be generally applied to measure the activity of a compound against any particular RTK. To the extent that an assay, set forth below, refers to a specific RTK, one skilled in the art would be able to adapt the disclosed protocol for use to measure the activity of a second RTK.

### Soft Agar Assay

The soft agar assay may be used to measure the effects of substances or combinations containing said substances on cell growth. Unless otherwise stated the soft agar assays were carried out as follows:

Material And Reagents. The Following Materials and Reagents were Used:

a. A water bath set at 39° C. and another water bath at 37° C.
b. 2× assay medium is comprised of 2× Dulbecco's 5Modified Eagle's Medium (DMEM) (Gibco Cat. # CA400-4AN03) supplemented by the following: 20% Fetal Bovine Serum (FBS), 2 mM sodium pyruvate, 4 mM glutamine amine; and 20 mM HEPES Non-essential Amino Acids (1:50 from 100× stock).
c. 1× assay medium made of 1× DMEM supplemented with 10% FBS, 1 mM sodium pyruvate, 2 mM glutamine, 10 mM HEPES, non-essential amino acid (1:100 from 100× stock).
d. 1.6% SeaPlaque Agarose in autoclave bottle.
e. Sterile 35 mm Corning plates (FMC Bioproducts Cat. #50102).
f. Sterile 5 ml glass pipets (individually wrapped).
g. Sterile 15 ml and 50 ml conical centrifuge tubes.
h. Pipets and sterile tips.
i. Sterile microcentrifuge tubes.
j. Cells in T75 flasks: SKOV-3 (ATCC HTB77).
k. 0.25% Trypsin solution (Gibco #25200-015).
Procedure. The following procedure was used to onduct the soft agar assay:
   A. Procedure for Making the Base Layer
1. Have all the media warmed up in the 37° C. water bath.
2. To make 1× of assay medium+0.8% agar: make a 1:2 (vol:vol) dilution of melted agar (cooled to 39° C.), with 2× assay medium.
3. Keep all media with agar warm in the 39° C. water bath when not in use.
4. Dispense 1 ml of 1× assay medium+0.8% agar into dishes and gently swirl plate to form a uniform base layer. Bubbles should be avoided.
5. Refrigerate base layers to solidify (about 20 minutes). Base layers can be stored overnight in the refrigerator.
   B. Procedure for Collecting Cells
1. Take out one flask per cell line from the incubator; aspirate off medium; wash once with PBS and aspirate off; add 3 ml of trypsin solution.
2. After all cells dissociate from the flask, add 3 ml of 1× assay media to inhibit trypsin activity. Pipet the cells up and down, then transfer the suspension into a 15 ml tube.
3. Determine the concentration of cells using a Coulter counter, and the viability by trypan blue exclusion.
4. Take out the appropriate volume needed to seed 3300 viable cells per plate and dilute it to 1.5 ml with 1× assay medium.
   C. Procedure for Making the Upper 0.4% Agarose Layer:
1. Add TBST compounds at twice the desired final assay concentration;+1.5 ml of cell suspension in 1× assay medium 10% FBS;+1.5 ml of 1× assay medium+0.8% agarose*: Total=3.0 ml 1× media 10% FBS+0.4% agarose with 3300 viable cells/ml, with and without TBST compounds.
*(Made by 1:2 dilution of 2× media with 1.6% agar 30 for the base layer procedure above.)
2. Plate 1 ml of the Assay Mix onto the 1 ml base layer. The duplicates are plated from the 3 ml volume.
3. Incubate the dishes for 2-3 weeks in a 100% humidified, 10% CO₂ incubator.
4. Colonies that are 60 microns and larger are scored positive.

### Sulforhodamine B (SRB) Growth Assays

The SRB assays may be used to measure the effects of substances or cell growth. The assays are carried out as follows:

### Assay 1:3T3/E/H+TGF-a(T) Cell Growth SRB Assay

Materials:

96-well flat bottom sterile plates
96-well round bottom sterile plates
sterile 25 ml or 100 ml reservoir
pipets, multi-channel pipetman
sterile pipet tips
sterile 15 ml and 50 ml tubes
   Reagents:
0.4% SRB in 1% acetic acid
10 mM Tris base
10% TCA
1% acetic acid
sterile DMSO (Sigma)
compound in DMSO (100 mM or less stock solution)
25% Trypsin-EDTA in Cell Dissociation Solution (Sigma)
   Cell Line and Growth Medium:
3T3/E/H+TGF-a(T) (NIH 3T3 clone 7 cells expressing EGF-R/HER2 chimera and TGF-a, tumor-derived autocrine loop cells)
2% calf serum/DMEM+2 mM glutamine
   Protocol:
Day 0: Cell Plating:
This part of assay is carried out in a laminar flow hood.
1. Trypsinize cells as usual. Transfer 100 μl of cell suspension to 10 ml of isotone. Count cells with the Coulter Counter.
2. Dilute cells in growth medium to 60,000 cells/ml. Transfer 100 μl of cells to each well in a 96-well flat bottom plate to give 6000 cells/well.
3. Use half of plate (4 rows) for each compound and quadruplicate wells for each compound concentration, a set of 4 wells for medium control and 4 wells for DMSO control.
4. Gently shake plates to allow for uniform attachment of the cells.
5. Incubate the plates at 37° C. in a 10% CO2 incubator.
   Day 1: Addition of Compound:
This part of assay is carried out in a laminar flow hood.
1. In 96 well-round bottom plate, add 125 μl of growth medium to columns 3-11. This plate is used to titrate out the compound, 4 rows per compound.
2. In a sterile 15 ml tube, make a 2× solution of the highest concentration of compound by adding 8 μl of the compound to a total of 2 ml growth medium for a dilution of 1:250. At this dilution, the concentration of DMSO is 0.4% for a 2× solution or 0.2% for 1× solution on the cells. The starting concentration of the compound is usually 100 uM but this concentration may vary depending upon the solubility of the compound.
3. Transfer the 2× starting compound solution to quadruplicate wells in column 12 of the 96-well round bottom plate. Do 1:2 serial dilutions across the plate from right to left by transferring 125 μl from column 12 to column 11, column 11 to 10 and so on. Transfer 100 μl of compound dilutions onto 100 μl medium on cells in corresponding wells of 96-well flat bottom plate. Total volume per well should be 200 μl.
4. For vehicle control, prepare a 2× solution of DMSO at 0.4% DMSO in growth medium. Transfer 100 μl of the DMSO solution to the appropriate wells of cells. The final concentration of DMSO is 0.2%.
5. For the medium control wells, add 100 μl/well of growth medium to the appropriate wells of cells.
6. Return the plate to the incubator and incubate for 4 days.
   Day 5: Development of Assay
This part of assay is carried out on the bench.
1. Aspirate or pour off medium. Add 200 μl cold 10% TCA to each well to fix cells. Incubate plate for at least 60 min. at 4° C.
2. Discard TCA and rinse wells 5 times with water. Dry plates upside down on paper towels.
3. Stain cells with 100 μl/well 0.4% SRB for 10 min.
4. Pour off SRB and rinse wells 5 times with 1% acetic acid. Dry plates completely upside down on paper towels.
5. Solubilize dye with 100 μl/well 10 mM Tris base for 5-10 min. on shaker.
6. Read plates on Dynatech ELISA Plate Reader at 570 nm with reference at 630 nm.

### Assay 2: 3T3/EGF-R+TGF-a(T) Cell Growth SRB Assay

Materials and Reagents Same as for Assay 1.

Cell Line and Growth Medium:

3T3/EGF-R+TGF-a(T) (NIH 3T3 clone 7 cells expressing EGF-R and TGF-a, tumor-derived autocrine loop cells) 2% calf serum/DMEM+2 mM glutamine
   Protocol:
   Day 0: Cell Plating:
This part of assay is carried out in a laminar flow hood.
1. Trypsinize cells as usual. Transfer 100 μl of cell suspension to 10 ml of isotone. Count cells with the Coulter Counter.
2. Dilute cells in growth medium to 60,000 cells/ml. Transfer 100 μl of cells to each well in a 96-well flat bottom plate to give 6000 cells/well.
3. Use half of plate (4 rows) for each compound and quadruplicate wells for each compound concentration, a set of 4 wells for medium control and 4 wells for DMSO control.
4. Gently shake plates to allow for uniform attachment of the cells.
5. Incubate the plates at 37° C. in a 10% CO2 incubator.
Day 1: Addition of Compound: same as for Assay 1.
Day 5: Development of Assay: same as for Assay 1.

### Assay 3: 3T3/PDGF-βR/PDGF-BB(T) Cell Growth SRB Assay

Cell Line and Growth Medium:

3T3/PDGF-βR/PDGF-BB(T) (NIH 3T3 clone 7 cells expressing PDGFβ-receptor and PDGF-BB, from tumors resected from athymic mice) 2% calf serum/DMEM+2 mM glutamine
   Protocol:
   Day 0: Cell Plating:
This part of assay is carried out in a laminar flow hood.
1. Trypsinize cells as usual. Transfer 200 μl of cell suspension to 10 ml of isotone. Count cells on the Coulter Counter.
2. Dilute cells in growth medium to 60,000 cells/ml. Transfer 100 μl of cells to each well in a 96-well flat bottom plate to give 6000 cells/well.
3. Allow half of plate (4 rows) for each compound and quadruplicate wells for each compound concentration, a set of 4 wells for medium control and 4 wells for DMSO control.
4. Gently shake plates to allow for uniform attachment of the cells to the plate.
5. Incubate the plates at 37° C. in a 10% CO₂ incubator.
Day 1: Addition of Compound: same as for Assay 1.
Day 5: Development of Assay: same as for Assay 1.

### Assay 4: Human Smooth Muscle Cells (SMC) Growth SRB Assay

Materials and Reagents Same as for Assay 1:

Cell Line and Growth Medium:

Human Aortic Smooth Muscle cells (Clonetics)
Clonetics's Bullet Kit: Smooth Muscle Basal Medium (SmBM) which is modified MCDB 131 containing fetal bovine serum (5%), hFGF (2 ng/ml), hEGF (0.1 ng/ml), insulin (5.0 ug/ml), gentamicin (50 ug/ml) and amphotericin B (50 ng/ml)
   Protocol:
   Day 0: Cell plating:
This part of assay is carried out in a laminar flow hood.
1. Trypsinize cells as usual. Transfer 200 μl of cell suspension to 10 ml of isotone. Count cells on the Coulter Counter.
2. Dilute cells in growth medium to 20,000 cells/ml. Transfer 100 μl of cells to each well in a 96-well flat bottom plate to give 2000 cells/well.
3. Allow half of plate (4 rows) for each compound and quadruplicate wells for each compound concentration, a set of 4 wells for medium control and 4 wells for DMSO control.
4. Gently shake plates to allow for uniform attachment of the cells to the plate.
5. Incubate the plates at 37° C. in a 10% CO2 incubator.
Day 1: Addition of Compound: same as for Assay 1.
Day 5: Development of Assay: same as for Assay 1.

### 3T3 Cell Growth Assay Assay 1: PDGF-Induced BrdU Incorporation Assay

Materials and Reagents:

(1) PDGF: human PDGF B/B; 1276-956, Boehringer Mannheim, Germany
(2) BrdU Labeling Reagent: 10 mM, in PBS (pH7.4), Cat. No. 1 647 229, Boehringer Mannheim, Germany.
(3) FixDenat: fixation solution (ready to use), Cat. No. 1 647 229, Boehringer Mannheim, Germany.
(4) Anti-BrdU-POD: mouse monoclonal antibody conjugated with peroxidase, Cat. No. 1 647 229, Boehringer Mannheim, Germany.
(5) TMB Substrate Solution: tetramethylbenzidine (TMB), ready to use, Cat. No. 1 647 229, Boehringer Mannheim, Germany.
(6) PBS Washing Solution: 1× PBS, pH 7.4, made in house.
(7) Albumin, Bovine (BSA): fraction V powder; A-8551, Sigma Chemical Co., USA.
   Protocol
(1) 3T3 engineered cell line: 3T3/EGFRc7.
(2) Cells are seeded at 8000 cells/well in DMEM, 10% CS, 2 mM Gin in a 96 well plate. Cells are incubated overnight at 37° C. in 5% CO₂.
(3) After 24 hours, the cells are washed with PBS, and then are serum starved in serum free medium (0% CS DMEM with 0.1% BSA) for 24 hours.
(4) On day 3, ligand (PDGF=3.8 nM, prepared in DMEM with 0.1% BSA) and test compounds are added to the cells simultaneously. The negative control wells receive serum free DMEM with 0.1% BSA only; the positive control cells receive the ligand (PDGF) but no test compound. Test compounds are prepared in serum free DMEM with ligand in a 96 well plate, and serially diluted for 7 test concentrations.
(5) After 20 hours of ligand activation, diluted BrdU labeling reagent (1:100 in DMEM, 0.1% BSA) is added and the cells are incubated with BrdU (final concentration=10 μM) for 1.5 hours.
(6) After incubation with labeling reagent, the medium is removed by decanting and tapping the inverted plate on a paper towel. FixDenat solution is added (50 μl/well) and the plates are incubated at room temperature for 45 minutes on a plate shaker.
(7) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.
(8) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.
(9) The antibody conjugate is thoroughly removed by decanting and rinsing the wells 5 times with PBS, and the plate is dried by inverting and tapping on a paper towel.
(10) TMB substrate solution is added (100 μl/well) and incubated for 20 minutes at room temperature on a plate shaker until color development is sufficient for photometric detection.
(11) The absorbance of the samples is measured at 410 nm (in “dual wavelength” mode with a filter reading at 490 nm, as a reference wavelength) on a Dynatech ELISA plate reader.

### Assay 2: EGF-Induced BrdU Incorporation Assay

Materials and Reagents

(1) EGF: mouse EGF, 201; Toyobo,Co., Ltd. Japan
(2) BrdU Labeling Reagent: 10 mM, in PBS (pH7.4), Cat. No. 1 647 229, Boehringer Mannheim, Germany.
(3) FixDenat: fixation solution (ready to use), Cat. No. 1 647 229, Boehringer Mannheim, Germany.
(4) Anti-BrdU-POD: mouse monoclonal antibody conjugated with peroxidase, Cat. No. 1 647 229, Boehringer Mannheim, Germany.
(5) TMB Substrate Solution: tetramethylbenzidine (TMB), ready to use, Cat. No. 1 647 229, Boehringer Mannheim, Germany.
(6) PBS Washing Solution: 1× PBS, pH 7.4, made in house.
(7) Albumin, Bovine (BSA): fraction V powder; A-8551, Sigma Chemical Co., USA.
   Protocol
(1) 3T3 engineered cell line: 3T3/EGFRc7
(2) Cells are seeded at 8000 cells/well in 10% CS, 2 mM Gln in DMEM, in a 96 well plate. Cells are incubated overnight at 37° C. in 5% CO₂.
(3) After 24 hours, the cells are washed with PBS, and then are serum starved in serum free medium (0% CS DMEM with 0.1% BSA) for 24 hours.
(4) On day 3, ligand (EGF=2 nM, prepared in DMEM with 0.1% BSA) and test compounds are added to the cells simultaneously. The negative control wells receive serum free DMEM with 0.1% BSA only; the positive control cells receive the ligand (EGF) but no test compound. Test compounds are prepared in serum free DMEM with ligand in a 96 well plate, and serially diluted for 7 test concentrations.
5) After 20 hours of ligand activation, diluted BrdU labeling reagent (1:100 in DMEM, 0.1% BSA) is added and the cells are incubated with BrdU (final concentration=10 μM) for 1.5 hours.
6) After incubation with labeling reagent, the medium is removed by decanting and tapping the inverted plate on a paper towel. FixDenat solution is added (50 μl/well) and the plates are incubated at room temperature for 45 minutes on a plate shaker.
(7) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.
(8) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.
(9) The antibody conjugate is thoroughly removed by decanting and rinsing the wells 5 times with PBS, and the plate is dried by inverting and tapping on a paper towel.
(10) TMB substrate solution is added (100 μl/well) and incubated for 20 minutes at room temperature on a plate shaker until color development is sufficient for photometric detection.
(11) The absorbance of the samples is measured at 410 nm (in “dual wavelength” mode with a filter reading at 490 nm, as a reference wavelength) on a Dynatech ELISA plate reader.

### Assay 3: EGF-Induced Her2-Driven BrdU Incorporation

Materials and Reagents:

(1) EGF: mouse EGF, 201; Toyobo, Co., Ltd. Japan
(2) BrdU Labeling Reagent: 10 mM, in PBS (pH7.4), Cat. No. 1 647 229, Boehringer Mannheim, Germany.
(3) FixDenat: fixation solution (ready to use), Cat. No. 1 647 229, Boehringer Mannheim, Germany.
(4) Anti-BrdU-POD: mouse monoclonal antibody conjugated with peroxidase, Cat. No. 1 647 229,-Boehringer Mannheim, Germany.
(5) TMB Substrate Solution: tetramethylbenzidine (TMB), ready to use, Cat. No. 1 647 229, Boehringer Mannheim, Germany.
(6) PBS Washing Solution: 1× PBS, pH 7.4, made in house.
(7) Albumin, Bovine (BSA): fraction V powder; A-8551, Sigma Chemical Co., USA.
   Protocol:
(1) 3T3 engineered cell line: 3T3/EGFr/Her2/EGFr (EGFr with a Her2 kinase domain)
(2) Cells are seeded at 8000 cells/well in DMEM, 10% CS, 2 mM Gln in a 96-well plate. Cells are incubated overnight at 37° C. in 5% CO₂.
(3) After 24 hours, the cells are washed with PBS, and then are serum starved in serum free medium (0% CS DMEM with 0.1% BSA) for 24 hours.
(4) On day 3, ligand (EGF=2 nM, prepared in DMEM with 0.1% BSA) and test compounds are added to the cells simultaneously. The negative control wells receive serum free DMEM with 0.1% BSA only; the positive control cells receive the ligand (EGF) but no test compound. Test compounds are prepared in serum free DMEM with ligand in a 96 well plate, and serially diluted for 7 test concentrations.
(5) After 20 hours of ligand activation, diluted BrdU labeling reagent (1:100 in DMEM, 0.1% BSA) is added and the cells are incubated with BrdU (final concentration=10 μM) for 1.5 hours.
(6) After incubation with labeling reagent, the medium is removed by decanting and tapping the inverted plate on a paper towel. FixDenat solution is added (50 μl/well) and the plates are incubated at room temperature for 45 minutes on a plate shaker.
(7) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.
(8) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.
(9) The antibody conjugate is thoroughly removed by decanting and rinsing the wells 5 times with PBS, and the plate is dried by inverting and tapping on a paper towel.
(10) TMB substrate solution is added (100 μl/well) and incubated for 20 minutes at room temperature on a plate shaker until color development is sufficient for photometric detection.
(11) The absorbance of the samples is measured at 410 nm (in “dual wavelength” mode with a filter reading at 490 nm, as a reference wavelength) on a Dynatech ELISA plate reader.

### Assay 4: IGF1-Induced BrdU Incorporation Assay

Materials and Reagents:

(1) IGF1 Ligand: human, recombinant; G511, Promega Corp, USA.
(2) BrdU Labeling Reagent: 10 mM, in PBS (pH7.4), Cat. No. 1 647 229, Boehringer Mannheim, Germany.
(3) FixDenat: fixation solution (ready to use), Cat. No. 1 647 229, Boehringer Mannheim, Germany.
(4) Anti-BrdU-POD: mouse monoclonal antibody conjugated with peroxidase, Cat. No. 1 647 229, Boehringer Mannheim, Germany.
(5) TMB Substrate Solution: tetramethylbenzidine (TMB), ready to use, Cat. No. 1 647 229, Boehringer Mannheim, Germany.
(6) PBS Washing Solution: 1× PBS, pH 7.4, made in house.
(7) Albumin, Bovine (BSA): fraction V powder; A-8551, Sigma Chemical Co., USA.
   Protocol:
(1) 3T3 engineered cell line: 3T3/IGF1r.
(2) Cells are seeded at 8000 cells/well in DMEM, 10% CS, 2 mM Gln in a 96-well plate. Cells are incubated overnight at 37° C. in 5% CO₂.
(3) After 24 hours, the cells are washed with PBS, and then are serum starved in serum free medium (0% CS DMEM with 0.1% BSA) for 24 hours.
(4) on day 3, ligand (IGF1=3.3 nM, prepared in DMEM with 0.1% BSA) and test compounds are added to the cells simultaneously. The negative control wells receive serum free DMEM with 0.1% BSA only; the positive control cells receive the ligand (IGF1) but no test compound. Test compounds are prepared in serum free DMEM with ligand in a 96 well plate, and serially diluted for 7 test concentrations.
5) After 16 hours of ligand activation, diluted BrdU labeling reagent (1:100 in DMEM, 0.1% BSA) is added and the cells are incubated with BrdU (final concentration=10 AM) for 1.5 hours.
(6) After incubation with labeling reagent, the medium is removed by decanting and tapping the inverted plate on a paper towel. FixDenat solution is added (50 μl/well) and the plates are incubated at room temperature for 45 minutes on a plate shaker.
(7) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.
(8) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.
(9) The antibody conjugate is thoroughly removed by decanting and rinsing the wells 5 times with PBS, and the plate is dried by inverting and tapping on a paper towel.
(10) TMB substrate solution is added (100 μl/well) and incubated for 20 minutes at room temperature on a plate shaker until color development is sufficient for photometric detection.
(11) The absorbance of the samples are measured at 410 nm (in “dual wavelength” mode with a filter reading at 490 nm, as a reference wavelength) on a Dynatech ELISA plate reader.

### Assay 5: Insulin-Induced BrdU Incorporation Assay

Materials and Reagents:

(1) Insulin: crystalline, bovine, Zinc; 13007, Gibco BRL, USA.
(2) BrdU Labeling Reagent: 10 mM, in PBS (pH7.4), Cat. No. 1 647 229, Boehringer Mannheim, Germany.
(3) FixDenat: fixation solution (ready to use), Cat. No. 1 647 229, Boehringer Mannheim, Germany.
(4) Anti-BrdU-POD: mouse monoclonal antibody conjugated with peroxidase, Cat. No. 1 647 229, Boehringer Mannheim, Germany.
(5) TMB Substrate Solution: tetramethylbenzidine (TMB), ready to use, Cat. No. 1 647 229, Boehringer Mannheim, Germany.
(6) PBS Washing Solution: 1× PBS, pH 7.4, made in house.
(7) Albumin, Bovine (BSA): fraction V powder; A-8551, Sigma Chemical Co., USA.
   Protocol:
(1) 3T3 engineered cell line: H25
(2) Cells are seeded at 8000 cells/well in DMEM, 10% CS, 2 mM Gin in a 96 well plate. Cells are incubated overnight at 37° C. in 5% CO₂.
(3) After 24 hours, the cells are washed with PBS, and then are serum starved in serum free medium (0% CS DMEM with 0.1% BSA) for 24 hours.
(4) On day 3, ligand (Insulin=10 nM, prepared in DMEM with 0.1% BSA) and test compounds are added to the cells simultaneously. The negative control wells receive serum free DMEM with 0.1% BSA only; the positive control cells receive the ligand (Insulin) but no test compound. Test compounds are prepared in serum free DMEM with ligand in a 96 well plate, and serially diluted for 7 test concentrations.
(5) After 16 hours of ligand activation, diluted BrdU labeling reagent (1:100 in DMEM, 0.1% BSA) is added and the cells are incubated with BrdU (final concentration=10 μM) for 1.5 hours.
(6) After incubation with labeling reagent, the medium is removed by decanting and tapping the inverted plate on a paper towel. FixDenat solution is added (50 μl/well) and the plates are incubated at room temperature for 45 minutes on a plate shaker.
(7) The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.
(8) The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.
(9) The antibody conjugate is thoroughly removed by decanting and rinsing the wells 5 times with PBS, and the plate is dried by inverting and tapping on a paper towel.
(10) TMB substrate solution is added (100 μl/well) and incubated for 20 minutes at room temperature on a plate shaker until color development is sufficient for photometric detection.
(11) The absorbance of the samples are measured at 410 nm (in “dual wavelength” mode with a filter reading at 490 nm, as a reference wavelength) on a Dynatech ELISA plate reader.

### HUV-EC-C Assay

The following protocol may also be used to measure the composition's activity:
Day 0
1. Wash and trypsinize HUV-EC-C cells (human umbilical vein endothelial cells, (American Type Culture Collection; catalogue no.1730 CRL). Wash with Dulbecco's phosphate-buffered saline (D-PBS; obtained from Gibco BRL; catalogue no. 14190-029) 2 times at about 1 ml/10 cm.sup.2 of tissue culture flask. Trypsinize with 0.05% trypsin-EDTA in non-enzymatic cell dissociation solution (Sigma Chemical Company; catalogue no. C-1544). The 0.05% trypsin was made by diluting 0.25% trypsin/1 mM EDTA (Gibco; catalogue no. 25200-049) in the cell dissociation solution. Trypsinize with about 1 ml/25-30 cm.sup.2 of tissue culture flask for about 5 minutes at 37° C. After cells have detached from the flask, add an equal volume of assay medium and transfer to a 50 ml sterile centrifuge tube (Fisher Scientific; catalogue no. 05-539-6).
2. Wash the cells with about 35 ml assay medium in the 50 ml sterile centrifuge tube by adding the assay medium, centrifuge for 10 minutes at approximately 200×g, aspirate the supernatant, and resuspend with 35 ml D-PBS. Repeat the wash two more times with D-PBS, resuspend the cells in about 1 ml assay medium/15 cm² of tissue culture flask. Assay medium consists of F12K medium (Gibco BRL; catalogue no. 21127-014)+0.5% heat-inactivated fetal bovine serum. Count the cells with a Coulter Counter.RTM.v Coulter Electronics, Inc.) and add assay medium to the cells to obtain a concentration of 0.8-1.0×10⁵ cells/ml.
3. Add cells to 96-well flat-bottom plates at 100 μl/well or 0.8-1.0.times.10.sup.4 cells/well; incubate about 24 h at 37° C., 5% CO₂.
   Day 1
1. Make up two-fold drug titrations in separate 96-well plates, generally 50 μM on down to 0 μM. Use the same assay medium as mentioned in day 0, step 2 above. Titrations are made by adding 90 μl/well of drug at 200 μM (4× the final well concentration) to the top well of a particular plate column. Since the stock drug concentration is usually 20 mM in DMSO, the 200 μM drug concentration contains 2% DMSO. Therefore, diluent made up to 2% DMSO in assay medium (F12K+0.5% fetal bovine serum) is used as diluent for the drug titrations in order to dilute the drug but keep the DMSO concentration constant. Add this diluent to the remaining wells in the column at 60 μl/well. Take 60 μl from the 120 μl of 200 μM drug dilution in the top well of the column and mix with the 60 μl in the second well of the column. Take 60 μl from this well and mix with the 60 μl in the third well of the column, and so on until two-fold titrations are completed. When the next-to-the-last well is mixed, take 60 μl of the 120 μl in this well and discard it. Leave the last well with 60 μl of DMSO/media diluent as a non-drug-containing control. Make 9 columns of titrated drug, enough for triplicate wells each for 1) VEGF (obtained from Pepro Tech Inc., catalogue no. 100-200, 2) endothelial cell growth factor (ECGF) (also known as acidic fibroblast growth factor, or aFGF) (obtained from Boehringer Mannheim Biochemica, catalogue no. 1439 600), and assay media control. ECGF comes as a preparation with sodium heparin.
2. Transfer 50 μl/well of the drug dilutions to the 96-well assay plates containing the 0.8-1.0×10⁴ cells/100 μl/well of the HUV-EC-C cells from day 0 and 20 incubate .about.2 h at 37° C., 5% CO₂.
3. In triplicate, add 50 μl/well of 80 ng/ml VEGF, 20 ng/ml ECGF, or media control to each drug condition. As with the drugs, the growth factor concentrations are 4× the desired final concentration. Use the assay media from day 0 step 2 to make the concentrations of growth factors. Incubate approximately 24 hours at 37° C., 5% CO₂. Each well will have 50 μl drug dilution, 50 μl growth factor or media, and 100 ul cells,=200 ul/well total. Thus the 4× concentrations of drugs and growth factors become 1× once everything has been added to the wells.
   Day 2
1. Add ³H-thymidine (Amersham; catalogue no. TRK-686) at 1 μCi/well (10 μl/well of 100 μCi/ml solution made up in RPMI media+10% heat-inactivated fetal bovine serum) and incubate about 24 h at 37° C., 5% CO₂.
Note: ³H-thymidine is made up in RPMI media because all of the other applications for which we use the ³H-thymidine involve experiments done in RPMI. The media difference at this step is probably not significant. RPMI was obtained from Gibco BRL, catalogue no. 11875-051.
   Day 3
1. Freeze plates overnight at −20° C.
   Day 4
1. Thaw plates and harvest with a 96-well plate harvester (Tomtec Harvester 96.RTM.) onto filter mats (Wallac; catalogue no. 1205-401); read counts on a Wallac Betaplate™ liquid scintillation counter.

### PDGF-R Cellular Assay

The PDGF cellular kinase assay was carried out as follows: cells are lysed in 0.2M Hepes, 0.15M NaCl, 10% V/V glycerol, 0.04% Triton X-100, 5 mM EDTA, 5 mM sodium vanadate and 2 mM Na+pyrophosphate; cell lysates are then added to an ELISA plate coated with an anti-PDGF receptor antibody (Genzyme); ELISA plates are coated at 0.5 μg of antibody/well in 150 μl of PBS for 18 hours at 4° C. prior to the addition of the lysate; the lysate is incubated in the coated plates for 1 hour and then washed four times in TBST (35 mM Tris-HCl pH 7.0, 0.15M NaCl, 0.1% Triton X100); anti-phosphotyrosine antibody (100 μl in PBS) is added and the mixture is incubated for 30 minutes at room temperature; the wells were then washed four times in TBST, a secondary antibody conjugated to POD (TAGO) is added to each well, and the treated wells are incubated for 30 minutes at room temperature; the wells are then washed four times in TBST, ABTS/H₂ O₂ solution is added to each well and the wells are incubated for two minutes; absorbance is then measured at 410 nm.

### Experimental Results of Cell Growth Assay

Results for various compounds obtained from the above-described assays are set forth in the Tables that follow:

TABLE 2
Mitogenesis in Endothelial Cells
[3H] Thymidine Incorporation

| | HUV-EC | Assay |
|---|---|---|
| COMPOUND | VEGF (μM) | a-FGF (μM) |
| SU4312 | 1.1 | 153.8 |
| SU4314 | 0.2 | 6.0 |
| SU4793 | 6.6 | 3.4 |
| SU4794 | 4.8 | 35.7 |
| SU4796 | 30.7 | 35.8 |
| SU4798 | 43.2 | |
| SU4799 | 19.9 | |
| SU4932 | 2.5 | 45.2 |
| SU4942 | 1.6 | 4.6 |
| SU4944 | 14.8 | |
| SU4949 | 3.4 | 3.7 |
| SU4952 | 25.6 | 19.3 |
| SU4956 | 8.0 | 13.0 |
| SU4967 | 34.3 | 16.3 |
| SU4972 | 1.0 | 1.4 |
| SU4979 | 4.4 | 4.9 |
| SU4981 | 0.6 | |
| SU4982 | 46.1 | 27.3 |
| SU4984 | 0.8 | 25.8 |
| SU5201 | 2.5 | 2.3 |
| SU5204 | 2.3 | 0.7 |
| SU5205 | 5.1 | 11.8 |
| SU5208 | 2.9 | 130 |
| SU5217 | 9.6 | 10.5 |
| SU5218 | 2.4 | 2.7 |
| SU5401 | 2.2 | |
| SU5402 | <0.8 | 2.0 |
| SU5404 | <0.8 | 31.1 |
| SU5405 | 0.9 | 0.6 |
| SU5406 | <0.8 | |
| SU5407 | 39.8 | 35.5 |
| SU5408 | <0.8 | 22.7 |
| SU5409 | 26.0 | |
| SU5416 | <0.8 | |
| SU5418 | 13.6 | 40 |
| SU5419 | 0.7 | |
| SU5421 | 11.4 | |
| SU5424 | 2.5 | |
| SU5427 | 5.7 | |
| SU5429 | 27.6 | |
| SU5432 | 0.16 | 0.14 |
| SU5438 | 39.8 | 33.0 |
| SU5451 | 1.2 | 30.0 |
| SU5454 | 3.8 | 3.4 |
| SU5455 | 20 | 20 |
| SU5461 | <0.07 | <0.07 |
| SU5462 | 0.5 | 0.8 |
| SU5463 | 0.14 | 7.9 |
| SU5464 | 3.8 | 12.9 |
| SU5466 | 1.3 | 3.2 |
| SU5468 | 0.54 | 8.7 |
| SU5472 | 2.0 | 5.0 |
| SU5473 | 1.2 | 14.1 |
| SU5477 | 0.05 | 37.8 |
| SU5480 | 1.2 | 3.8 |

TABLE 3
Mitogenesis in 3T3/EGFR Cells
BrdU Incorporation

| | PDGFR | FGFR | EGFR |
|---|---|---|---|
| | PDGF Ligand | FGF Ligand | EGF Ligand |
| CMPD. | IC50 (μM) | IC50 (μM) | IC50 (μM) |
| SU4312 | 75 | | |
| SU4313 | 6 | 5.5 | 5.5 |
| SU4314 | 2.5 | | |
| SU4967 | 9 | 4.9 | 60 |
| SU4981 | 3 | 10 | 20 |
| SU5402 | 50 | 40 | |
| SU5404 | 3 | 25 | |
| SU5406 | 5.2 | | |
| SU5407 | 7.5 | 70 | 100 |
| SU5416 | 2.8 | 70 | |
| SU5451 | 30 | 16 | |
| SU5463 | | | 23 |
| SU5464 | 70 | 60 | 95 |
| SU5465 | 40 | 25 | 50 |
| SU5466 | 18 | 15 | 17 |
| SU5468 | 8 | | |
| SU5469 | 4 | 15 | 28 |
| SU5473 | 4 | 50 | 54 |
| SU5475 | 6.5 | 9 | 48 |

TABLE 4
Cell Growth Assay on Various Cell Lines
SRB Readout

| | 3T3/E/H+ | 3T3/EGFR+ | 3T3/PDGFR+ | |
|---|---|---|---|---|
| | TGF-a(T) | TGF-a(T) | PDGF (T) | SMC |
| | IC50 (μM) | IC50 (μM) | IC50 (μM | IC50 (μM) |
| SU4312 | 36 | | | |
| SU4313 | 32 | 10.7 | | 8.8 |
| SU4314 | 78 | | 10 | |
| SU4984 | | | 22.2 | |

3T3/E/H+TGF-α(T): NIH 3T3 cells expressing EGFR/HER2 chimera and TGF-α, tumor-derived 3T3/EGFR+TGF-α(T): NIH 3T3 cells expressing EGFR and TGF-α, tumor-derived 3T3/PDGFR+PDGF(T): NIH 3T3 cells expressing PDGF-βR and PDGF-ββ, tumor-derived SMC: human smooth muscle cells from Clonetics

### Measurement of Cell Toxicity

Therapeutic compounds should be more potent in inhibiting receptor tyrosine kinase activity than in exerting a cytotoxic effect. A measure of the effectiveness and cell toxicity of a compound can be obtained by determining the therapeutic index: IC₅₀/LD₅₁. IC₅₀, the dose required to achieve 50% inhibition, can be measured using standard techniques such as those described herein. LD₅₀, the dosage which results in 50% toxicity, can also be measured by standard techniques (Mossman, 1983, J. Immunol. Methods, 65:55-63), by measuring the amount of LDH released (Korzeniewski and Callewaert, 1983, J. Immunol. Methods 64:313; Decker and Lohmann-Matthes, 1988, J. Immunol. Methods 115:61), or by measuring the lethal dose in animal models. Compounds with a large therapeutic index are preferred. The therapeutic index should be greater than 2, preferably at least 10, more preferably at least 50.

### In Vivo Animal Models Xenograft Animal Models

The ability of human tumors to grow as xenografts in athymic mice (e.g., Balb/c, nu/nu) provides a useful in vivo model for studying the biological response to therapies for human tumors. Since the first successful xenotransplantation of human tumors into athymic mice, (Rygaard and Povlsen, 1969, Acta Pathol. Microbial. Scand. 77:758-760), many different human tumor cell lines (e.g., mammary, lung, genitourinary, gastrointestinal, head and neck, glioblastoma, bone, and malignant melanomas) have been transplanted and successfully grown in nude mice. Human mammary tumor cell lines, including MCF-7, ZR75-I, and MDA-MB-231, have been established as subcutaneous xenografts in nude mice (Warri et al., 1991, Int. J. Cancer 49:616-623; Ozzello and Sordat, 1980, Eur. J. Cancer 16:553-559; Osborne et al., 1985, Cancer Res. 45:584-590; Seibert et al., 1983, Cancer Res. 43:2223-2239).

### Assay 1: HER2/Xenograft Animal Model

To study the effect of anti-tumor drug candidates on HER2 expressing tumors, the tumor cells should be able to grow in the absence of supplemental estrogen. Many mammary cell lines are dependent on estrogen for in vivo growth in nude mice (Osborne et al., supra), however, exogenous estrogen suppresses HER2 expression in nude mice (Warri et al., supra, Dati et al., 1990, Oncogene 5:1001-1006). For example, in the presence of estrogen, MCF-7, ZR-75-1, and T47D cells grow well in vivo, but express very low levels of HER2 (Warri et al., supra, Dati et al., supra).

The Following Type of Xenograft Protocol Can be Used:

1) implant tumor cells (subcutaneously) into the hindflank of five- to six-week-old female Balbfc nu/nu athymic mice;
2) administer the anti-tumor compound;
3) measure tumor growth by measuring tumor volume.
   The tumors can also be analyzed for the presence of a receptor, such as HER2, EGF or PDGF, by Western and immunohistochemical analyses. Using techniques known in the art, one skilled in the art can vary the above procedures, for example through the use of different treatment regimes.

### Assay 2: FLK-1/Xenograft Model

The ability of the compounds of the present invention to inhibit ovarian, melanoma, prostate, lung and mammary tumor cell lines established as SC xenografts was examined. These studies were conducted using doses ranging from 1 to 75 mg/kg/day.

Materials And Methods. The tumor cells were implanted subcutaneously into the indicated strains of mice. Treatment was initiated on day 1 post implantation unless otherwise indicated (e.g. treatment of the SCID mouse related to the A375 melanoma cell line began on Day 9). Eight (8) to sixteen (16) mice comprised each test group.

Specifically:

Animals. Female athymic mice (BALB/c, nu/nu), BALB/c mice, Wistar rats and Fisher 344 rats were obtained from Simonsen Laboratories (Gilroy, Calif.). Female A/I mice were obtained from Jackson Laboratory (Bar Harbor, Me.). DA rats were obtained from B&K Universal, Inc. (Fremont, Calif.). Athymic R/Nu rats, DBA/2N mice, and BALB/c mice were obtained from Harlan Sprague Dawley (Indianapolis, Ind.). Female C57BL/6 mice were obtained from Taconic (Germantown, N.Y.). All animals were maintained under clean-room conditions in Micro-isolator cages with Alpha-dri bedding. They received sterile rodent chow and water ad libitum.

All procedures were conducted in accordance with the NIH Guide for the Care and Use Of Laboratory Animals.

Subcutaneous Xenograft Model. Cell lines were grown in appropriate medium as described. Cells were harvested at or near confluency with 0.05% Trypsin-EDTA and pelleted at 450.times.g for 10 min. Pellets were resuspended in sterile PBS or media (without FBS) to a suitable concentration indicated in the Figure legends and the cells were implanted into the hindflank of mice. Tumor growth was measured over 3 to 6 weeks using venier calipers and tumor volumes were calculated as a product of length x width x height unless otherwise indicated. P values were calculated using the Students' t-test.

Different concentrations of a compound in 50-100 μl excipient (dimethylsulfoxide, PBTE, PBTE6C:D5W, or PBTE:D5W) were delivered by IP injection.

Intracerebral Xenograft Model. For the mouse IC model, rat C6 glioma cells were harvested and suspended in sterile PBS at a concentration of 2.5×10⁷ cells/ml and placed on ice. Cells were implanted into BALB/c, nu/nu mice in the following manner: the frontoparietal scalps of mice were shaved with animal clippers if necessary before swabbing with 70% ethanol. Animals were anesthetized with isofluorane and the needle was inserted through the skull into the left hemisphere of the brain. Cells were dispensed from Hamilton Gas-tight Syringes using 30 ga ½ inch needles fitted with sleeves that allowed only a 3 mm penetration. A repeater dispenser was used for accurate delivery of 4 μl of cell suspension. Animals were monitored daily for well-being and were sacrificed when they had a weight loss of about 40% and/or showed neurological symptoms. For the rat IC model, rats (Wistar, Sprague Dawley, Fisher 344, or athymic R/Nu, approximately 200-400 g (some 3-400 g)) were anesthetized by an IP injection of 100 mg/kg Ketaset (ketamine hydrochloride; Aveco, Fort Dodge, Iowa) and 5 mg/kg Rompun (xylazine, 2% solution; Bayer, Germany). After onset of anesthesia, the scalp was shaved and the animal was oriented in a stereotaxic apparatus (Stoelting, Wood Dale, Ill.). The skin at the incision site was cleaned 3 times with alternating swabs of 70% ethanol and 10% Povidone-Iodine. A median 1.0-1.5 cm incision was made in the scalp using a sterile surgical blade. The skin was detached slightly and pulled to the sides to expose the sutures on the skull surface. A dental drill (Stoelting, Wood Dale, Ill.) was used to make a small (1-2 mm diameter) burrhole in the skull approximately 1 mm anterior and 2 mm lateral to the bregma. The cell suspension was drawn into a 50 μl Hamilton syringe fitted with a 23 or 25 g a standard bevel needle. The syringe was oriented in the burrhole at the level of the arachnoidea and lowered until the tip of the needle was 3 mm deep into the brain structure, where the cell suspension was slowly injected. After cells were injected, the needle was left in the burrhole for 1-2 minutes to allow for complete delivery of the cells. The skull was cleaned and the skin was closed with 2 to 3 sutures. Animals were observed for recovery from surgery and anesthesia. Throughout the experiment, animals were observed at least twice each day for development of symptoms associated with progression of intracerebral tumor. Animals displaying advanced symptoms (leaning, loss of balance, dehydration, loss of appetite, loss of coordination, cessation of grooming activities, and/or significant weight loss) were humanely sacrificed and the organs and tissues of interest were resected.

Intraperitoneal Model. Cell lines were grown in the appropriate media. Cells were harvested and washed in sterile PBS or medium without FBS, resuspended to a suitable concentration, and injected into the IP cavity of mice of the appropriate strain. Mice were observed daily for the occurrence of ascites formation. Individual animals were sacrificed when they presented with a weight gain of 40%, or when the IP tumor burden began to cause undue stress and pain to the animal.

### In Vivo VEGF Pellet Model

In the following example, the Pellet Model was used to test a compound's activity against the FLK-1 receptor and against disorders associated with the formation of blood vessels. In this model, VEGF is packaged into a time-release pellet and implanted subcutaneously on the abdomen of nude mice to induce a ‘reddening’ response and subsequent swelling around the pellet. Potential FLK-1 inhibitors may then be implanted in methylcellulose near the VEGF pellet to determine whether such inhibitor may be used to inhibit the “reddening” response and subsequent swelling.

Materials And Methods. The Following Materials were Used:

1) VEGF-human recombinant lyophilized product is commercially available and may be obtained from Peprotech, Inc., Princeton Business Park, G2; P.O. box 275, Rocky Hill, N.J. 08553.
2) VEGF packaged into 21 day release pellets were obtained from Innovative Research of America (Innovative Research of America, 3361 Executive Parkway, P.O. Box 2746, Toledo, Ohio 43606), using patented matrix driven delivery system. Pellets were packaged at 0.20, 0.21, or 2.1 μg VEGF/pellet. These doses approximate 10 and 100 ng/day release of VEGF.
3) Methylcellulose
4) Water (sterile)
5) Methanol
6) Appropriate drugs/inhibitors
7)10 cm culture plates
8) parafilm
   The Following Protocol was Then Followed to Conduct the VEGF Pellet Model:
1) VEGF, purchased from Peprotech, was sent to Innovative Research for Custom Pellet preparation;
2) Methylcellulose prepared at 1.5% (w/v) in sterile water;
3) Drugs solubilized in methanol (usual concentration range=10 to 20 mg/ml);
4) Place sterile parafilm in sterile 10 cm plates;
5) 150 μl of drug in methanol added to 1.35 ml of 1.5% methylcellulose and mixed/vortexed thoroughly;
6) 25 μl aliquots of homogenate placed on parafilm and dried into discs;
7) Mice (6-10 wk. Balb/C athymic nu/nu, female) were anesthetized via isoflurane inhalation;
8) VEGF pellets and methylcellulose discs were implanted subcutaneously on the abdomen; and
9) Mice were scored at 24 hours and 48 hours for reddening and swelling response.
   The Specific Experimental Design Used in this Example Was:
N=4 animals/group
Controls: VEGF pellet+drug placebo
VEGF placebo+drug pellet
Experimental Results. The compounds of the present invention are expected to demonstrate activity according to this assay.

### Mammary Fat Pad Model

Because of the established role played by many of the RTKs, e.g., the HER2 receptor, in breast cancer, the mammary fat pad model is particularly useful for measuring the efficacy of compounds which inhibit such RTKs. By implanting tumor cells directly into the location of interest, in situ models more accurately reflect the biology of tumor development than do subcutaneous models. Human mammary cell lines, including MCF-7, have been grown in the mammary fat pad of athymic mice. Shafie and Grantham, 1981, Natl. Cancer Instit. 67:51-56; Gottardis et al., 1988, J. Steroid Biochem. 30:311-314. More specifically, the is following procedure can be used to measure the inhibitory effect of a compound on the HER2 receptor:
1) Implant, at various concentrations, MDA-MB-231 and MCF-7 cells transfected with HER-2 into the axillary mammary fat pads of female athymic mice;
2) Administer the compound; and
3) Measure the tumor growth at various time points.
   The tumors can also be analyzed for the presence of a receptor such as HER2, by Western and immunohistochemical analyses. Using techniques known in the art, one skilled in the art can vary the above procedures, for example through the use of different treatment regimes.

### Tumor Invasion Model

The following tumor invasion model has been developed and may be used for the evaluation of therapeutic value and efficacy of compositions of interest.

Procedure

8 week old nude mice (female) (Simonsen Inc.) were used as experimental animals. Implantation of tumor cells was performed in a laminar flow hood. For anesthesia, Xylazine/Ketamine Cocktail (100 mg/kg ketamine and 5 mg/kg) are administered intraperitoneally. A midline incision is done to expose the abdominal cavity (approximately 1.5 cm in length) to inject 10⁷ tumor cells in a volume of 100 μl medium. The cells are injected either into the duodenal lobe of the pancreas or under the serosa of the colon. The peritoneum and muscles are closed with a 6-0 silk continuous suture and the skin was closed by using would clips. Animals were observed daily.

### Analysis

After 2-6 weeks, depending on gross observations of the animals, the mice are sacrificed, and the local tumor metastases, to various organs (lung, liver, brain, stomach, spleen, heart, muscle) are excised and analyzed (measurements of tumor size, grade of invasion, immunochemistry, and in situ hybridization).

### Results

Results for various compounds obtained from the above-described in vivo assays are set forth at Table 5, below:

TABLE 5
In Vivo Data

| | EpH4-VEGF |
|---|---|
| COMPOUND | % inhibition @ mg/kg |
| SU4312 | 56% @ 75 |
| | 50% @ 75 |
| | 63% @ 50 |
| SU4932 | 42% @ 75 |
| | — |
| | 42% @ 50/50 |
| SU4942 | 46% @ 50 |
| | 47% @ 25 |
| SU5416 | 50% @ 25 |
| | — |
| | 57% @ 37.5/37.5 |
| SU5424 | 45% @ 50 |
| | — |
| | 65% @ 50 |
| SU5427 | 47% @ 50 |
| | — |
| | 65% @ 50 |

The present invention is not to be limited in scope by the exemplified embodiments which are intended as illustrations of single aspects of the invention. Indeed, various modifications of the invention in addition to those described herein will become apparent to those skilled in the art from the foregoing description. Such modifications are intended to fall within the scope of the appended claims.

All references cited herein are hereby incorporated by reference in their entirety.

### Example 3

### Combination of Celecoxib and SU-5416 Results in Tumor Inhibition in HN1483 Tumor Model

Human tumor xenograft nude mice (HN1483) were used to investigate the tumor inhibitive effects of combinations of Celecoxib and SU-5416. Human tumor xenograft nude mouse models of head and neck squamous cell carcinoma (1483 cell line) express COX-2 in the tumor cells and in the vascularture, similar to human epithelial cancers. Matrigel (30%) is mixed with cell suspension which results in a 100% occurrence of tumor growth. In this way, the HN1483 mice model human epithelial cancers expressing cyclooxgenase-2 (COX-2) in the tumor cells and in the vasculature and are a good model to correlate efficacy of anti-cancer drugs including COX-2 inhibitors to efficacy in humans.

### HN1483 Protocol

### Materials and Methods:

Cell Culture:

1483 human head and neck squamous cell carcinoma (HNSCC) cells are stored in frozen vials containing 3×10⁶ cells, 90% fetal bovine serum (FBS) and 10% dimethyl sulfoxide (DMSO). Take a frozen vial and quickly thaw at 37° C. and placed in a T-162 cm² (Corning) flask containing D-MEM/F12 media (GibcoBRL) with 15 mM Hepes buffer, L-glutamine, pyridoxine hydrochloride and 10% FBS. Cells are grown in a incubator with 5% CO₂ and temperature at 37° C. Media is change every other day and cells are passed when at 80-90% confluence. For passing of cells, wash flask with 10 ml of phosphate buffered saline (PBS), aspirate off and add 2 ml of trypsin/EDTA (0.25%/1 mM, GibcoBRL) place back in incubator, after 5 min cells will detach. Add 8 ml of above media to flask rinse and transfer to a sterile 50 ml centrifuge tube. Add 30 ml more of media and mix and count cells using a hemacytometer, plate out cells in a T-162 cm² containing 3-4×10⁶ cells.

1483 Animal Model:

Change media 24 hours before harvest of 1483 cells before injection in to nude mice. Trypsinize 1483 cells as described above in cell culture section. Count cells and determine number of cells. Centrifuge cells down at 1000 rpm for 5 minutes at room temperature. Resuspened cell pellets and pool them (if multiple 50 ml centrifuge tubes) into one 50 ml centrifuge tube with Hank's buffered saline solution (HBSS, GibcoBRL) and centrifuge as before. Extra cells may be obtained, if preferred. Prepare the cells for injecting into mice. 1483 cells are injected at 1×10⁶ cells in 0.03 ml/mouse. 100 mice×0.03 ml=3 ml total volume. Cells are injected with 30% Matrigel (Collaborative Biomedical Products) and 70% HBSS. Resuspend pooled pellet with 2.1 ml (70%) of cold HBSS then add 0.9 ml (30%) of thawed liquefied cold Matrigel and mix well on ice. Keep this cell prep on ice at all times prior to injecting into mice.

Male nude mice age 4-6 weeks old were used in the studies (Harlen). Mice are anesthetized using CO₂/O₂ gas and mice are injected in the middle of the right hind paw using a 0.5 cc tuberculin syringe (Beckerson & Dickerson). Mice are weighed for body weight on day of injection (Day 0) for baseline weight for start of study. Starting on day 7 mice are weighed and right hind paw are measure for paw tumor volume using a plethysmometer (Stoelting Co.). The plethysmometer is a machine that measure paw volume by water displacement. A few left non-injected paws are measured and averaged for a background measurement to subtract from the right tumor bearing paw. Mice are weighed and measured throughout the study on days 7, 10, 14, 17, 21, 24 and 28. Animals can be started on compound treatment on day 0 (prophylactic) or once there is a established tumor around day 7 (therapeutic). Around day 30 vehicle (control) mice will have large tumors (˜1.0-1.5 ml) and start to lose weight, at this time, vehicle animals may be terminated.

### Protocol for Treatment of HN1483 Mice with Celecoxib, SU-5416 and Combinations Thereof

Outcomes:

1.) Tumor growth, inhibition
2.) Body weight as health assessment

Cells will be injected into the right paws at a concentration of 1×10⁶ cells/paw in HBSS with 30% Matrigel.

| Paw | Groupn | drug | dose (mg/kg/day) | ppm |
|---|---|---|---|---|
| 1 | 12 | Vehicle | | |
| 2 | 8 | SU-5416 | 25 | |
| 3 | 8 | SU-5416/Celecoxib | 25 | 40 |
| 4 | 8 | SU-5416/Celecoxib | 25 | 160 |
| 5 | 8 | SU-5416 | 50 | |
| 6 | 8 | SU-5416/Celecoxib | 50 | 40 |
| 7 | 8 | SU-5416/Celecoxib | 50 | 160 |
| 8 | 8 | Celecoxib | | 40 |
| 9 | 8 | Celecoxib | | 160 |

SU-5416 was given s.c. daily and Celecoxib will be administered half in the meal and half by gavage at 11:00 am. Animals were ear notched and housed in polycarbs with bedding, 4 animals/polycarb. Animals were placed on normal Chow meal upon arrival and placed on test compound in Chow meal when tumors are 100-200 ul in size and continued on compound meal throughout study. Body weight was measured twice weekly. Tumor Volume was measured twice a week using a plethysmometer.

### Data Regarding Tumor Volume of the Treated Mice

Table 6 illustrates the raw data showing tumor volume measurements of the treated mice.

### Data Regarding Weights of Treated HN1483 Mice

Data regarding the weights of the HN1483 mice treated with Celecoxib, SU-5416 and combinations thereof are reproduced in Table 7.

TABLE 6
Raw Data Showing Tumor Volume of Treated HN1483 Mice

Mice Weighed day of injection
Paw volume

| | | | | | Day 31 | | | Day 31 | | | Day 24 | | |
|---|---|---|---|---|---|---|---|---|---|---|---|---|---|
| As- | | | | | Apr. 2, | | | Mar. 30, | | | Mar. 23, | | |
| signed | | | original | | 2001 | BKG. | Tumor | 2001 | BKG. | Tumor | 2001 | BKG. | Tumor |
| Group | | Cage # | cage | | paw | 0.10 | vol. | paw | 0.10 | vol. | paw | 0.10 | vol. |
| 1 | Vehicle | 1a | 10 | 1 | 0.20 | | 0.19 | 0.28 | | 0.18 | 0.24 | | 0.14 |
| | | | | 2 | 0.65 | | 0.55 | 0.54 | | 0.44 | 0.32 | | 0.22 |
| | | | | 3 | 0.26 | | 0.16 | 0.22 | | 0.12 | 0.2 | | 0.10 |
| | | | | 4 | 0.28 | | 0.16 | 0.27 | | 0.17 | 0.23 | | 0.13 |
| | | 1b | 11 | 5 | 1.32 | | 1.22 | 1.08 | | 0.95 | 0.56 | | 0.46 |
| | | | | 6 | 1.7 | | 1.60 | 1.41 | | 1.31 | 0.64 | | 0.54 |
| | | | | 7 | 0.49 | | 0.39 | 0.42 | | 0.32 | 0.27 | | 0.17 |
| | | | | 8 | 1.49 | | 1.39 | 1.11 | | 1.01 | 0.47 | | 0.37 |
| | | | 20 | 9 | 1.4 | | 1.30 | 1.24 | | 1.14 | 0.56 | | 0.46 |
| | | | | 10 | 1.44 | | 1.34 | 1.17 | | 1.07 | 0.71 | | 0.61 |
| | | | | 11 | 0.65 | | 0.55 | 0.46 | | 0.38 | 0.3 | | 0.20 |
| | | | | 12 | 0.61 | | 0.51 | 0.49 | | 0.39 | 0.3 | | 0.20 |
| | | Average | | | | | 0.78 | | | 0.83 | | | 0.30 |
| | | BEM | | | | | 0.187 | | | 0.126 | | | 0.081 |
| | | STDEV | | | | | 0.54 | | | 0.44 | | | 0.16 |
| 2 | SU5416 | 2a | 2 | 13 | 0.82 | | 0.72 | 0.84 | | 0.74 | 0.59 | | 0.49 |
| | 25 mg/kg/day s.c. | | | 14 | 0.79 | | 0.69 | 0.5 | | 0.70 | 0.8 | | 0.50 |
| | | | | 15 | 0.57 | 0.1 | 0.47 | 0.71 | | 0.61 | 0.37 | | 0.27 |
| | | | | 16 | 0.6 | | 0.50 | 0.23 | | 0.13 | 0.4 | | 0.30 |
| | | 2b | 3 | 17 | 0.66 | | 0.56 | 0.73 | | 0.63 | 0.49 | | 0.30 |
| | | | | 18 | 0.98 | | 0.68 | 0.81 | | 0.71 | 0.65 | | 0.55 |
| | | | | 19 | 0.75 | | 0.65 | 0.54 | | 0.44 | 0.61 | | 0.51 |
| | | | | 20 | 0.21 | | 0.11 | 0.56 | | 0.46 | 0.22 | | 0.12 |
| | | Average | | | | | 0.87 | | | 0.56 | | | 0.39 |
| | | SEM | | | | | 0.081 | | | 0.072 | | | 0.083 |
| | | STDEV | | | | | 0.23 | | | 0.20 | | | 0.19 |
| 3 | SU-5416/25 mgkd | 3a | 4 | 21 | 0.17 | | 0.07 | 0.22 | | 0.12 | 0.27 | | 0.17 |
| | Calecoxib/40 ppm | | | 22 | 0.2 | | 0.10 | 0.21 | | 0.11 | 0.19 | | 0.09 |
| | | | | 23 | 0.48 | | 0.38 | 0.47 | | 0.37 | 0.36 | | 0.26 |
| | | | | 24 | 0.29 | | 0.19 | 0.29 | | 0.19 | 0.25 | | 0.15 |
| | | 3b | 5 | 25 | 0.79 | | 0.69 | 0.75 | | 0.85 | 0.53 | | 0.43 |
| | | | | 26 | 0.29 | | 0.19 | 0.3 | | 0.20 | 0.26 | | 0.16 |
| | | | | 27 | 0.33 | | 0.23 | 0.3 | | 0.20 | 0.31 | | 0.21 |
| | | | | 28 | 0.23 | | 0.13 | 0.26 | | 0.16 | 0.27 | | 0.17 |
| | | Average | | | | | 0.28 | | | 0.28 | | | 0.21 |
| | | SEM | | | | | 0.072 | | | 0.063 | | | 0.036 |
| | | STDEV | | | | | 0.20 | | | 0.19 | | | 0.10 |
| 4 | SU-5416/25 mgkd | 4a | 6 | 29 | 0.23 | | 0.13 | 0.2 | | 0.10 | 0.18 | | 0.08 |
| | Calecoxib/150 ppm | | | 30 | 0.4 | | 0.30 | 0.35 | | 0.25 | 0.33 | | 0.23 |
| | | | | 31 | 0.32 | | 0.22 | 0.3 | | 0.20 | 0.28 | | 0.16 |
| | | | | 32 | 0.19 | | 0.00 | 0.19 | | 0.09 | 0.19 | | 0.09 |
| | | 4b | 7 | 33 | 0.45 | | 0.35 | 0.5 | | 0.40 | 0.46 | | 0.36 |
| | | | | 34 | 0.38 | | 0.28 | 0.4 | | 0.30 | 0.35 | | 0.25 |
| | | | | 35 | 0.25 | | 0.15 | 0.23 | | 0.13 | 0.28 | | 0.18 |
| | | | | 36 | 0.23 | | 0.13 | 0.24 | | 0.14 | 0.26 | | 0.16 |
| | | Average | | | | | 0.21 | | | 0.20 | | | 0.18 |
| | | SEM | | | | | 0.034 | | | 0.030 | | | 0.032 |
| | | STDEV | | | | | 0.10 | | | 0.11 | | | 0.09 |
| 5 | SU-5416/50 mgkd | 5a | 9 | 37 | 0.18 | | 0.08 | 0.24 | | 0.14 | 0.25 | | 0.15 |
| | | | | 38 | 0.53 | | 0.43 | 0.6 | | 0.50 | 0.46 | | 0.36 |
| | | | | 39 | 0.15 | | 0.05 | 0.18 | | 0.08 | 0.19 | | 0.09 |
| | | | | 40 | 0.25 | | 0.15 | 0.21 | | 0.11 | 0.28 | | 0.18 |
| | | 5b | 14 | 41 | 0.69 | | 0.59 | 0.63 | | 0.73 | 0.52 | | 0.42 |
| | | | | 42 | 0.16 | | 0.08 | 0.24 | | 0.14 | 0.16 | | 0.08 |
| | | | | 43 | 0.85 | | 0.75 | 0.72 | | 0.62 | 0.74 | | 0.64 |
| | | | | 44 | 0.74 | | 0.64 | 0.68 | | 0.58 | 0.56 | | 0.46 |
| | | Average | | | | | 0.34 | | | 0.36 | | | 0.30 |
| | | SEM | | | | | 0.103 | | | 0.086 | | | 0.072 |
| | | STDEV | | | | | 0.29 | | | 0.27 | | | 0.20 |
| 6 | SU-5416/50 mgkd | 6a | 16 | 45 | 0.15 | | 0.05 | 0.19 | | 0.09 | 0.21 | | 0.11 |
| | Celecoxib/40 ppm | | | 46 | 0.52 | | 0.42 | 0.54 | | 0.44 | 0.39 | | 0.29 |
| | | | | 47 | 0.29 | | 0.19 | 0.38 | | 0.26 | 0.33 | | 0.23 |
| | | | | 48 | 0.41 | | 0.31 | 0.48 | | 0.38 | 0.32 | | 0.22 |
| | | 6b | 17 | 49 | 0.23 | | 0.13 | 0.25 | | 0.18 | 0.27 | | 0.17 |
| | | | | 50 | 0.46 | | 0.36 | 0.48 | | 0.38 | 0.44 | | 0.34 |
| | | | | 51 | 0.41 | | 0.31 | 0.35 | | 0.25 | 0.29 | | 0.19 |
| | | | | 52 | 0.31 | | 0.21 | 0.47 | | 0.37 | 0.41 | | 0.31 |
| | | Average | | | | | 0.26 | | | 0.29 | | | 0.23 |
| | | SEM | | | | | 0.044 | | | 0.043 | | | 0.027 |
| | | STDEV | | | | | 0.12 | | | 0.12 | | | 0.08 |
| 7 | SU-5416/50 mgkd | 7a | 18 | 53 | 0.22 | | 0.12 | 0.24 | | 0.14 | 0.21 | | 0.11 |
| | Calecoxib/60 ppm | | | 54 | 0.33 | | 0.23 | 0.33 | | 0.23 | 0.29 | | 0.19 |
| | | | | 55 | 0.5 | | 0.40 | 0.51 | | 0.41 | 0.48 | | 0.38 |
| | | | | 56 | 0.14 | | 0.04 | 0.19 | | 0.09 | 0.21 | | 0.11 |
| | | 7b | [symbol] | 57 | 0.18 | | 0.08 | 0.24 | | 0.14 | 0.23 | | 0.13 |
| | | | | 58 | 0.22 | | 0.12 | 0.21 | | 0.11 | 0.22 | | 0.12 |
| | | | | 59 | 0.24 | | 0.14 | 0.24 | | 0.14 | 0.24 | | 0.14 |
| | | | | 60 | | | | | | | | | |
| | | | | | | | 0.16 | | | 0.18 | | | 0.17 |
| | | | | | | | 0.048 | | | 0.042 | | | 0.037 |
| | | | | | | | 0.12 | | | 0.11 | | | 0.10 |
| 8 | Celecoxib/40 ppm | 8a | 24 | 61 | 0.7 | | 0.60 | 0.63 | | 0.53 | 0.37 | | 0.27 |
| | | | | 62 | 0.48 | | 0.38 | 0.51 | | 0.41 | 0.32 | | 0.22 |
| | | | | 63 | 0.25 | | 0.18 | 0.27 | | 0.17 | 0.23 | | 0.13 |
| | | | | 64 | 0.75 | | 0.55 | 0.72 | | 0.82 | 0.44 | | 0.34 |
| | | 8b | 26 | 65 | 0.44 | | 0.34 | 0.30 | | 0.29 | 0.28 | | 0.18 |
| | | | | 66 | 0.38 | | 0.28 | 0.39 | | 0.20 | 0.29 | | 0.19 |
| | | | | 67 | 0.89 | | 0.79 | 0.71 | | 0.61 | 0.47 | | 0.37 |
| | | | | 68 | 0.48 | | 0.38 | 0.41 | | 0.31 | 0.27 | | 0.17 |
| | | Average | | | | | 0.48 | | | 0.40 | | | 0.23 |
| | | SEM | | | | | 0.074 | | | 0.069 | | | 0.030 |
| | | STDEV | | | | | 0.21 | | | 0.17 | | | 0.09 |
| 9 | Celecoxib/160 ppm | 9a | 27 | 69 | 0.33 | | 0.23 | 0.38 | | 0.28 | 0.22 | | 0.12 |
| | | | | 70 | 0.33 | | 0.23 | 0.32 | | 0.22 | 0.28 | 0.1 | 0.16 |
| | | | | 71 | 0.33 | | 0.22 | 0.3 | | 0.20 | 0.24 | | 0.14 |
| | | | | 72 | 0.57 | | 0.47 | 0.49 | | 0.39 | 0.38 | | 0.28 |
| | | 9b | 28 | 73 | 0.29 | | 0.19 | 0.33 | 0.1 | 0.23 | 0.27 | | 0.17 |
| | | | | 74 | 0.59 | | 0.49 | 0.57 | | 0.47 | 0.35 | | 0.26 |
| | | | | 75 | 0.4 | | 0.30 | 0.4 | | 0.30 | 0.31 | | 0.21 |
| | | | | 76 | 0.28 | | 0.10 | 0.5 | | 0.20 | 0.24 | | 0.14 |
| | | | | | | | 0.29 | | | 0.29 | | | 0.19 |
| | | | | | | | 0.044 | | | 0.035 | | | 0.020 |
| | | | | | | | 0.12 | | | 0.10 | | | 0.06 |

| Mice Weighed day of injection | | | | | | | | | | | | |
|---|---|---|---|---|---|---|---|---|---|---|---|---|
| Paw volume | | | | | | | | | | | | |
| | | | | | Day 21 | | | Day 17 | | | Day 14 | |
| As- | | | | | Mar. 20, | | | Mar. 16, | | | Mar. 13, | |
| signed | | | original | | 2001 | BKG. | Tumor | 2001 | BKG. | Tumor | 2001 | BKG. |
| Group | | Cage # | cage | | paw | 0.10 | vol. | paw | 0.10 | vol. | paw | 0.10 |
| 1 | Vehicle | 1a | 10 | 1 | 0.20 | | 0.16 | 0.24 | | 0.14 | 0.24 | |
| | | | | 2 | 0.29 | 0.1 | 0.19 | 0.27 | | 0.17 | 0.28 | |
| | | | | 3 | 0.22 | | 0.12 | 0.23 | | 0.12 | 0.23 | |
| | | | | 4 | 0.26 | | 0.10 | 0.26 | | 0.16 | 0.24 | |
| | | 1b | 11 | 5 | 0.4 | | 0.30 | 0.3 | | 0.20 | 0.3 | |
| | | | | 6 | 0.52 | | 0.42 | 0.38 | | 0.28 | 0.32 | |
| | | | | 7 | 0.28 | | 0.18 | 0.24 | | 0.14 | 0.26 | |
| | | | | 8 | 0.46 | | 0.36 | 0.3 | | 0.20 | 0.29 | |
| | | | 20 | 9 | 0.36 | | 0.26 | 0.28 | | 0.18 | 0.23 | |
| | | | | 10 | 0.48 | | 0.38 | 0.32 | | 0.22 | 0.22 | |
| | | | | 11 | 0.27 | | 0.17 | 0.2 | | 0.10 | 0.23 | |
| | | | | 12 | 0.24 | | 0.14 | 0.2 | | 0.10 | 0.31 | |
| | | Average | | | | | 0.24 | | | 0.17 | | |
| | | BEM | | | | | 0.030 | | | 0.016 | | |
| | | STDEV | | | | | 0.10 | | | 0.05 | | |
| 2 | SU5416 | 2a | 2 | 13 | 0.55 | | 0.45 | 0.37 | | 0.27 | 0.29 | |
| | 25 mg/kg/day s.c. | | | 14 | 0.49 | | 0.39 | 0.33 | | 0.23 | 0.28 | |
| | | | | 15 | 0.37 | | 0.27 | 0.29 | | 0.19 | 0.27 | |
| | | | | 16 | 0.34 | | 0.24 | 0.28 | | 0.18 | 0.28 | |
| | | 2b | 3 | 17 | 0.4 | | 0.30 | 0.28 | | 0.18 | 0.26 | |
| | | | | 18 | 0.58 | | 0.48 | 0.36 | | 0.26 | 0.29 | 0.1 |
| | | | | 19 | 0.56 | | 0.46 | 0.37 | | 0.27 | 0.29 | |
| | | | | 20 | 0.24 | | 0.14 | 0.23 | | 0.13 | 0.24 | |
| | | Average | | | | | 0.34 | | | 0.21 | | |
| | | SEM | | | | | 0.043 | | | 0.010 | | |
| | | STDEV | | | | | 0.12 | | | 0.03 | | |
| 3 | SU-5416/25 mgkd | 3a | 4 | 21 | 0.3 | | 0.20 | 0.23 | | 0.13 | 0.26 | |
| | Calecoxib/40 ppm | | | 22 | 0.21 | | 0.11 | 0.25 | | 0.15 | 0.22 | |
| | | | | 23 | 0.33 | | 0.23 | 0.26 | | 0.18 | 0.29 | |
| | | | | 24 | 0.26 | | 0.16 | 0.27 | | 0.17 | 0.24 | |
| | | 3b | 5 | 25 | 0.43 | | 0.33 | 0.32 | | 0.22 | 0.31 | |
| | | | | 26 | 0.26 | | 0.16 | 0.25 | | 0.15 | 0.24 | |
| | | | | 27 | 0.29 | | 0.19 | 0.26 | | 0.18 | 0.26 | |
| | | | | 28 | 0.28 | | 0.14 | 0.27 | | 0.17 | 0.27 | |
| | | Average | | | | | 0.20 | | | 0.17 | | |
| | | SEM | | | | | 0.023 | | | 0.010 | | |
| | | STDEV | | | | | 0.06 | | | 0.03 | | |
| 4 | SU-5416/25 mgkd | 4a | 6 | 29 | 0.22 | | 0.12 | 0.22 | | 0.12 | 0.23 | |
| | Calecoxib/150 ppm | | | 30 | 0.31 | | 0.21 | 0.28 | | 0.18 | 0.26 | |
| | | | | 31 | 0.27 | | 0.17 | 0.26 | | 0.16 | 0.26 | |
| | | | | 32 | 0.21 | | 0.11 | 0.2 | | 0.10 | 0.22 | |
| | | 4b | 7 | 33 | 0.42 | | 0.32 | 0.37 | | 0.27 | 0.28 | |
| | | | | 34 | 0.38 | | 0.28 | 0.27 | | 0.17 | 0.27 | |
| | | | | 35 | 0.3 | | 0.20 | 0.28 | | 0.18 | 0.25 | |
| | | | | 36 | 0.23 | | 0.13 | 0.22 | | 0.12 | 0.24 | |
| | | Average | | | | | 0.19 | | | 0.17 | | |
| | | SEM | | | | | 0.027 | | | 0.019 | | |
| | | STDEV | | | | | 0.08 | | | 0.05 | | |
| 5 | SU-5416/50 mgkd | 5a | 9 | 37 | 0.27 | | 0.17 | 0.24 | | 0.14 | 0.29 | |
| | | | | 38 | 0.36 | | 0.28 | 0.27 | | 0.17 | 0.22 | |
| | | | | 39 | 0.19 | | 0.09 | 0.2 | | 0.10 | 0.32 | |
| | | | | 40 | 0.31 | | 0.21 | 0.3 | 0.1 | 0.20 | 0.27 | |
| | | 5b | 14 | 41 | 0.44 | | 0.34 | 0.32 | | 0.22 | 0.24 | |
| | | | | 42 | 0.22 | | 0.12 | 0.22 | | 0.12 | 0.26 | |
| | | | | 43 | 0.63 | | 0.53 | 0.39 | | 0.29 | 0.21 | |
| | | | | 44 | 0.48 | | 0.38 | 0.34 | | 0.24 | 0.27 | |
| | | Average | | | | | 0.27 | | | 0.19 | | |
| | | SEM | | | | | 0.062 | | | 0.023 | | |
| | | STDEV | | | | | 0.15 | | | 0.06 | | |
| 6 | SU-5416/50 mgkd | 6a | 16 | 45 | 0.21 | | 0.11 | 0.24 | | 0.14 | 0.23 | |
| | Celecoxib/40 ppm | | | 46 | 0.41 | | 0.31 | 0.31 | | 0.21 | 0.26 | |
| | | | | 47 | 0.37 | | 0.27 | 0.24 | | 0.18 | 0.26 | |
| | | | | 48 | 0.33 | | 0.23 | 0.26 | | 0.18 | 0.24 | |
| | | 6b | 17 | 49 | 0.27 | | 0.17 | 0.24 | | 0.14 | 0.21 | |
| | | | | 50 | 0.41 | | 0.31 | 0.31 | | 0.21 | 0.3 | |
| | | | | 51 | 0.30 | | 0.28 | 0.3 | | 0.20 | 0.28 | |
| | | | | 52 | 0.33 | | 0.23 | 0.3 | | 0.20 | 0.28 | |
| | | Average | | | | | 0.24 | | | 0.18 | | |
| | | SEM | | | | | [symbol] | | | 0.010 | | |
| | | STDEV | | | | | [symbol] | | | 0.03 | | |
| 7 | SU-5416/50 mgkd | 7a | 18 | 53 | 0.23 | | 0.13 | 0.25 | | 0.15 | 0.28 | |
| | Calecoxib/60 ppm | | | 54 | 0.27 | | 0.17 | 0.26 | | 0.18 | 0.25 | |
| | | | | 55 | 0.48 | | 0.38 | 0.44 | | 0.54 | 0.38 | |
| | | | | 56 | 0.2 | | 0.10 | 0.23 | | 0.13 | 0.2 | |
| | | 7b | [symbol] | 57 | 0.21 | | 0.11 | 0.21 | | 0.11 | 0.24 | |
| | | | | 58 | 0.25 | | 0.15 | 0.25 | | 0.15 | 0.22 | |
| | | | | 59 | 0.27 | | 0.17 | 0.25 | | 0.15 | 0.25 | |
| | | | | 60 | | | 0.25 | | 0.15 | 0.25 | | |
| | | | | | | | 0.17 | | | 0.17 | | |
| | | | | | | | 0.038 | | | 0.028 | | |
| | | | | | | | 0.10 | | | 0.07 | | |
| 8 | Celecoxib/40 ppm | 8a | 24 | 61 | 0.38 | | 0.28 | 0.3 | | 0.20 | 0.28 | |
| | | | | 62 | 0.38 | | 0.28 | 0.29 | | 0.19 | 0.25 | |
| | | | | 63 | 0.23 | | 0.13 | 0.22 | | 0.12 | 0.24 | |
| | | | | 64 | 0.36 | | 0.28 | 0.32 | | 0.22 | 0.29 | |
| | | 8b | 26 | 65 | 0.28 | | 0.18 | 0.27 | | 0.17 | 0.28 | |
| | | | | 66 | 0.28 | | 0.18 | 0.25 | | 0.15 | 0.25 | |
| | | | | 67 | 0.39 | | 0.29 | 0.29 | | 0.19 | 0.27 | |
| | | | | 68 | 0.31 | | 0.21 | 0.25 | | 0.15 | 0.24 | |
| | | Average | | | | | 0.22 | | | 0.17 | | |
| | | SEM | | | | | 0.021 | | | 0.011 | | |
| | | STDEV | | | | | 0.06 | | | 0.03 | | |
| 9 | Celecoxib/160 ppm | 9a | 27 | 69 | 0.22 | | 0.12 | 0.24 | | 0.14 | 0.19 | |
| | | | | 70 | 0.28 | | 0.16 | 0.25 | | 0.15 | 0.26 | |
| | | | | 71 | 0.24 | | 0.14 | 0.21 | | 0.11 | 0.22 | |
| | | | | 72 | 0.32 | | 0.22 | 0.31 | | 0.21 | 0.3 | |
| | | 9b | 28 | 73 | 0.28 | | 0.16 | 0.23 | | 0.13 | 0.23 | |
| | | | | 74 | 0.3 | | 0.20 | 0.29 | | 0.19 | 0.28 | |
| | | | | 75 | 0.3 | | 0.20 | 0.27 | | 0.17 | 0.23 | |
| | | | | 76 | 0.25 | | 0.16 | 0.25 | | 0.15 | 0.23 | |
| | | | | | | | 0.15 | | | 0.18 | | |
| | | | | | | | 0.012 | | | 0.011 | | |
| | | | | | | | 0.03 | | | 0.03 | | |

| Mice Weighed day of injection | | | | | | | | | | | |
|---|---|---|---|---|---|---|---|---|---|---|---|
| Paw volume | | | | | | | | | | | |
| | | | | | | Start | | | | | |
| | | | | | | dosing | | | | | |
| | | | | | | Day 10 | | | Day 7 | | |
| As- | | | | | | Mar. 9, | | | Mar. 3, | | |
| signed | | | original | | Tumor | 2001 | BKG. | Tumor | 2001 | | Tumor |
| Group | | Cage # | cage | | vol. | paw | 0.10 | vol. | paw | 0.10 | vol. |
| 1 | Vehicle | 1a | 10 | 1 | 0.14 | 0.26 | | 0.16 | 0.27 | | 0.17 |
| | | | | 2 | 0.10 | 0.28 | | 0.18 | 0.27 | | 0.17 |
| | | | | 3 | 0.13 | 0.27 | | 0.17 | 0.27 | | 0.17 |
| | | | | 4 | 0.14 | 0.25 | | 0.15 | 0.27 | | 0.17 |
| | | 1b | 11 | 5 | 0.20 | 0.20 | | 0.19 | 0.26 | | 0.15 |
| | | | | 6 | 0.22 | 0.3 | | 0.20 | 0.20 | | 0.10 |
| | | | | 7 | 0.16 | 0.25 | | 0.15 | 0.24 | | 0.14 |
| | | | | 8 | 0.19 | 0.26 | | 0.16 | 0.28 | | 0.16 |
| | | | 20 | 9 | 0.13 | 0.22 | | 0.12 | 0.21 | | 0.11 |
| | | | | 10 | 0.12 | 0.24 | | 0.18 | 0.27 | | 0.17 |
| | | | | 11 | 0.13 | 0.23 | | 0.13 | 0.2 | | 0.10 |
| | | | | 12 | 0.21 | 0.21 | | 0.11 | 0.2 | | 0.10 |
| | | Average | | | 0.14 | | | 0.16 | | | 0.16 |
| | | BEM | | | 0.010 | | | 0.008 | | | 0.008 |
| | | STDEV | | | 0.04 | | | 0.03 | | | 0.03 |
| 2 | SU5416 | 2a | 2 | 13 | 0.19 | 0.3 | | 0.20 | 0.26 | | 0.16 |
| | 25 mg/kg/day s.c. | | | 14 | 0.18 | 0.25 | | 0.15 | 0.28 | | 0.16 |
| | | | | 15 | 0.17 | 0.23 | | 0.13 | 0.27 | | 0.17 |
| | | | | 16 | 0.16 | 0.26 | 0.1 | 0.10 | 0.26 | | 0.17 |
| | | 2b | 3 | 17 | 0.16 | 0.27 | | 0.17 | 0.27 | | 0.17 |
| | | | | 18 | 0.19 | 0.29 | | 0.19 | 0.26 | | 0.18 |
| | | | | 19 | 0.19 | 0.24 | | 0.14 | 0.24 | | 0.14 |
| | | | | 20 | 0.14 | 0.24 | | 0.14 | 0.23 | | 0.13 |
| | | Average | | | 0.18 | | | 0.16 | | | 0.14 |
| | | SEM | | | 0.004 | | | 0.009 | | | 0.008 |
| | | STDEV | | | 0.02 | | | 0.03 | | | 0.02 |
| 3 | SU-5416/25 mgkd | 3a | 4 | 21 | 0.15 | 0.25 | | 0.15 | 0.24 | | 0.14 |
| | Calecoxib/40 ppm | | | 22 | 0.12 | 0.24 | | 0.14 | 0.25 | | 0.15 |
| | | | | 23 | 0.19 | 0.29 | | 0.19 | 0.26 | | 0.16 |
| | | | | 24 | 0.14 | 0.27 | | 0.17 | 0.25 | | 0.15 |
| | | 3b | 5 | 25 | 0.21 | 0.27 | | 0.17 | 0.29 | | 0.19 |
| | | | | 26 | 0.14 | 0.24 | | 0.14 | 0.22 | | 0.12 |
| | | | | 27 | 0.16 | 0.28 | | 0.10 | 0.20 | | 0.18 |
| | | | | 28 | 0.17 | 0.28 | | 0.18 | 0.25 | | 0.15 |
| | | Average | | | 0.16 | | | 0.17 | | | 0.14 |
| | | SEM | | | 0.010 | | | 0.007 | | | 0.009 |
| | | STDEV | | | 0.03 | | | 0.02 | | | 0.02 |
| 4 | SU-5416/25 mgkd | 4a | 6 | 29 | 0.13 | 0.27 | | 0.17 | 0.31 | | 0.21 |
| | Calecoxib/150 ppm | | | 30 | 0.16 | 0.29 | | 0.19 | 0.17 | | 0.17 |
| | | | | 31 | 0.16 | 0.27 | | 0.17 | 0.25 | | 0.15 |
| | | | | 32 | 0.12 | 0.25 | | 0.15 | 0.26 | | 0.16 |
| | | 4b | 7 | 33 | 0.18 | 0.31 | | 0.21 | 0.29 | | 0.19 |
| | | | | 34 | 0.17 | 0.28 | | 0.18 | 0.28 | | 0.18 |
| | | | | 35 | 0.15 | 0.28 | | 0.18 | 0.29 | | 0.10 |
| | | | | 36 | 0.14 | 0.26 | | 0.15 | 0.28 | | 0.18 |
| | | Average | | | 0.18 | | | 0.18 | | | 0.18 |
| | | SEM | | | 0.007 | | | 0.007 | | | 0.007 |
| | | STDEV | | | 0.02 | | | 0.02 | | | 0.02 |
| 5 | SU-5416/50 mgkd | 5a | 9 | 37 | 0.19 | 0.28 | | 0.16 | 0.24 | | 0.14 |
| | | | | 38 | 0.12 | 0.24 | | 0.14 | 0.26 | | 0.16 |
| | | | | 39 | 0.22 | 0.24 | | 0.14 | 0.22 | | 0.12 |
| | | | | 40 | 0.17 | 0.27 | | 0.17 | 0.25 | | 0.15 |
| | | 5b | 14 | 41 | 0.14 | 0.31 | | 0.21 | 0.27 | | 0.17 |
| | | | | 42 | 0.18 | 0.24 | | 0.14 | 0.23 | | 0.13 |
| | | | | 43 | 0.11 | 0.27 | | 0.17 | 0.24 | | 0.14 |
| | | | | 44 | 0.17 | 0.24 | | 0.14 | 0.24 | | 0.14 |
| | | Average | | | 0.18 | | | 0.16 | | | 0.14 |
| | | SEM | | | 0.013 | | | 0.009 | | | 0.006 |
| | | STDEV | | | 0.04 | | | 0.03 | | | 0.02 |
| 6 | SU-5416/50 mgkd | 6a | 16 | 45 | 0.13 | 0.24 | | 0.14 | 0.22 | | 0.12 |
| | Celecoxib/40 ppm | | | 46 | 0.16 | 0.32 | | 0.22 | 0.32 | | 0.22 |
| | | | | 47 | 0.16 | 0.27 | | 0.17 | 0.28 | | 0.18 |
| | | | | 48 | 0.14 | 0.28 | | 0.18 | 0.24 | | 0.14 |
| | | 6b | 17 | 49 | 0.11 | 0.28 | | 0.18 | 0.23 | | 0.13 |
| | | | | 50 | 0.20 | 0.31 | | 0.21 | 0.24 | | 0.14 |
| | | | | 51 | 0.14 | 0.28 | | 0.18 | 0.24 | | 0.14 |
| | | | | 52 | 0.16 | 0.27 | | 0.17 | 0.22 | | 0.12 |
| | | Average | | | 0.16 | | | 0.19 | | | 0.15 |
| | | SEM | | | 0.009 | | | 0.009 | | | 0.011 |
| | | STDEV | | | 0.03 | | | 0.03 | | | 0.03 |
| 7 | SU-5416/50 mgkd | 7a | 18 | 53 | 0.16 | 0.28 | | 0.18 | 0.25 | | 0.15 |
| | Calecoxib/60 ppm | | | 54 | 0.15 | 0.25 | | 0.15 | 0.22 | | 0.12 |
| | | | | 55 | 0.28 | 0.31 | | 0.21 | 0.28 | | 0.18 |
| | | | | 56 | 0.10 | 0.24 | | 0.14 | 0.2 | | 0.10 |
| | | 7b | [symbol] | 57 | 0.14 | 0.24 | | 0.14 | 0.23 | | 0.13 |
| | | | | 58 | 0.12 | 0.27 | | 0.17 | 0.24 | | 0.14 |
| | | | | 59 | 0.15 | 0.3 | | 0.20 | 0.25 | | 0.15 |
| | | | | 60 | 0.15 | 0.28 | | 0.15 | 0.25 | | 0.15 |
| | | | | | 0.18 | | | 0.17 | | | 0.14 |
| | | | | | 0.019 | | | 0.009 | | | 0.008 |
| | | | | | 0.05 | | | 0.03 | | | 0.02 |
| 8 | Celecoxib/40 ppm | 8a | 24 | 61 | 0.18 | 0.26 | | 0.15 | 0.25 | | 0.15 |
| | | | | 62 | 0.15 | 0.28 | | 0.18 | 0.27 | | 0.17 |
| | | | | 63 | 0.14 | 0.26 | | 0.18 | 0.24 | | 0.14 |
| | | | | 64 | 0.19 | 0.27 | | 0.17 | 0.25 | | 0.15 |
| | | 8b | 26 | 65 | 0.14 | 0.28 | | 0.18 | 0.27 | | 0.17 |
| | | | | 66 | 0.15 | 0.20 | | 0.18 | 0.25 | | 0.16 |
| | | | | 67 | 0.17 | 0.3 | | 0.20 | 0.29 | | 0.19 |
| | | | | 68 | 0.14 | 0.27 | | 0.17 | 0.25 | | 0.15 |
| | | Average | | | 0.18 | | | 0.17 | | | 0.10 |
| | | SEM | | | 0.007 | | | 0.006 | | | 0.006 |
| | | STDEV | | | 0.02 | | | 0.01 | | | 0.02 |
| 9 | Celecoxib/160 ppm | 9a | 27 | 69 | 0.09 | 0.23 | | 0.13 | 0.24 | | 0.14 |
| | | | | 70 | 0.18 | 0.27 | | 0.17 | 0.24 | 0.1 | 0.14 |
| | | | | 71 | 0.12 | 0.26 | | 0.16 | 0.23 | | 0.13 |
| | | | | 72 | 0.20 | 0.32 | | 0.22 | 0.3 | | 0.20 |
| | | 9b | 28 | 73 | 0.13 | 0.28 | | 0.18 | 0.20 | | 0.16 |
| | | | | 74 | 0.18 | 0.28 | 0.1 | 0.18 | 0.20 | | 0.19 |
| | | | | 75 | 0.13 | 0.27 | | 0.17 | 0.23 | | 0.13 |
| | | | | 76 | 0.13 | 0.23 | | 0.13 | 0.24 | | 0.14 |
| | | | | | 0.14 | | | 0.17 | | | 0.13 |
| | | | | | 0.013 | | | 0.010 | | | 0.010 |
| | | | | | 0.04 | | | 0.03 | | | 0.03 |

TABLE 7
Raw Data Showing Weight of Treated Mice

Mice Weighted day injection
Body weight

| | | | | | | | | | | Start dosing | | | |
|---|---|---|---|---|---|---|---|---|---|---|---|---|---|
| | | | | | Day 35 | Day 31 | Day 24 | Day 21 | Day 17 | Day 14 | Day 10 | Day 7 | Day 0 |
| As- | | | origi- | | Apr. 3, | Mar. 30, | Mar. 23, | Mar. 20, | Mar. 16, | Mar. 13, | Mar. 9, | Mar. 6, | Feb. 27, |
| signed | | | nal | | 2001 | 2001 | 2001 | 2001 | 2001 | 2001 | 2001 | 2001 | 2001 |
| Group | | Cage # | cage | | body wt | body wt | body wt | body wt | body wt | body wt | body wt | body wt | body wt |
| 1 | Vehicle | 1a | 10 | 1 | 34.05 | 34.53 | 32.01 | 31.21 | 30.27 | 33.41 | 31.08 | 30.69 | 29.67 |
| | | | | 2 | 35.79 | 35.65 | 34.34 | 33.58 | 33.17 | 30.59 | 32.53 | 33.15 | 31.8 |
| | | | | 3 | 31.61 | 32.69 | 30.51 | 30.16 | 28.77 | 28.33 | 29.2 | 29.75 | 28.71 |
| | | | | 4 | 28.41 | 29.12 | 27.45 | 27.65 | 26.78 | 25.91 | 26.31 | 26.39 | 25.27 |
| | | 1b | 11 | 1 | 24.4 | 26.69 | 28.03 | 28.62 | 28.26 | 27.97 | 28.71 | 28.62 | 26.9 |
| | | | | 2 | 28.67 | 30.43 | 31 | 32.04 | 32.14 | 32.09 | 32.31 | 32.04 | 30.01 |
| | | | | 3 | 27.68 | 27.86 | 27.6 | 27.3 | 26.49 | 26.02 | 26.34 | 26.6 | 24.99 |
| | | | | 4 | 31.24 | 32.35 | 31.72 | 32.29 | 31.57 | 29.87 | 30.41 | 30 | 28.46 |
| | | | 20 | 1 | 24.53 | 25.14 | 24.62 | 24.8 | 25.01 | 25.13 | 25.09 | 24.26 | 19.89 |
| | | | | 2 | 30.52 | 31.88 | 31.70 | 30.86 | 30.93 | 23.41 | 29.82 | 29.37 | 26.43 |
| | | | | 3 | 28.24 | 27.36 | 27.54 | 27.19 | 26.23 | 25.29 | 23.35 | 23.16 | 21.82 |
| | | | | 4 | 24.43 | 24.91 | 24.83 | 24.25 | 23.27 | 30.23 | 23.72 | 23.78 | 22 |
| | | Average | | | 29.13 | 29.88 | 29.28 | 29.16 | 28.57 | 28.19 | 28.24 | 28.15 | 26.33 |
| | | SEM | | | 1.067 | 1.036 | 0.879 | 0.865 | 0.893 | 0.895 | 0.925 | 0.949 | 1.061 |
| | | STDEV | | | 3.70 | 3.59 | 3.04 | 3.00 | 3.09 | 3.10 | 3.20 | 3.29 | 3.68 |
| 2 | SU5416 | 2a | 2 | 1 | 26.84 | 22.69 | 27.88 | 28.31 | 28.98 | 29.47 | 30.14 | 29.59 | 28.71 |
| | 25 mg/kg/day s.c. | | | 2 | 23.55 | 23.86 | 24.83 | 25.2 | 26.16 | 28.02 | 27.48 | 27.28 | 26.6 |
| | | | | 3 | 21.74 | 17.88 | 21.3 | 22.29 | 23.34 | 23.54 | 24.19 | 24.61 | 24.24 |
| | | | | 4 | 30.55 | 27.38 | 30.13 | 31.41 | 31.49 | 32.11 | 32.36 | 32.41 | 31.93 |
| | | 2b | 3 | 1 | 22.95 | 27.9 | 22.29 | 23.29 | 23.48 | 23.91 | 25.96 | 25.96 | 25.48 |
| | | | | 2 | 22.47 | 23.36 | 22.93 | 24.3 | 25.66 | 27.08 | 28.31 | 27.54 | 26.36 |
| | | | | 3 | 18.42 | 20.51 | 18.15 | 18.32 | 18.97 | 21.18 | 24.22 | 23.83 | 22.53 |
| | | | | 4 | 27.58 | 29.73 | 29.02 | 28.9 | 29.34 | 29.41 | 31 | 30.4 | 29.41 |
| | | Average | | | 24.26 | 24.16 | 24.57 | 25.25 | 25.93 | 26.84 | 27.96 | 27.70 | 26.91 |
| | | SEM | | | 1.357 | 1.408 | 1.473 | 1.477 | 1.421 | 1.297 | 1.085 | 1.042 | 1.064 |
| | | STDEV | | | 3.84 | 3.98 | 4.17 | 4.18 | 4.02 | 3.67 | 3.07 | 2.95 | 3.01 |
| 3 | SU-5416/25 mpkd | 3a | 4 | 1 | 22.7 | 21.56 | 22.21 | 23.97 | 24.56 | 25.66 | 26.05 | 26.54 | 25.11 |
| | Celecoxib/40 ppm | | | 2 | 26.34 | 25.73 | 26.39 | 27.35 | 27.5 | 29.34 | 29.99 | 29.46 | 28.34 |
| | | | | 3 | 27.14 | 27.5 | 28.8 | 29.09 | 29.18 | 29.05 | 30.72 | 29.54 | 28.7 |
| | | | | 4 | 30.07 | 28.95 | 30.5 | 30.45 | 30.85 | 29.98 | 31.17 | 29.9 | 28.09 |
| | | 3b | 5 | 1 | 24.24 | 25.18 | 25.53 | 25.92 | 27.95 | 27.88 | 28.84 | 28.04 | 26.8 |
| | | | | 2 | 24.5 | 25.17 | 25.93 | 26.08 | 27 | 27.06 | 27.43 | 27.19 | 26.03 |
| | | | | 3 | 27.05 | 26.66 | 26.96 | 26.11 | 27.77 | 28.66 | 30.07 | 29.89 | 29.66 |
| | | | | 4 | 26.83 | 26.82 | 28.13 | 28.78 | 29.68 | 29.96 | 30.75 | 29.92 | 29.82 |
| | | Average | | | 26.11 | 25.95 | 26.81 | 27.22 | 28.06 | 28.45 | 29.38 | 28.81 | 27.82 |
| | | SEM | | | 0.801 | 0.770 | 0.877 | 0.746 | 0.674 | 0.532 | 0.640 | 0.480 | 0.599 |
| | | STDEV | | | 2.27 | 2.18 | 2.48 | 2.11 | 1.91 | 1.51 | 1.81 | 1.36 | 1.69 |
| 4 | SU-5416/25 mpkd | 4a | 6 | 1 | 29.3 | 29.62 | 29.59 | 30.39 | 29.36 | 28.52 | 29.38 | 30.03 | 28.66 |
| | Celecoxib/160 ppm | | | 2 | 27.3 | 27.34 | 27.06 | 28.34 | 27.64 | 23.54 | 29.08 | 28.76 | 27.8 |
| | | | | 3 | 25.57 | 25.88 | 26.82 | 27.9 | 27.14 | 25.16 | 27.64 | 27.86 | 27.32 |
| | | | | 4 | 27.18 | 27.29 | 26.95 | 28.02 | 27.17 | 23.83 | 27.2 | 27.4 | 27.23 |
| | | 4b | 7 | 1 | 24.06 | 23.51 | 25.56 | 25.6 | 27.06 | 25.24 | 28.6 | 28.82 | 27.54 |
| | | | | 2 | 24.95 | 25.11 | 25.34 | 25.58 | 26.52 | 27.49 | 28.31 | 28.62 | 27.69 |
| | | | | 3 | 26.52 | 27.1 | 28.59 | 28.65 | 29.87 | 31.38 | 31.43 | 30.77 | 29.58 |
| | | | | 4 | 25.5 | 25.76 | 28.46 | 29.4 | 30.72 | 28.53 | 31.23 | 30.77 | 30.44 |
| | | Average | | | 26.30 | 26.45 | 27.30 | 27.99 | 28.19 | 26.71 | 29.11 | 29.13 | 28.28 |
| | | SEM | | | 0.580 | 0.644 | 0.527 | 0.595 | 0.553 | 0.963 | 0.545 | 0.449 | 0.415 |
| | | STDEV | | | 1.64 | 1.82 | 1.49 | 1.68 | 1.56 | 2.72 | 1.54 | 1.27 | 1.17 |
| 5 | SU-5416/50 mpkd | 5a | 9 | 1 | 25.06 | 23.48 | 24.36 | 26.09 | 26.78 | 27.6 | 29.2 | 29.04 | 26.74 |
| | | | | 2 | 25.86 | 25.06 | 25.64 | 26.64 | 26.26 | 27.47 | 27.93 | 27.85 | 25.91 |
| | | | | 3 | 22.4 | 22.49 | 22.85 | 23.59 | 24.3 | 25.89 | 27.67 | 27.13 | 26.04 |
| | | | | 4 | 23.59 | 22.54 | 24.01 | 25.96 | 26.57 | 27.54 | 28.95 | 28.82 | 26.14 |
| | | 5b | 14 | 1 | 23.51 | 24.94 | 24.93 | 25.64 | 26.39 | 27.7 | 29.52 | 29.12 | 27.7 |
| | | | | 2 | 27.79 | 26.64 | 26.23 | 26.74 | 27.92 | 29.06 | 30.71 | 29.67 | 27.87 |
| | | | | 3 | 26.37 | 26.03 | 24.79 | 24.96 | 26.1 | 27.27 | 28.54 | 28.34 | 28.26 |
| | | | | 4 | 25.74 | 26.47 | 25.24 | 25.2 | 25.89 | 26.48 | 27.84 | 27.09 | 22.8 |
| | | Average | | | 25.04 | 24.71 | 24.76 | 25.60 | 26.28 | 27.38 | 28.80 | 28.38 | 26.43 |
| | | SEM | | | 0.625 | 0.596 | 0.368 | 0.363 | 0.357 | 0.329 | 0.363 | 0.337 | 0.610 |
| | | STDEV | | | 1.77 | 1.69 | 1.04 | 1.03 | 1.01 | 0.93 | 1.03 | 0.95 | 1.72 |
| 6 | SU-5416/50 mpkd | 6a | 16 | 1 | 24.4 | 24.54 | 25.93 | 27.13 | 28.04 | 29.3 | 28.07 | 27.22 | 26.32 |
| | Celecoxib/40 ppm | | | 2 | 26.47 | 27.19 | 28.22 | 28.37 | 28.41 | 28.5 | 30.2 | 29.55 | 29.08 |
| | | | | 3 | 23.45 | 23.76 | 24.7 | 26.07 | 25.62 | 26.33 | 29.04 | 27.67 | 26.85 |
| | | | | 4 | 23.22 | 24.02 | 25.33 | 26.01 | 27.45 | 28.25 | 29.39 | 28.96 | 26.18 |
| | | 6b | 17 | 1 | 22.72 | 22.4 | 24.43 | 24.72 | 25.96 | 26.36 | 27.21 | 26.72 | 25.79 |
| | | | | 2 | 22.15 | 20.41 | 20.06 | 21.42 | 23.29 | 25.34 | 26.52 | 26.77 | 25.48 |
| | | | | 3 | 25.19 | 22.68 | 25.07 | 25.2 | 26.06 | 26.88 | 28.39 | 28.32 | 26.55 |
| | | | | 4 | 22.51 | 25.5 | 26.2 | 25.82 | 26.5 | 27.6 | 28.95 | 28.45 | 26.84 |
| | | Average | | | 23.76 | 23.81 | 24.99 | 25.59 | 26.42 | 27.32 | 28.47 | 27.96 | 26.64 |
| | | SEM | | | 0.525 | 0.728 | 0.819 | 0.718 | 0.574 | 0.468 | 0.422 | 0.366 | 0.388 |
| | | STDEV | | | 1.48 | 2.06 | 2.32 | 2.03 | 1.62 | 1.32 | 1.19 | 1.03 | 1.10 |
| 7 | SU-5416/50 mpkd | 7a | 18 | 1 | 26.56 | 27.24 | 27.67 | 28.07 | 28.59 | 29.78 | 29.5 | 29.45 | 28.09 |
| | Celecoxib/160 ppm | | | 2 | 24.28 | 24.29 | 23.73 | 23.73 | 24 | 30.63 | 25.25 | 24.72 | 23.59 |
| | | | | 3 | 23.29 | 23.07 | 23.11 | 23.29 | 24.59 | 31.22 | 26.99 | 26.54 | 25.81 |
| | | | | 4 | 19.36 | 20.41 | 20.92 | 21.89 | 22.82 | 28.91 | 25.01 | 24.09 | 22.64 |
| | | 7b | 22 | 1 | 24.95 | 24.2 | 24.01 | 23.25 | 24.46 | 29.64 | 26.08 | 25.07 | 24.19 |
| | | | | 2 | 27.8 | 27.46 | 27.31 | 26.37 | 27.54 | 30.21 | 28.56 | 27.46 | 27.69 |
| | | | | 3 | 31.53 | 31.15 | 31.69 | 30.98 | 30.68 | 34.55 | 31.26 | 30.33 | 29.56 |
| | | | | 4 | | | | | 28.09 | 27.27 | 29.82 | 29.2 | 28.64 |
| | | | | | 25.40 | 25.40 | 25.49 | 25.37 | 26.35 | 30.28 | 27.81 | 27.11 | 26.28 |
| | | | | | 1.440 | 1.325 | 1.366 | 1.228 | 0.972 | 0.742 | 0.817 | 0.841 | 0.913 |
| | | | | | 3.81 | 3.50 | 3.61 | 3.25 | 2.75 | 2.10 | 2.31 | 2.38 | 2.58 |
| 8 | Celecoxib/40 ppm | 8a | 24 | 1 | 29.46 | 30.26 | 30.04 | 29.74 | 29.59 | 29.2 | 29.2 | 28.79 | 28.67 |
| | | | | 2 | 32.46 | 32.88 | 31.99 | 31.46 | 30.83 | 28.42 | 30.66 | 29.73 | 29.13 |
| | | | | 3 | 31.41 | 31.78 | 31.3 | 31.11 | 31.09 | 27.85 | 30.96 | 30.34 | 29.59 |
| | | | | 4 | 29.65 | 30.11 | 29.94 | 29.53 | 29.36 | 27.08 | 28.27 | 27.88 | 28.06 |
| | | 8b | 26 | 1 | 30.23 | 30.1 | 30.11 | 29.09 | 29.59 | 27.49 | 27.82 | 27.8 | 27.38 |
| | | | | 2 | 31.21 | 31.55 | 31.12 | 30.62 | 30.49 | 26.65 | 30.84 | 33.05 | 29.07 |
| | | | | 3 | 34.39 | 34.5 | 34.23 | 34.37 | 34.48 | 30.45 | 32.99 | 29.6 | 31.1 |
| | | | | 4 | 26.43 | 27.24 | 28.01 | 27.43 | 27.19 | 30.63 | 27.04 | 26.16 | 25.01 |
| | | Average | | | 30.66 | 31.05 | 30.84 | 30.42 | 30.33 | 28.47 | 29.72 | 29.17 | 28.50 |
| | | SEM | | | 0.830 | 0.767 | 0.642 | 0.722 | 0.732 | 0.530 | 0.701 | 0.728 | 0.631 |
| | | STDEV | | | 2.35 | 2.17 | 1.82 | 2.04 | 2.07 | 1.50 | 1.98 | 2.06 | 1.79 |
| 9 | Celecoxib/160 ppm | 9a | 27 | 1 | 30.7 | 29.08 | 25.72 | 25.43 | 25.39 | 25.77 | 25.35 | 24.92 | 23.85 |
| | | | | 2 | 28.58 | 29.1 | 28.98 | 28.5 | 28.01 | 28 | 27.51 | 26.75 | 24.71 |
| | | | | 3 | 30.12 | 30.01 | 29.62 | 28.62 | 28.12 | 28.65 | 27.91 | 27.81 | 27.06 |
| | | | | 4 | 30.41 | 31.67 | 31.6 | 31.34 | 31.82 | 32.28 | 33.27 | 32.64 | 29.88 |
| | | 9b | 28 | 1 | 23.01 | 24.25 | 30.48 | 30.38 | 30.02 | 30.36 | 30.98 | 30.69 | 29.69 |
| | | | | 2 | 31.33 | 32.44 | 31.02 | 30.71 | 31.18 | 31.23 | 30.01 | 29.2 | 28.33 |
| | | | | 3 | 32.87 | 33.12 | 32.36 | 32.05 | 32.31 | 32.27 | 32.66 | 32.11 | 30.97 |
| | | | | 4 | 29.34 | 29.77 | 28.23 | 28.5 | 26.87 | 27.98 | 28.43 | 27.52 | 25.77 |
| | | | | | 29.55 | 29.93 | 29.75 | 29.44 | 29.22 | 29.57 | 29.52 | 28.96 | 27.53 |
| | | | | | 1.038 | 0.975 | 0.750 | 0.749 | 0.883 | 0.827 | 0.959 | 0.955 | 0.921 |
| | | | | | 2.94 | 2.76 | 2.12 | 2.12 | 2.50 | 2.34 | 2.71 | 2.70 | 2.60 |

1-74. (canceled)

75. A combination comprising a 3-heteroaryl-2-indolinone compound or a pharmaceutically acceptable salt or prodrug thereof and a cyclooxygenase-2 selective inhibitor or a pharmaceutically acceptable salt or prodrug thereof, in amounts effective when used in combination therapy for treatment or prevention of neoplasia or a neoplasia-related disorder.

76. The combination of claim 75, wherein the 3-heteroaryl-2-indolinone compound is a compound of formula 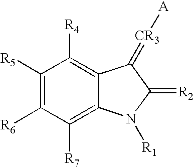
wherein:
R₁ is H or alkyl;
R₂ is O or S;
R₃ is hydrogen,
R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R and CONRR′;
A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole and tetrazole, optionally substituted at one or more positions with alkyl, alkoxy, aryl, aryloxy, alkaryl, akaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R and CONRR′;
n is 0-3;
R is H, alkyl or aryl; and
R′ is H, alkyl or aryl;
or a pharmaceutically acceptable salt or prodrug thereof.

77. The combination of claim 75, wherein the 3-heteroaryl-2-indolinone compound is 3-[(2,4-dimethylpyrrol-5-yl)methylene]-2-indolinone or a pharmaceutically acceptable salt or prodrug thereof.

78. The combination of claim 75, wherein the cyclooxygenase-2 selective inhibitor is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX-189, ABT-963, meloxicam, pharmaceutically acceptable salts of any of them, prodrugs of any of them, and mixtures thereof.

79. The combination of claim 75, wherein the cyclooxygenase-2 selective inhibitor is a phenylacetic acid derivative of formula 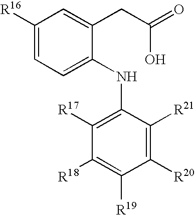
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl;
provided that R¹⁷, R¹⁸, R¹⁹ and R²⁰ are not all fluoro when R¹⁶ is ethyl and R¹⁹ is hydrogen; or a pharmaceutically acceptable salt or prodrug thereof.

80. The combination of claim 79, wherein, in the formula for the cyclooxygenase-2 selective inhibitor, R¹⁶ is ethyl; R¹⁷ and R¹⁹ are chloro; R¹⁸ and R²⁰ are hydrogen; and R²¹ is methyl.

81. A method for treating or preventing a neoplasia disorder in a subject, the method comprising administering in combination therapy to the subject a 3-heteroaryl-2-indolinone compound or a pharmaceutically acceptable salt or prodrug thereof and a cyclooxygenase-2 selective inhibitor or pharmaceutically acceptable salt or prodrug thereof, in amounts effective when used in said combination therapy for treatment or prevention of the neoplasia or neoplasia-related disorder.

82. The method of claim 81, wherein the 3-heteroaryl-2-indolinone compound is a compound of formula 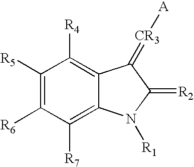
wherein:
R₁ is H or alkyl;
R₂ is O or S;
R₃ is hydrogen,
R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R and CONRR′;
A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole and tetrazole, optionally substituted at one or more positions with alkyl, alkoxy, aryl, aryloxy, alkaryl, akaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R and CONRR′;
n is 0-3;
R is H, alkyl or aryl; and
R′ is H, alkyl or aryl;
or a pharmaceutically acceptable salt or prodrug thereof.

83. The method of claim 81, wherein the 3-heteroaryl-2-indolinone compound is 3-[(2,4-dimethylpyrrol-5-yl)methylene]-2-indolinone or a pharmaceutically acceptable salt or prodrug thereof.

84. The method of claim 81, wherein the cyclooxygenase-2 selective inhibitor is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX-189, ABT-963, meloxicam, pharmaceutically acceptable salts of any of them, prodrugs of any of them, and mixtures thereof.

85. The method of claim 81, wherein the cyclooxygenase-2 selective inhibitor is a phenylacetic acid derivative of formula 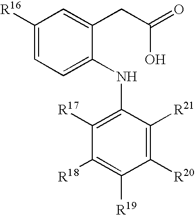
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl;
provided that R¹⁷, R¹⁸, R¹⁹ and R²⁰ are not all fluoro when R¹⁶ is ethyl and R¹⁹ is hydrogen; or a pharmaceutically acceptable salt or prodrug thereof.

86. The method of claim 81, wherein, in the formula for the cyclooxygenase-2 selective inhibitor, R¹⁶ is ethyl; R¹⁷ and R¹⁹ are chloro; R¹⁸ and R²⁰ are hydrogen; and R²¹ is methyl.

87. The method of claim 81, wherein the neoplasia is selected from the group consisting of acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary carcinoma, carcinoids, carcinoma, carcinosarcoma, cavernous neoplasia, cholangiocarcinoma, chondrosarcoma, chorioid plexus papilloma and carcinoma, clear cell carcinoma, cystadenoma, endodermal sinus tumor, endometrial hyperplasia, endometrial stromal sarcoma, endometrial adenocarcinoma, ependymal cancer, epithelioid carcinoma, Ewing's sarcoma, fibrolamellar carcinoma, focal nodular hyperplasia, gastrinoma, germ cell tumors, glioblastoma, glucagonoma, hemangioblastomas, hemangioendothelioma, hemangiomas, hepatic adenoma, hepatic adenomatosis, hepatocellular carcinoma, insulinoma, interepithelial squamous cell neoplasia, intraepithelial neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal carcinoma, mesothelial carcinoma, metastatic carcinoma, mucoepidermoid carcinoma, neuroblastoma, neuroepithelial adenocarcinoma, nodular melanoma, oat cell carcinoma, oligodendroglial carcinoma, osteosarcoma, papillary serous adenocarcinoma, pineal cell carcinoma, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous cell carcinoma, submesothelial carcinoma, superficial spreading melanoma, undifferentiated carcinoma, uveal melanoma, verrucous carcinoma, vipoma, well differentiated carcinoma, and Wilm's tumor.

88. The method of claim 81, wherein the neoplasia or neoplasia-related disorder is lung cancer.

89. The method of claim 81, wherein the neoplasia or neoplasia-related disorder is colorectal cancer.

90. The method of claim 81, wherein the neoplasia or neoplasia-related disorder is breast cancer.

91. The method of claim 81, wherein the neoplasia or neoplasia-related disorder is prostate cancer.

92. The method of claim 81, wherein the neoplasia or neoplasia-related disorder is bladder cancer.

93. The method of claim 81, wherein the neoplasia or neoplasia-related disorder is ovary cancer.

94. The method of claim 81, wherein the neoplasia or neoplasia-related disorder is cervical cancer.

95. The method of claim 81, wherein the neoplasia or neoplasia-related disorder is gastrointestinal cancer.

96. The method of claim 81, wherein the neoplasia or neoplasia-related disorder is head and neck cancer.

97. The method of claim 81, wherein the combination is administered in a sequential manner.

98. The method of claim 81, wherein the combination is administered in a substantially simultaneous manner.

99. The method of claim 81, wherein the 3-heteroaryl-2-indolinone compound or pharmaceutically acceptable salt or prodrug thereof is administered in an amount of about 0.01 to about 20 mg/day.

100. The method of claim 81, wherein the 3-heteroaryl-2-indolinone compound or pharmaceutically acceptable salt or prodrug thereof is administered orally.

101. The method of claim 81, wherein the 3-heteroaryl-2-indolinone compound or pharmaceutically acceptable salt or prodrug thereof is administered topically as a solution, cream, ointment, gel, lotion, suspension or emulsion.

102. The method of claim 101, wherein the 3-heteroaryl-2-indolinone compound or pharmaceutically acceptable salt or prodrug thereof is present in the solution, cream, ointment, gel, lotion, suspension or emulsion in an amount of about 0.01% to about 10%.

103. The method of claim 81, wherein the 3-heteroaryl-2-indolinone compound or pharmaceutically acceptable salt or prodrug thereof is administered intravenously.

104. A pharmaceutical composition comprising the combination of claim 1 and a pharmaceutically acceptable carrier.

105. A kit comprising a first dosage form that comprises a 3-heteroaryl-2-indolinone compound or a pharmaceutically acceptable salt or prodrug thereof in a first amount, and a second dosage form that comprises a cyclooxygenase-2 selective inhibitor or a pharmaceutically acceptable salt or prodrug thereof in a second amount; wherein said first and second amounts are effective when used in combination therapy for treating or preventing neoplasia or a neoplasia-related disorder.
